(19) United States
(12) Patent Application Publication
(10) Pub. No.: US 2026/0367516 A1
(43) Pub. Date: Nov. 19, 2026

(54) PHOTORESIST COMPOSITIONS AND METHODS OF FORMING PATTERNS

(71) Applicants: DuPont Electronic Materials International, LLC, Marlborough, MA (US); DuPont Electronics, Inc., Wilmington, DE (US)

(72) Inventors: Wenxu Zhang, Wellesley, MA (US); Le Zhou, Boylston, MA (US); Hua Dong, Shrewsbury, MA (US); Greg A. Hostetler, Newark, DE (US); Paul J. LaBeaume, Auburn, MA (US); Deyan Wang, Hudson, MA (US)

(21) Appl. No.: 19/660,757

(22) Filed: Apr. 28, 2026

Related U.S. Application Data
(60) Provisional application No. 63/797,703, filed on Apr. 30, 2025.

(51) Int. Cl. G03F 7/039; C07C 251/66; C08F 20/36; C08F 20/58; C08L 33/06; G03F 7/16; H10P 76/20

(52) U.S. Cl. CPC G03F 7/0397; C07C 251/66; C08F 20/36; C08F 20/58; C08L 33/062; G03F 7/162; G03F 7/168; H10P 76/2041

(57) ABSTRACT

A photoresist composition comprising a first polymer comprising a first repeating unit comprising a group of Formula (1) or (2); a second polymer comprising a first repeating unit comprising an acid-labile group; a photoacid generator; and a solvent,

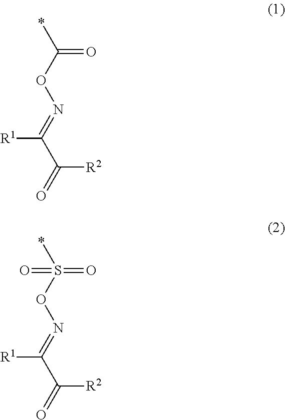

wherein in Formula (1) and Formula (2), R¹ and R² are each independently hydrogen or an organic group; R¹ and R² together optionally form a ring; each of R¹ and R² optionally further comprises one or more divalent linking groups as part of its structure; one of R¹ or R² is optionally bonded to a backbone of the first polymer; and * represents a point of bonding in the first repeating unit of the first polymer.

### CROSS-REFERENCE TO RELATED APPLICATIONS

This application claims priority to and the benefit of U.S. Provisional Application No. 63/797,703, filed on Apr. 30, 2025, the entire content of which is incorporated herein by reference.

### FIELD

The present invention relates to photoresist compositions and to pattern formation methods using such photoresist compositions. The photoresist compositions and pattern formation methods find particular use in lithographic applications in the semiconductor manufacturing industry.

### BACKGROUND

Photoresist compositions are photosensitive materials used to transfer a pattern to one or more underlying layers, such as a metal, semiconductor, or dielectric layer disposed on a substrate. Chemically amplified photoresist compositions are conventionally used for high-resolution processing. Such resist compositions typically include a polymer having acid-labile groups and a photoacid generator (PAG). A layer of the photoresist composition is pattern-wise exposed to activating radiation and the PAG generates an acid in the exposed regions. During post-exposure baking, the acid causes cleavage of the polymer's acid-labile groups and a resulting polarity-switch of the polymer in the exposed regions. This creates a difference in solubility characteristics between exposed and unexposed regions of the photoresist layer in a developer solution. In a positive tone development (PTD) process, exposed regions of the photoresist layer become soluble in a developer, typically an aqueous base developer, and are removed from the substrate surface while unexposed regions remain on the substrate to form a positive relief image. Alternatively, in a negative tone development (NTD) process, unexposed regions of the photoresist layer can be removed with an organic solvent developer, typically n-butyl acetate, while the exposed regions remain on the substrate to form a negative relief image. The resulting relief image permits selective processing of the substrate.

To improve lithographic performance, immersion lithography exposure tools have been developed to effectively increase the numerical aperture (NA) of the lens of the imaging device, for example, a scanner having an ArF light source. This is accomplished by use of a relatively high refractive index fluid (i.e., an immersion fluid), typically water, between the last surface of the imaging device and the surface of the photoresist layer on the semiconductor wafer. The immersion fluid allows for an increased amount of activating radiation from the light source to be directed into the photoresist layer.

Direct contact between the immersion fluid and photoresist layers formed from traditional non-immersion photoresist compositions would generally result in leaching of components of the photoresist into the immersion fluid. Such leaching can cause contamination of the scanner's optical lens and bring about a change in the effective refractive index and transmission properties of the immersion fluid. As one way to address this problem, photoresist compositions having an additive polymer that can self-segregate from other polymers in the composition to the photoresist layer upper surface during the spin-coating process have been developed and commonly used in the semiconductor manufacturing industry. A barrier layer is thereby formed in the photoresist layer which serves as the interface with the immersion fluid during immersion exposure. See, for example, U.S. Patent Application Pub. No. US20110255061 A1. The self-segregating property of the additive polymer is a result of its hydrophobic nature relative to other polymers in the photoresist composition. In the coated photoresist layer, the barrier layer, also as a result of the additive polymer's hydrophobicity, allows for a desirable (high) contact angle with the immersion fluid to minimize or prevent leaching of the photoresist components into the immersion fluid and prevent immersion fluid from entering the photoresist.

Certain fluorinated polymers useful as such additive polymers in immersion photoresists, while allowing for the formation of barrier layers during spin coating having desirable properties, are becoming of increased interest to the semiconductor manufacturing industry and governmental regulatory bodies for replacement with more sustainable alternatives. It would therefore be desirable to have a photoresist composition containing a barrier layer-forming additive polymer that does not rely on particular fluorine substitution.

There is a continuing need for photoresist compositions that address one or more problems associated with the state of the art, and for patterning methods using such photoresist compositions.

### SUMMARY

An aspect provides a photoresist composition that includes a first polymer comprising a first repeating unit comprising a group of Formula (1) or (2); a second polymer comprising a first repeating unit comprising an acid-labile group; a photoacid generator; and a solvent,

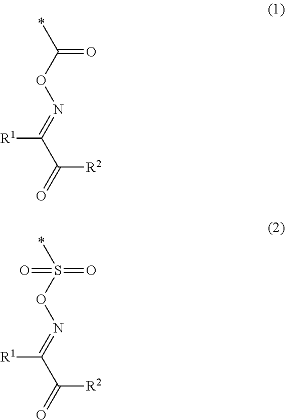

wherein in Formula (1) and Formula (2), R¹ and R² are each independently hydrogen or an organic group; R¹ and R² together optionally form a ring; each of R¹ and R² optionally further comprises one or more divalent linking groups as part of its structure; one of R¹ or R² is optionally bonded to a backbone of the first polymer; and * represents a point of bonding in the first repeating unit of the first polymer.

Another aspect provides a pattern formation method that includes (a) applying a layer of the photoresist composition of any one of claims 1 to 10 on a substrate; (b) pattern-wise exposing the photoresist composition layer to activating radiation; and (c) developing the exposed photoresist composition layer to provide a resist relief image.

Still another aspect provides a monomer including an ethylenically unsaturated double bond and a group of Formula (1A) or (2A):

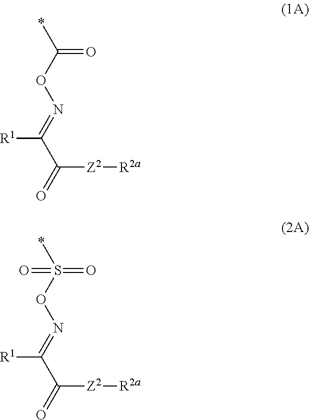

wherein, in Formulae (1A) and (2A), R¹ is hydrogen, substituted or unsubstituted C₁₋₃₀ alkyl, substituted or unsubstituted C₁₋₃₀ heteroalkyl, substituted or unsubstituted C₃₋₃₀ cycloalkyl, substituted or unsubstituted C₂₋₃₀ heterocycloalkyl, substituted or unsubstituted C₂₋₃₀ alkenyl, or substituted or unsubstituted C₂₋₃₀ heteroalkenyl; R^{2a} is an organic group; Z² is NR′, O, or S; R′ is hydrogen, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl; R^{1a} and R^{2a} together optionally form a ring; R^{1a} and R^{2a} each optionally further comprises one or more divalent linking groups as part of its structure; * represents a point of bonding in the monomer.

Another aspect provides a polymer comprising a repeating unit comprising a group of Formula (1A) or (2A):

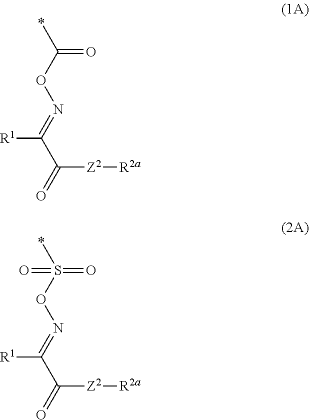

wherein, in Formulae (1A) and (2A), R¹ is hydrogen, substituted or unsubstituted C₁₋₃₀ alkyl, substituted or unsubstituted C₁₋₃₀ heteroalkyl, substituted or unsubstituted C₃₋₃₀ cycloalkyl, substituted or unsubstituted C₂₋₃₀ heterocycloalkyl, substituted or unsubstituted C₂₋₃₀ alkenyl, or substituted or unsubstituted C₂₋₃₀ heteroalkenyl; R^{2a} is an organic group; Z² is NR′, O, or S; R′ is hydrogen, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl; R¹ and R^{2a} together optionally form a ring; R¹ and R^{2a} each optionally further comprises one or more divalent linking groups as part of its structure; one of R¹ or R^{2a} is optionally bonded to a backbone of the polymer; and * represents a point of bonding in the repeating unit of the polymer.

### DETAILED DESCRIPTION

Reference will now be made in detail to exemplary embodiments, examples of which are illustrated in the present description. In this regard, the present exemplary embodiments may have different forms and should not be construed as being limited to the descriptions set forth herein. Accordingly, the exemplary embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

As used herein, the terms “a,” “an,” and “the” do not denote a limitation of quantity and are to be construed to cover both the singular and the plural, unless otherwise indicated herein or clearly contradicted by context. “Or” means “and/or” unless clearly indicated otherwise. The modifier “about” used in connection with a quantity is inclusive of the stated value and has the meaning dictated by the context (e.g., includes the degree of error associated with measurement of the particular quantity). All ranges disclosed herein are inclusive of the endpoints, and the endpoints are independently combinable with each other. The suffix “(s)” is intended to include both the singular and the plural of the term that it modifies, thereby including at least one of that term. “Optional” or “optionally” means that the subsequently described event or circumstance can or cannot occur, and that the description includes instances where the event occurs and instances where it does not. The terms “first,” “second,” and the like, herein do not denote an order, quantity, or importance, but rather are used to distinguish one element from another. When an element is referred to as being “on” another element, it may be directly in contact with the other element or intervening elements may be present therebetween. In contrast, when an element is referred to as being “directly on” another element, there are no intervening elements present. It is to be understood that the described components, elements, limitations, and/or features of aspects may be combined in any suitable manner in the various aspects.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used herein, “actinic rays” or “radiation” means, for example, a bright line spectrum of a mercury lamp, far ultraviolet rays represented by an excimer laser, extreme ultraviolet rays (EUV light), X-rays, particle rays such as electron beams and ion beams, or the like. In addition, in the present invention, “light” means actinic rays or radiation. The krypton fluoride laser (KrF laser) is a particular type of excimer laser, which is sometimes referred to as an exciplex laser. “Excimer” is short for “excited dimer,” while “exciplex” is short for “excited complex.” An excimer laser uses a mixture of a noble gas (argon, krypton, or xenon) and a halogen gas (fluorine or chlorine), which under suitable conditions of electrical stimulation and high pressure, emits coherent stimulated radiation (laser light) in the ultraviolet range. Furthermore, “exposure” in the present specification includes, unless otherwise specified, not only exposure by a mercury lamp, far ultraviolet rays represented by an excimer laser, X-rays, extreme ultraviolet rays (EUV light), or the like, but also writing by particle rays such as electron beams and ion beams.

As used herein, the term “hydrocarbon” refers to an organic compound or group having at least one carbon atom and at least one hydrogen atom; “alkyl” refers to a straight or branched chain saturated hydrocarbon group having the specified number of carbon atoms and having a valence of one; “alkylene” refers to an alkyl group having a valence of two; “hydroxyalkyl” refers to an alkyl group substituted with at least one hydroxyl group (—OH); “alkoxy” refers to “alkyl-O—”; “carboxyl” and “carboxylic acid group” refer to a group having the formula “—C(═O)—OH”; “cycloalkyl” refers to a monovalent group having one or more saturated rings in which all ring members are carbon; “cycloalkylene” refers to a cycloalkyl group having a valence of two; “alkenyl” refers to a straight or branched chain, monovalent hydrocarbon group having at least one carbon-carbon double bond; “alkenoxy” refers to “alkenyl-O—”; “alkenylene” refers to an alkenyl group having a valence of two; “cycloalkenyl” refers to a non-aromatic cyclic divalent hydrocarbon group having at least three carbon atoms, with at least one carbon-carbon double bond; “alkynyl” refers to a monovalent hydrocarbon group having at least one carbon-carbon triple bond; the term “aromatic group” refers to a monocyclic or polycyclic aromatic ring system that satisfies Huckel's Rule (4n+2 π electrons) and includes carbon atoms in the ring; the term “heteroaromatic group” refers to an aromatic group that includes one or more heteroatoms (e.g., 1-4 heteroatoms) selected from N, O, and S instead of a carbon atom in the ring; “aryl” refers to a monovalent monocyclic or polycyclic aromatic ring system where every ring member is carbon, and may include a group with an aromatic ring fused to at least one cycloalkyl or heterocycloalkyl ring; “arylene” refers to an aryl group having a valence of two; “alkylaryl” refers to an aryl group that has been substituted with an alkyl group; “arylalkyl” refers to an alkyl group that has been substituted with an aryl group; “aryloxy” refers to “aryl-O-”; and “arylthio” refers to “aryl-S—”.

The term “organic group” refers to a group containing at least one carbon atom (e.g., 1 to 60 carbon atoms) and may further contain a hydrogen atom, and may also contain nitrogen, oxygen, sulfur, silicon, a halogen atom, or the like. The organic group is not limited, and may be a hydrocarbon group or a derivative thereof. The derivative of the hydrocarbon group refers to a group that has one or more of N, O, S, Si, amide, sulfonyl, siloxane, carbonyl, carbonyloxy, or the like at the end or in the molecular chain of the hydrocarbon group. Other examples of an organic group include a cyano group.

The prefix “hetero” means that the compound or group includes at least one member that is a heteroatom (e.g., 1, 2, 3, or 4 or more heteroatom(s)) instead of a carbon atom, wherein the heteroatom(s) is each independently N, O, S, Si, or P; “heteroatom-containing group” refers to a substituent group that includes at least one heteroatom; “heteroalkyl” refers to an alkyl group having at least one heteroatom instead of carbon.

The term “heterocycloalkyl” refers to a cycloalkyl group having at least one heteroatom that is chosen independently from N, O, or S as a ring member instead of carbon; “heterocycloalkylene” refers to a heterocycloalkyl group having a valence of two. Exemplary 3-membered heterocycloalkyl groups containing 1 heteroatom include aziridinyl, oxiranyl, and thiiranyl. Exemplary 4-membered heterocycloalkyl groups containing 1 heteroatom include azetidinyl, oxetanyl, and thietanyl. Exemplary 5-membered heterocycloalkyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocycloalkyl groups containing 2 heteroatoms include dioxolanyl, oxathiolanyl, and dithiolanyl. Exemplary 5-membered heterocycloalkyl groups containing 3 heteroatoms include triazolinyl, oxadiazolinyl, and thiadiazolinyl. Exemplary 6-membered heterocycloalkyl groups containing 1 heteroatom include piperidinyl, tetrahydropyranyl, dihydropyridinyl, and thianyl. Exemplary 6-membered heterocycloalkyl groups containing 2 heteroatoms include piperazinyl, morpholinyl, dithianyl, and dioxanyl. Exemplary 6-membered heterocycloalkyl groups containing 2 heteroatoms include triazinanyl. Exemplary 7-membered heterocycloalkyl groups containing 1 heteroatom include azepanyl, oxepanyl, and thiepanyl. Exemplary 8-membered heterocycloalkyl groups containing 1 heteroatom include azocanyl, oxecanyl and thiocanyl. Exemplary bicyclic heterocycloalkyl groups include indolinyl, isoindolinyl, dihydrobenzofuranyl, dihydrobenzothienyl, tetrahydrobenzo-thienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, and 1,2,3,4-tetrahydro-1,6-naphthyridinyl.

The term “heteroaryl” means 4-8 membered monocyclic, 8-12 membered bicyclic, or 11-14 membered tricyclic aromatic ring systems having 1-4 heteroatoms (if monocyclic), 1-6 heteroatoms (if bicyclic), or 1-9 heteroatoms (if tricyclic) that are each independently selected from N, O, or S (e.g., carbon atoms and 1-3, 1-6, or 1-9 heteroatoms independently selected from N, O, or S, if monocyclic, bicyclic, or tricyclic, respectively). Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

The term “halogen” means a monovalent substituent that is fluorine (fluoro), chlorine (chloro), bromine (bromo), or iodine (iodo). The prefix “halo” means a group including one or more of a fluoro, chloro, bromo, or iodo substituent instead of a hydrogen atom. A combination of halo groups (e.g., bromo and fluoro), or only fluoro groups may be present. For example, the term “haloalkyl” refers to an alkyl group substituted with one or more halogens. As used herein, “substituted C₁₋₈ haloalkyl” refers to a C₁₋₈ alkyl group substituted with at least one halogen, and is further substituted with one or more other substituent groups that are not halogens. It is to be understood that substitution of a group with a halogen atom is not to be considered a heteroatom-containing group, because a halogen atom does not replace a carbon atom.

The term “fluorinated” means having one or more fluorine atoms incorporated into a group instead of hydrogen. For example, where a C₁₋₁₈ fluoroalkyl group is indicated, the fluoroalkyl group can include one or more fluorine atoms, for example, a single fluorine atom, two fluorine atoms (e.g., as a 1,1-difluoroethyl group), three fluorine atoms (e.g., as a 2,2,2-trifluoroethyl group), or fluorine atoms at each valence of carbon (e.g., as a perfluorinated group such as —CF₃, —C₂F₅, —C₃F₇, or —C₄F₉). A “substituted fluoroalkyl group” shall be understood to mean a fluoroalkyl group that is further substituted by at least one additional substituent group that does not contain fluorine atoms.

Each of the foregoing substituent groups optionally may be substituted unless expressly provided otherwise. The term “optionally substituted” refers to being substituted or unsubstituted. “Substituted” means that at least one hydrogen atom of the chemical structure or group is replaced with another terminal substituent group that is typically monovalent, provided that the designated atom's normal valence is not exceeded. When the substituent is oxo (i.e., O), then two geminal hydrogen atoms on the carbon atom are replaced with the terminal oxo group. It is further noted that the oxo group is bonded to carbon via a double bond to form a carbonyl (C═O), where the carbonyl group is represented herein as —C(O)—. Combinations of substituents or variables are permissible. Exemplary substituent groups that may be present on a “substituted” position include, but are not limited to, nitro (—NO₂), cyano (—CN), hydroxyl (—OH), oxo (O), amino (—NH₂), mono- or di-(C₁₋₆)alkylamino, alkanoyl (such as a C₂₋₆ alkanoyl group such as acyl), formyl (—C(O)H), carboxylic acid or an alkali metal or ammonium salt thereof, esters (including acrylates, methacrylates, and lactones) such as C₂₋₆ alkyl esters (—C(O)O-alkyl or —OC(O)-alkyl) and C₇₋₁₃ aryl esters (—C(O)O-aryl or —OC(O)-aryl); amido (—C(O)NR₂ wherein R is hydrogen or C₁₋₆ alkyl), carboxamido (—CH₂C(O)NR₂ wherein R is hydrogen or C₁₋₆ alkyl), halogen, thiol (—SH), C₁₋₆ alkylthio (—S-alkyl), thiocyano (—SCN), C₁₋₆ alkyl, C₂₋₆ alkenyl, C₂₋₆ alkynyl, C₁₋₆ haloalkyl, C₁₋₉ alkoxy, C₁₋₆ haloalkoxy, C₃₋₁₂ cycloalkyl, C₅₋₁₈ cycloalkenyl, C₂₋₁₈ heterocycloalkenyl, C₆₋₁₂ aryl having at least one aromatic ring (e.g., phenyl, biphenyl, naphthyl, or the like, each ring either substituted or unsubstituted aromatic), C₇₋₁₉ arylalkyl having 1 to 3 separate or fused rings and from 6 to 18 ring carbon atoms, arylalkoxy having 1 to 3 separate or fused rings and from 6 to 18 ring carbon atoms, C₇₋₁₂ alkylaryl, C₂₋₁₂ heterocycloalkyl, C₃₋₁₂ heteroaryl, C₁₋₆ alkyl sulfonyl (—S(O)₂-alkyl), C₆₋₁₂ arylsulfonyl (—S(O)₂-aryl), or tosyl (CH₃C₆H₄SO₂—). When a group is substituted, the indicated number of carbon atoms is the total number of carbon atoms in the group, excluding those of any substituents. For example, the group —CH₂CH₂CN is a cyano-substituted C₂ alkyl group.

As used herein, when a definition is not otherwise provided, a “divalent linking group” refers to a divalent group including one or more of —O—, —S—, —Te—, —Se—, —C(O)—, C(O)O—, —N(R′)—, —C(O)N(R′)—, —S(O)—, —S(O)₂—, —C(S)—, —C(Te)—, —C(Se)—, substituted or unsubstituted C₁₋₃₀ alkylene, substituted or unsubstituted C₃₋₃₀ cycloalkylene, substituted or unsubstituted C₂₋₃₀ heterocycloalkylene, substituted or unsubstituted C₆₋₃₀ arylene, substituted or unsubstituted C₃₋₃₀ heteroarylene, or a combination thereof, wherein each R′ is independently hydrogen, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl. Typically, the divalent linking group includes one or more of —O—, —S—, —C(O)—, —N(R′)—, —S(O)—, —S(O)₂—, substituted or unsubstituted C₁₋₃₀ alkylene, substituted or unsubstituted C₃₋₃₀ cycloalkylene, substituted or unsubstituted C₂₋₃₀ heterocycloalkylene, substituted or unsubstituted C₆₋₃₀ arylene, substituted or unsubstituted C₃₋₃₀ heteroarylene, or a combination thereof, wherein R′ is hydrogen, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl. More typically, the divalent linking group includes at least one of —O—, —C(O)—, —C(O)O—, —N(R′)—, —C(O)N(R′)—, substituted or unsubstituted C₁₋₁₀ alkylene, substituted or unsubstituted C₃₋₁₀ cycloalkylene, substituted or unsubstituted C₂₋₁₀ heterocycloalkylene, substituted or unsubstituted C₆₋₁₀ arylene, substituted or unsubstituted C₃₋₁₀ heteroarylene, or a combination thereof, wherein R is hydrogen, substituted or unsubstituted C₁₋₁₀ alkyl, substituted or unsubstituted C₁₋₁₀ heteroalkyl, substituted or unsubstituted C₆₋₁₀ aryl, or substituted or unsubstituted C₃₋₁₀ heteroaryl.

As used herein, an “acid-labile group” refers to a group in which a bond is cleaved by the action of an acid, optionally and typically with thermal treatment, resulting in formation of a polar group, such as a carboxylic acid or alcohol group. When this type of group is a pendant on a polymer, the formation of the polar group occurs on the polymer. Optionally and typically with a moiety connected to the cleaved bond becoming disconnected from the polymer. In other systems, a non-polymeric compound may include an acid-labile group that may be cleaved by the action of an acid, resulting in formation of a polar group, such as a carboxylic acid or alcohol group on a cleaved portion of the non-polymeric compound. Such acid is typically a photo-generated acid with bond cleavage occurring during post-exposure baking (PEB); however, embodiments are not limited thereto, and, for example, such acid may be thermally generated. Suitable acid-labile groups include, for example: tertiary alkyl ester groups, secondary or tertiary aryl ester groups, secondary or tertiary ester groups having a combination of alkyl and aryl groups, tertiary alkoxy groups, acetal groups, or ketal groups. Acid-labile groups are also commonly referred to in the art as “acid-cleavable groups,” “acid-cleavable protecting groups,” “acid-labile protecting groups,” “acid-leaving groups,” “acid-decomposable groups,” and “acid-sensitive groups.”

The term “unsaturated bond” refers to a double or triple bond. The term “unsaturated” or “partially unsaturated” refers to a moiety that includes at least one double or triple bond. The term “saturated” refers to a moiety that does not contain a double or triple bond, i.e., the moiety only contains single bonds.

As used herein, the term “(meth)acrylic” includes both acrylic and methacrylic species (i.e., acrylic and methacrylic monomers), and the term “(meth)acrylate” includes both acrylate and methacrylate species (i.e., acrylate and methacrylate monomers).

Embedded barrier layers (EBLs) for photolithography were typically designed and developed for use in ArF (193 nm) immersion formulation technologies. EBLs are formed using lower surface-energy materials that are added directly to the photoresist composition and can segregate to the surface of the resulting film during spin-coating. These materials may be developer switchable, or base-switchable polymers that can switch from hydrophobic to hydrophilic during the development step. The application of base-switchable polymer additives for defect reduction in formulations other than ArF photoresist compositions has created the need for development of base-switchable polymer additives to further improve photolithographic performance at other exposure wavelengths, such as KrF and/or EUV.

While fluorinated polymers may be useful additive polymers in immersion photoresists because they allow the formation of EBLs, they may be suitably replaced with more sustainable alternatives. Towards the goal of providing more sustainable alternative EBLs, the present inventors have developed a photoresist composition containing a barrier layer-forming additive polymer that does not rely on particular fluorine substitution.

An aspect provides a photoresist composition that includes a first polymer comprising a first repeating unit including a group of Formula (1) or (2); a second polymer including a first repeating unit including an acid-labile group; a photoacid generator; and a solvent. The first polymer includes a first repeating unit that includes a group of Formula (1) or (2):

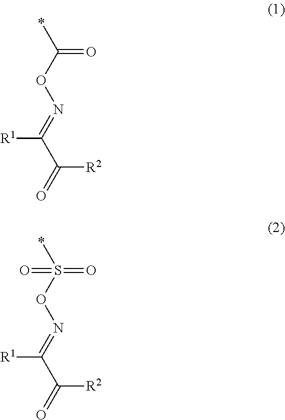

In Formulae (1) and (2), R¹ and R² are each independently hydrogen or an organic group. For example, R¹ and R² may each independently be hydrogen, substituted or unsubstituted C₁₋₃₀ alkyl, substituted or unsubstituted C₁₋₃₀ heteroalkyl, substituted or unsubstituted C₃₋₃₀ cycloalkyl, substituted or unsubstituted C₂₋₃₀ heterocycloalkyl, substituted or unsubstituted C₂₋₃₀ alkenyl, substituted or unsubstituted C₂₋₃₀ heteroalkenyl, substituted or unsubstituted C₆₋₃₀ aryl, or substituted or unsubstituted C₃₋₃₀ heteroaryl.

In Formulae (1) and (2), each of R¹ and R² may optionally further include one or more divalent linking groups as part of its structure. For example, each of R¹ and R² may further include as part of its structure one or more groups selected from —O—, —S—, —Si(R³)₂—, —N(R³)—, —S(O)—, —S(O)₂—, —C(O)—, —C(O)O—, —C(O)S—, —OC(O)O—, —N(R³)C(O)—, —C(O)N(R³)—, substituted or unsubstituted C₁₋₃₀ alkylene, substituted or unsubstituted C₃₋₃₀ cycloalkylene, substituted or unsubstituted C₂₋₃₀ heterocycloalkylene, substituted or unsubstituted C₆₋₃₀ arylene, substituted or unsubstituted C₃₋₃₀ heteroarylene, wherein each R³ is independently hydrogen, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl. For example, each of R¹ and R² may further include as part of its structure one or more groups selected from —O—, —S—, —Si(R³)₂—, —N(R³)—, —S(O)—, —S(O)₂—, —C(O)—, —C(O)O—, —C(O)S—, —OC(O)O—, —N(R³)C(O)—, or —C(O)N(R³)—.

In Formulae (1) and (2), R¹ and R² together optionally form a ring. The ring may be formed via one or more divalent linking groups. For example, R¹ and R² may together form a ring via one or more of —O—, —S—, —Te—, —Se—, —C(O)—, C(O)O—, —N(R⁴)—, —C(O)N(R⁴)—, —S(O)—, —S(O)₂—, —C(S)—, —C(Te)—, —C(Se)—, substituted or unsubstituted C₁₋₃₀ alkylene, substituted or unsubstituted C₃₋₃₀ cycloalkylene, substituted or unsubstituted C₂₋₃₀ heterocycloalkylene, substituted or unsubstituted C₆₋₃₀ arylene, substituted or unsubstituted C₃₋₃₀ heteroarylene, or a combination thereof, wherein each R⁴ is independently hydrogen, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl.

In Formulae (1) and (2), one of R¹ or R² is optionally bonded to a backbone of the first polymer. For example, R¹ or R² may be optionally bonded to a backbone of the first polymer via one or more divalent linking group selected from —O—, —S—, —Si(R⁵)₂—, —N(R⁵)—, —S(O)—, —S(O)₂—, —C(O)—, —C(O)O—, —C(O)S—, —OC(O)O—, —N(R⁵)C(O)—, —C(O)N(R⁵)—, substituted or unsubstituted C₁₋₃₀ alkylene, substituted or unsubstituted C₃₋₃₀ cycloalkylene, substituted or unsubstituted C₂₋₃₀ heterocycloalkylene, substituted or unsubstituted C₆₋₃₀ arylene, substituted or unsubstituted C₃₋₃₀ heteroarylene, wherein each R⁵ is independently hydrogen, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl.

In Formulae (1) and (2), * represents a point of bonding in the first repeating unit of the first polymer. As noted above, the R¹ or R² may be optionally bonded to the backbone of the first polymer. Hence, the group of Formulae (1) or (2) may be bonded to the backbone of the first polymer through any one or more of *, R¹, or R². Typically, the group of Formulae (1) or (2) is bonded to the backbone of the first polymer through *.

In some embodiments, the first repeating unit of the first polymer may include a group of Formula (1A) or (2A):

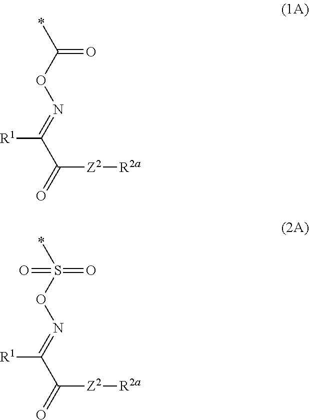

In Formulae (1A) and (2A), R^{1a} is hydrogen, substituted or unsubstituted C₁₋₃₀ alkyl, substituted or unsubstituted C₁₋₃₀ heteroalkyl, substituted or unsubstituted C₃₋₃₀ cycloalkyl, substituted or unsubstituted C₂₋₃₀ heterocycloalkyl, substituted or unsubstituted C₂₋₃₀ alkenyl, or substituted or unsubstituted C₂₋₃₀ heteroalkenyl.

In Formulae (1A) and (2A), R^{2a} is an organic group. For example, R^{2a} may be substituted or unsubstituted C₁₋₃₀ alkyl, substituted or unsubstituted C₁₋₃₀ heteroalkyl, substituted or unsubstituted C₃₋₃₀ cycloalkyl, substituted or unsubstituted C₂₋₃₀ heterocycloalkyl, substituted or unsubstituted C₂₋₃₀ alkenyl, substituted or unsubstituted C₂₋₃₀ heteroalkenyl, substituted or unsubstituted C₆₋₃₀ aryl, or substituted or unsubstituted C₃₋₃₀ heteroaryl.

In Formulae (1A) and (2A), Z² is NR′, O, or S, wherein R′ is hydrogen, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl. Typically, Z² is O.

In Formulae (1A) and (2A), R^{1a} and R^{2a} each optionally further includes one or more divalent linking groups as part of its structure. For example, R^{1a} and/or R^{2a} may further include as part of its structure one or more groups selected from —O—, —S—, —Si(R³)₂—, —N(R³)—, —S(O)—, —S(O)₂—, —C(O)—, —C(O)O—, —C(O)S—, —OC(O)O—, —N(R³)C(O)—, —C(O)N(R³)—, substituted or unsubstituted C₁₋₃₀ alkylene, substituted or unsubstituted C₃₋₃₀ cycloalkylene, substituted or unsubstituted C₂₋₃₀ heterocycloalkylene, substituted or unsubstituted C₆₋₃₀ arylene, substituted or unsubstituted C₃₋₃₀ heteroarylene, wherein each R³ is independently hydrogen, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl.

In Formulae (1A) and (2A), one of R^{1a} or R^{2a} is optionally bonded to a backbone of the first polymer. For example, R^{1a} or R^{2a} may be optionally bonded to a backbone of the first polymer via one or more divalent linking group selected from —O—, —S—, —Si(R⁵)₂—, —N(R⁵)—, —S(O)—, —S(O)₂—, —C(O)—, —C(O)O—, —C(O)S—, —OC(O)O—, —N(R⁵)C(O)—, —C(O)N(R⁵)—, substituted or unsubstituted C₁₋₃₀ alkylene, substituted or unsubstituted C₃₋₃₀ cycloalkylene, substituted or unsubstituted C₂₋₃₀ heterocycloalkylene, substituted or unsubstituted C₆₋₃₀ arylene, substituted or unsubstituted C₃₋₃₀ heteroarylene, wherein each R⁵ is independently hydrogen, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl.

In Formulae (1A) and (2A), * represents a point of bonding in the first repeating unit of the first polymer. As noted above, R^{1a} or R^{2a} may be optionally bonded to the backbone of the first polymer. Hence, the group of Formulae (1A) or (2A) may be bonded to the backbone of the first polymer through any one or more of *, R^{1a}, or R^{2a}. Typically, the group of Formulae (1A) or (2A) is bonded to the backbone of the first polymer through *.

In still other embodiments, the first repeating unit of the first polymer may include a group of Formula (1B) or (2B):

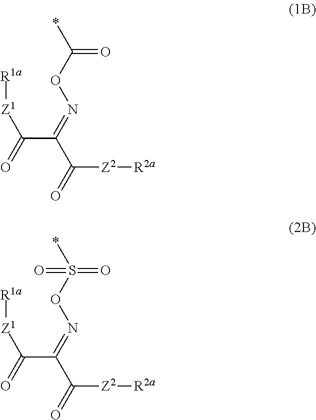

In Formulae (1B) and (2B), R^{1a} is hydrogen, substituted or unsubstituted C₁₋₃₀ alkyl, substituted or unsubstituted C₁₋₃₀ heteroalkyl, substituted or unsubstituted C₃₋₃₀ cycloalkyl, substituted or unsubstituted C₂₋₃₀ heterocycloalkyl, substituted or unsubstituted C₂₋₃₀ alkenyl, or substituted or unsubstituted C₂₋₃₀ heteroalkenyl. For example, R^{1a} may be substituted or unsubstituted C₁₋₃₀ alkyl, substituted or unsubstituted C₁₋₃₀ heteroalkyl, substituted or unsubstituted C₃₋₃₀ cycloalkyl, substituted or unsubstituted C₂₋₃₀ heterocycloalkyl, substituted or unsubstituted C₂₋₃₀ alkenyl, or substituted or unsubstituted C₂₋₃₀ heteroalkenyl

In Formulae (1B) and (2B), R^{2a} is an organic group. For example, R^{2a} may be substituted or unsubstituted C₁₋₃₀ alkyl, substituted or unsubstituted C₁₋₃₀ heteroalkyl, substituted or unsubstituted C₃₋₃₀ cycloalkyl, substituted or unsubstituted C₂₋₃₀ heterocycloalkyl, substituted or unsubstituted C₂₋₃₀ alkenyl, substituted or unsubstituted C₂₋₃₀ heteroalkenyl, substituted or unsubstituted C₆₋₃₀ aryl, or substituted or unsubstituted C₃₋₃₀ heteroaryl.

In Formulae (1B) and (2B), Z¹ and Z² are each independently a single bond, NR′, O, or S wherein R′ is hydrogen, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl, provided that at least one of Z¹ or Z² is NR′, O, or S.

In Formulae (1B) and (2B), R^{1a} and R^{2a} each optionally further includes one or more divalent linking groups as part of its structure. For example, R^{1a} and/or R^{2a} may further include as part of its structure one or more groups selected from —O—, —S—, —Si(R³)₂—, —N(R³)—, —(O)—, —S(O)₂—, —C(O)—, —C(O)O—, —C(O)S—, —OC(O)O—, —N(R³)C(O)—, —C(O)N(R³)—, substituted or unsubstituted C₁₋₃₀ alkylene, substituted or unsubstituted C₃₋₃₀ cycloalkylene, substituted or unsubstituted C₂₋₃₀ heterocycloalkylene, substituted or unsubstituted C₆₋₃₀ arylene, substituted or unsubstituted C₃₋₃₀ heteroarylene, wherein each R³ is independently hydrogen, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl.

In Formulae (1B) and (2B), one of R^{1a} or R^{2a} is optionally bonded to a backbone of the first polymer. For example, R^{1a} or R^{2a} may be optionally bonded to a backbone of the first polymer via one or more divalent linking group selected from —O—, —S—, —Si(R⁵)₂—, —N(R⁵)—, —S(O)—, —S(O)₂—, —C(O)—, —C(O)O—, —C(O)S—, —OC(O)O—, —N(R⁵)C(O)—, —C(O)N(R⁵)—, substituted or unsubstituted C₁₋₃₀ alkylene, substituted or unsubstituted C₃₋₃₀ cycloalkylene, substituted or unsubstituted C₂₋₃₀ heterocycloalkylene, substituted or unsubstituted C₆₋₃₀ arylene, substituted or unsubstituted C₃₋₃₀ heteroarylene, wherein each R⁵ is independently hydrogen, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl.

In Formulae (1B) and (2B), * represents a point of bonding in the first repeating unit of the first polymer. As noted above, R^{1a} or R^{2a} may be optionally bonded to the backbone of the first polymer. Hence, the group of Formulae (1B) or (2B) may be bonded to the backbone of the first polymer through any one or more of *, R^{1a}, or R^{2a}. Typically, the group of Formulae (1B) or (2B) is bonded to the backbone of the first polymer through *.

In some embodiments, the first repeating unit of the first polymer is formed from a monomer that includes an ethylenically unsaturated double bond. As used herein, an “ethylenically unsaturated double bond” refers to a vinyl-containing polymerizable group, and typically may be selected from substituted or unsubstituted C₂₋₂₀ alkenyl, substituted or unsubstituted norbornene, substituted or unsubstituted (meth)acrylic, substituted or unsubstituted vinyl ether, substituted or unsubstituted vinyl ketone, substituted or unsubstituted vinyl ester, or substituted or unsubstituted vinyl aromatic.

In some embodiments, the first polymer is free of —CF₂— and —CF₃ groups that are bonded to a carbon atom. In some embodiments, the first polymer is free of fluorine. It should be understood that “free of —F, —CF₃, or —CF₂— groups” means that the first polymer excludes groups such as —CH₂CF₃ and —CH₂CF₂CH₃.

Non-limiting exemplary monomers for preparing first repeating units that include groups of Formula (1) or (2) include the following:

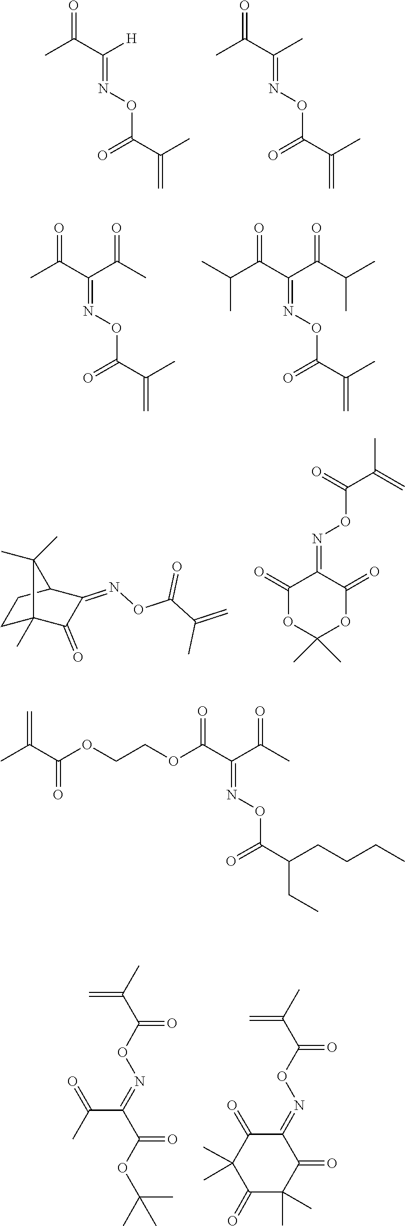

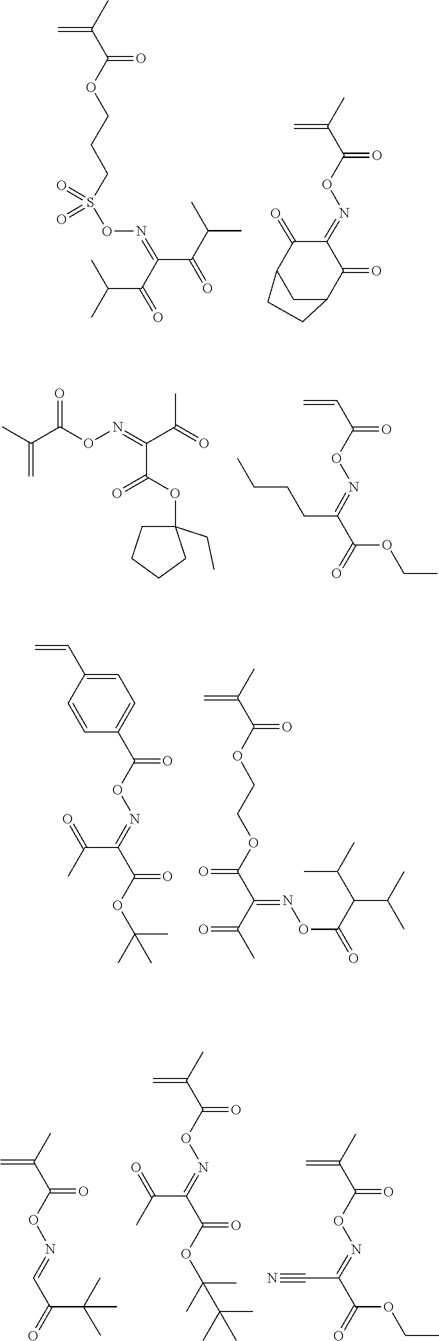

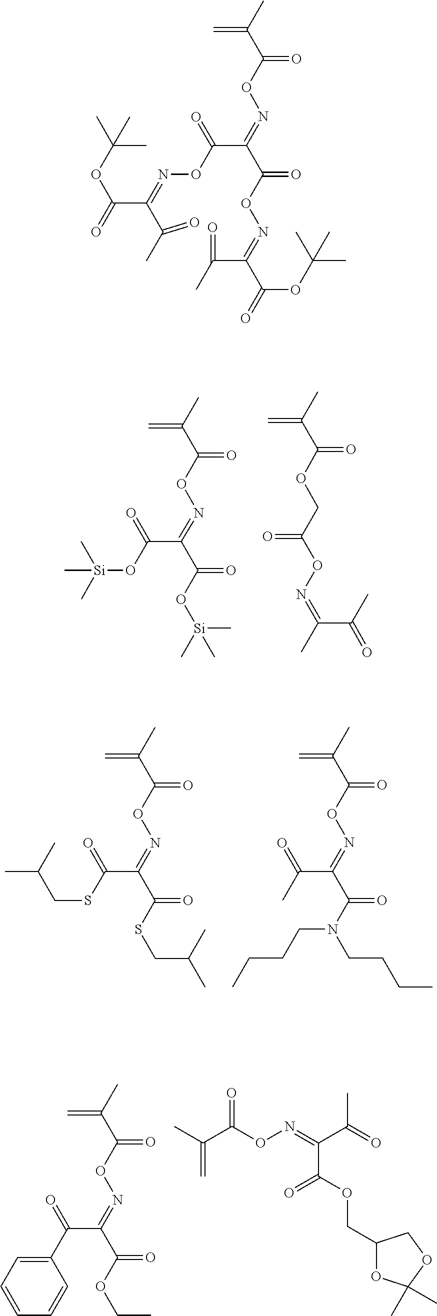

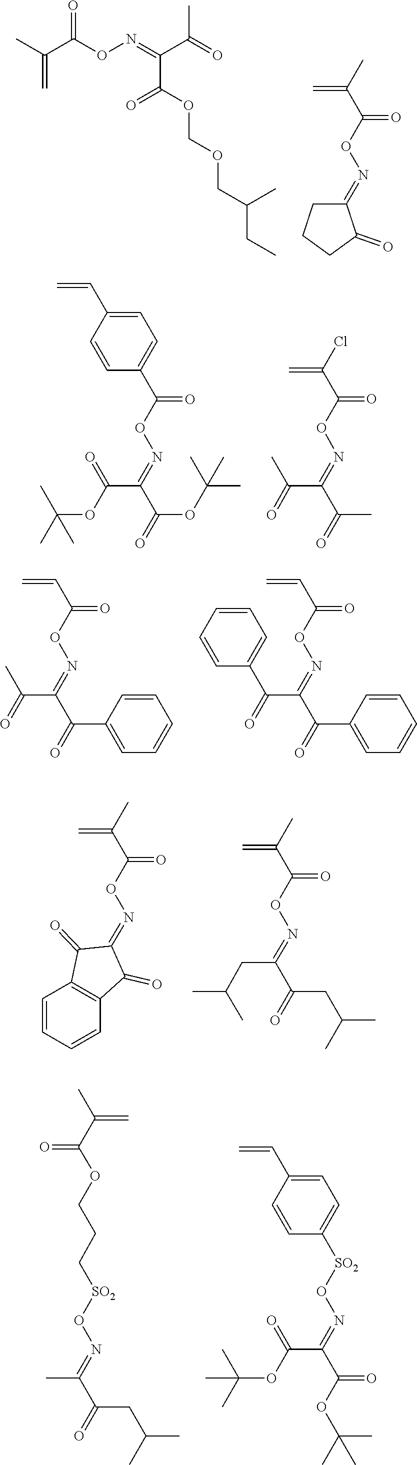

A total content of all repeating units including a group of Formula (1) or Formula (2) in the first polymer is from 30 to 100 mol % based on total repeating units of the first polymer. For example, the total content of all repeating units including a group of Formula (1) or Formula (2) in the first polymer is from 40 to 100 mol %, or from 50 to 100 mol %, or from 60 to 100 mol % based on total repeating units of the first polymer.

The first polymer may optionally further include one or more additional repeating units different from the first repeating unit. For example, the first polymer may optionally include one or more additional repeating units as described below. The one or more additional units if present in the first polymer may be used in an amount of up to 70 mol %, and typically from 3 to 50 mol %, based on total repeating units in the first polymer.

The first polymer is typically present in the photoresist composition in an amount from 0.1 to 20 weight percent (wt %) based on total solids of the photoresist composition. For example, the first polymer may be present in the photoresist composition in an amount from 0.1 to 10 wt %, or from 5 to 15 wt %, each based on total solids of the photoresist composition.

The first polymer typically has a weight average molecular weight (M_{w}) from 1,000 to 50,000 Dalton (Da), preferably from 2,000 to 30,000 Da, more preferably 4,000 to 25,000 Da, and still more preferably from 5,000 to 25,000 Da. The polydispersity index (PDI) of the first polymer, which is the ratio of M_{w} to number average molecular weight (Mₙ) is typically from 1.1 to 3, and more typically from 1.1 to 2. Molecular weight values are determined by gel permeation chromatography (GPC) using polystyrene standards.

The first polymer may be prepared using any suitable method(s) in the art. For example, one or more monomers corresponding to the repeating units described herein may be combined, or fed separately, using suitable solvent(s) and initiator, and polymerized in a reactor. For example, the first polymer may be obtained by polymerization of the respective monomers under any suitable conditions, such as by heating at an effective temperature, irradiation with actinic radiation at an effective wavelength, or a combination thereof.

The photoresist composition also includes a second polymer comprising a first repeating unit comprising an acid-labile group. Suitable acid-labile groups include, for example, tertiary alkyl ester groups, secondary or tertiary aryl ester groups, secondary or tertiary ester groups having a combination of alkyl and aryl groups, tertiary alkoxy groups, acetal groups, ketal groups, tertiary carbonate groups, and tertiary carbamate groups. Typically, the acid-labile group may be an acetal group, a ketal group, a tertiary carbonate group, a tertiary carbamate group, or a tertiary ester group. As used herein, the “tertiary carbamate group” includes tertiary carbamate ester groups having alkyl groups, tertiary carbamate ester groups having aryl groups, and tertiary carbamate ester groups having a combination of alkyl and aryl groups. As used herein, the “tertiary carbonate group” includes tertiary carbonate ester groups having alkyl groups, tertiary carbonate ester groups having aryl groups, and tertiary carbonate ester groups having a combination of alkyl and aryl groups. Preferably, the acid-labile group includes a tertiary ester group.

In one or more embodiments, the second polymer may include a first repeating unit including an acid-labile repeating unit that is derived from a monomer represented by one or more of Formulae (3), (4), (5), (6), or (7):

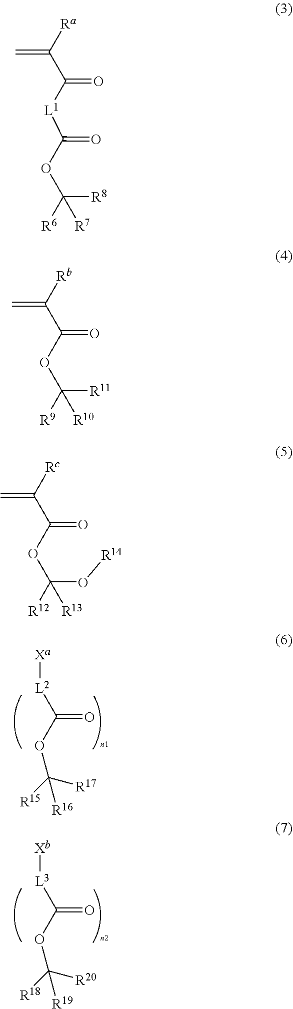

In Formulae (3), (4) and (5), R^{a}, R^{b}, and R^{c} may each independently be hydrogen, deuterium, halogen, cyano, substituted or unsubstituted C₁₋₁₀ alkyl, or substituted or unsubstituted C₃₋₆ cycloalkyl. Preferably, R^{a}, R^{b}, and R^{c} may each independently be hydrogen, fluorine, or substituted or unsubstituted C₁₋₅ alkyl, typically methyl.

In Formula (3), L¹ is a divalent linking group. For example, L¹ may include 1 to 10 carbon atoms and at least one heteroatom. In a typical example, L¹ may be —OCH₂—, —OCH₂CH₂O—, or —N(R^{3a})—, wherein R^{3a} is hydrogen or C₁₋₆ alkyl.

In Formulae (3) and (4), R⁶ to R¹¹ are each independently hydrogen, substituted or unsubstituted C₁₋₂₀ alkyl, substituted or unsubstituted C₃₋₂₀ cycloalkyl, substituted or unsubstituted C₂₋₂₀ heterocycloalkyl, substituted or unsubstituted C₂₋₂₀ alkenyl, substituted or unsubstituted C₃₋₂₀ cycloalkenyl, substituted or unsubstituted C₃₋₂₀ heterocycloalkenyl, substituted or unsubstituted C₆₋₂₀ aryl, or substituted or unsubstituted C₃₋₂₀ heteroaryl, provided that no more than one of R⁶ to R⁸ may be hydrogen, and that no more than one of R⁹ to R¹¹ may be hydrogen, and provided that if one of R⁶ to R⁸ is hydrogen, then at least one of the others from R⁶ to R⁸ is substituted or unsubstituted C₆₋₂₀ aryl or substituted or unsubstituted C₃₋₂₀ heteroaryl, and if one of R⁹ to R¹¹ is hydrogen, then at least one of the others from R⁹ to R¹¹ is substituted or unsubstituted C₆₋₂₀ aryl or substituted or unsubstituted C₃₋₂₀ heteroaryl. Preferably, R⁶ to R¹¹ are each independently substituted or unsubstituted C₁₋₆ alkyl or substituted or unsubstituted C₃₋₁₀ cycloalkyl. Each of R⁶ to R¹¹ may optionally further comprise a divalent linking group as part of their structure.

For example, any one or more of R⁶ to R¹¹ may be independently a group of the formula —CH₂C(O)CH₍₃₋ₙ₎Yₙ, or —CH₂C(O)OCH₍₃₋ₙ₎Yₙ, where each Y is independently substituted or unsubstituted C₂₋₁₀ heterocycloalkyl and n is 1 or 2. For example, each Y may be independently substituted or unsubstituted C₂₋₁₀ heterocycloalkyl including a group of the formula —O(C^{a1})(C^{a2})O—, wherein C^{a1} and C^{a2} are each independently hydrogen or substituted or unsubstituted alkyl, and where C^{a1} and C^{a2} together optionally form a ring.

In Formula (3), any two of R⁶ to R⁸ together optionally may form a ring, which may further include a divalent linking group as part of its structure, and wherein the ring may be substituted or unsubstituted. In Formula (4), any two of R⁹ to R¹¹ together optionally may form a ring, which may further include a divalent linking group as part of its structure, wherein the ring group may be substituted or unsubstituted.

In Formulae (5) and (7), R¹², R¹³, R¹⁸, and R¹⁹ each independently may be hydrogen, substituted or unsubstituted C₁₋₂₀ alkyl, substituted or unsubstituted C₃₋₂₀ cycloalkyl, substituted or unsubstituted C₂₋₂₀ heterocycloalkyl, substituted or unsubstituted C₆₋₂₀ aryl, or substituted or unsubstituted C₃₋₂₀ heteroaryl; and R¹⁴ and R²⁰ are each independently substituted or unsubstituted C₁₋₂₀ alkyl, substituted or unsubstituted C₃₋₂₀ cycloalkyl, or substituted or unsubstituted C₂₋₂₀ heterocycloalkyl. Preferably, R¹², R¹³, R¹⁸, and R¹⁹ each independently may be hydrogen, substituted or unsubstituted C₁₋₂₀ alkyl, substituted or unsubstituted C₃₋₂₀ cycloalkyl, or substituted or unsubstituted C₂₋₂₀ heterocycloalkyl. Each of R¹², R¹³, R¹⁸, and R¹⁹ may optionally further comprise a divalent linking group as part of their structure.

In Formula (5), any two of R¹² to R¹⁴ together optionally may form a ring, which may further include a divalent linking group as part of its structure, wherein the ring group may be substituted or unsubstituted.

In Formula (6), R¹⁵ to R¹⁷ may be each independently be substituted or unsubstituted C₁₋₂₀ alkyl, substituted or unsubstituted C₃₋₂₀ cycloalkyl, substituted or unsubstituted C₂₋₂₀ heterocycloalkyl, substituted or unsubstituted C₆₋₂₀ aryl, or substituted or unsubstituted C₃₋₂₀ heteroaryl, provided that no more than one of R¹⁵ to R¹⁷ may be hydrogen and provided that if one of R¹⁵ to R¹⁷ is hydrogen, then at least one of the others from R¹⁵ to R¹⁷ is substituted or unsubstituted C₆₋₂₀ aryl or substituted or unsubstituted C₃₋₂₀ heteroaryl. Each of R¹⁵ to R¹⁷ may optionally further comprise a divalent linking group as part of their structure.

For example, any one or more of R¹⁵ to R¹⁷ may be independently a group of the formula —CH₂C(O)CH₍₃₋ₙ₎Yₙ, or —CH₂C(O)OCH₍₃₋ₙ₎Yₙ, where each Y is independently substituted or unsubstituted C₂₋₁₀ heterocycloalkyl and n is 1 or 2. For example, each Y may be independently substituted or unsubstituted C₂₋₁₀ heterocycloalkyl including a group of the formula —O(C^{a1})(C^{a2})O—, wherein C^{a1} and C^{a2} are each independently hydrogen or substituted or unsubstituted alkyl, and where C^{a1} and C^{a2} together optionally form a ring.

In Formula (6), any two of R¹⁵ to R¹⁷ together optionally form a ring, which may further include a divalent linking group as part of its structure, wherein the ring group may be substituted or unsubstituted.

In Formulae (6) and (7), X^{a} and X^{b} are each independently a polymerizable group comprising an ethylenically unsaturated double bond, preferably (meth)acrylate or C₂ alkenyl.

In Formulae (6) and (7), L² and L³ are each independently a single bond or a divalent linking group, provided that L² is not a single bond when X^{a} is C₂ alkenyl and that L³ is not a single bond when X^{b} is C₂ alkenyl. Preferably, L² and L³ are each independently substituted or unsubstituted C₆₋₃₀ arylene or substituted or unsubstituted C₆₋₃₀ cycloalkylene.

In Formulae (6) and (7), n1 is 0 or 1 and n2 is 0 or 1. It is to be understood that when n1 is 0, the L² group is connected directly to the oxygen atom. It is to be understood that when n2 is 0, the L³ group is connected directly to the oxygen atom.

In Formula (7), any two of R¹⁸ to R²⁰ together optionally may form a ring, which may further include a divalent linking group as part of its structure, wherein the ring group may be substituted or unsubstituted.

In some aspects, each of R⁶ to R²⁰ optionally may further include as part of their structure one or more divalent linking groups selected from —O—, —C(O)—, —C(O)O—, —S—, —S(O)₂—, —N(R′)—, or —C(O)N(R′)—, wherein R′ may be hydrogen, substituted or unsubstituted C₁₋₂₀ alkyl, substituted or unsubstituted C₃₋₂₀ cycloalkyl, or substituted or unsubstituted C₂₋₂₀ heterocycloalkyl.

Non-limiting examples of repeating units having an acid labile group include the following:

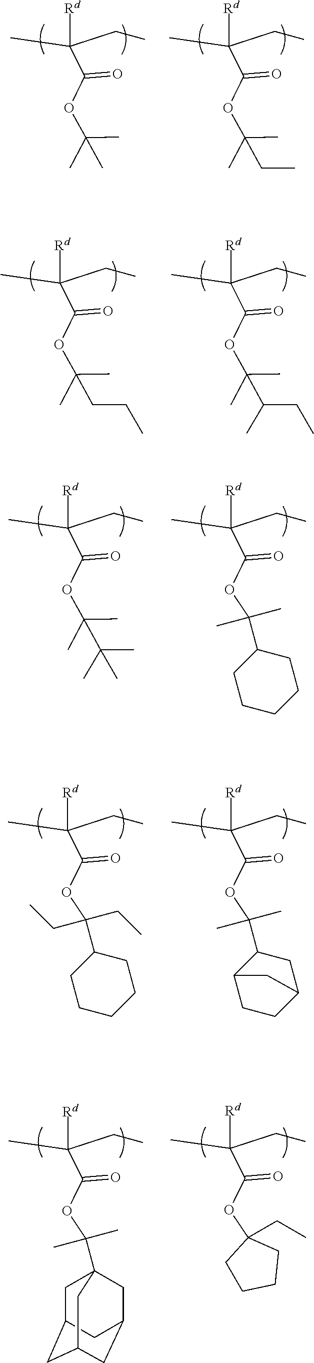

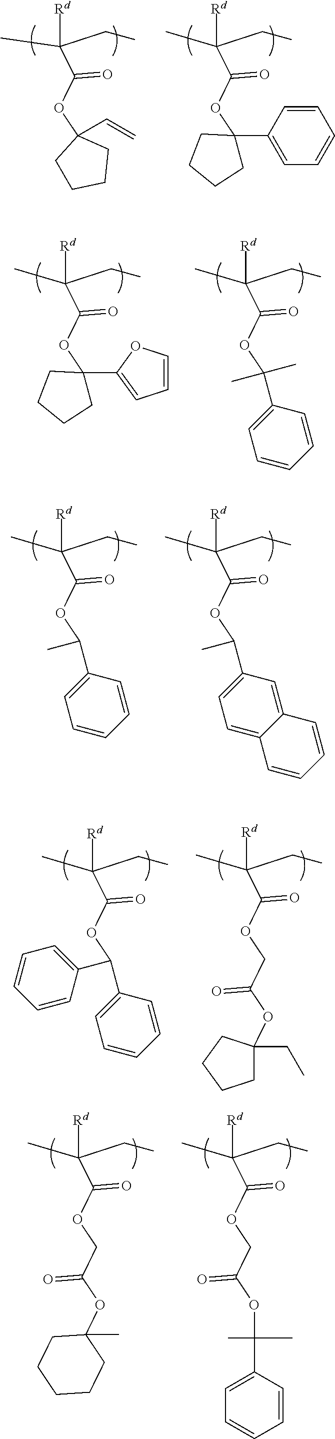

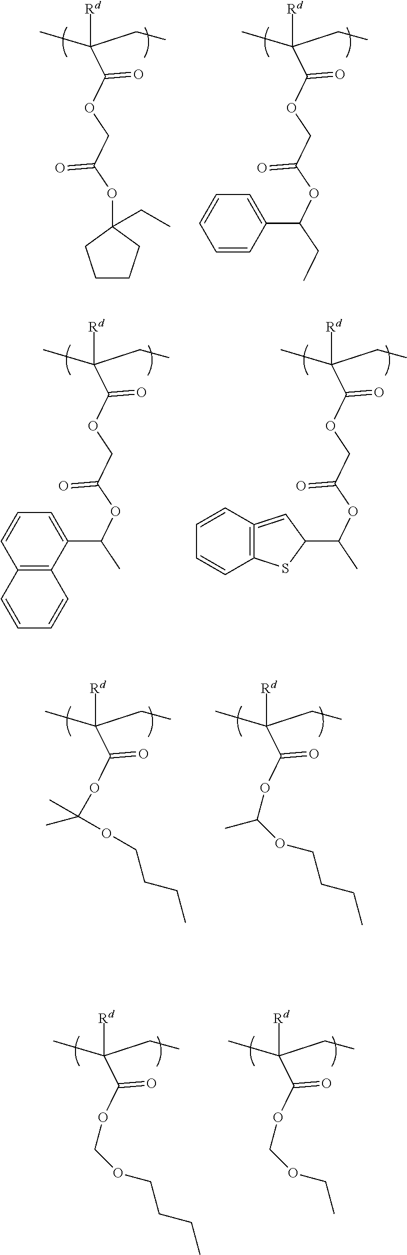

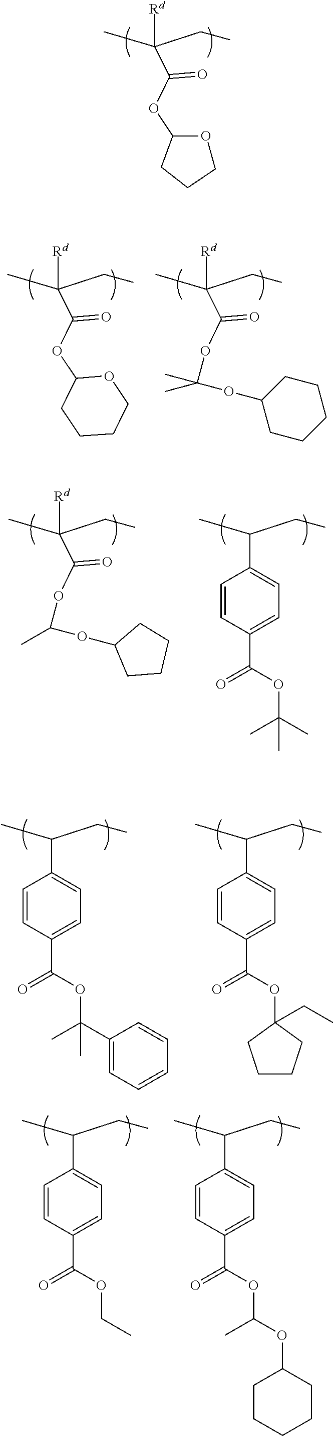

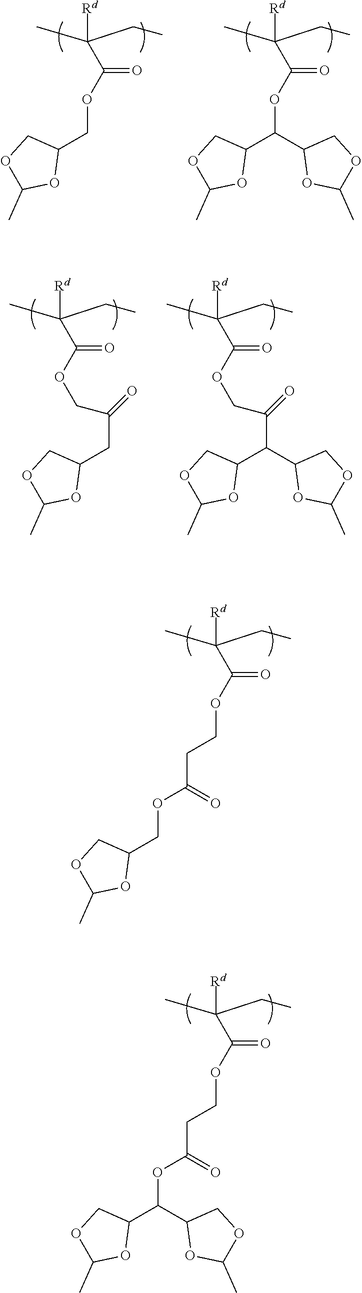

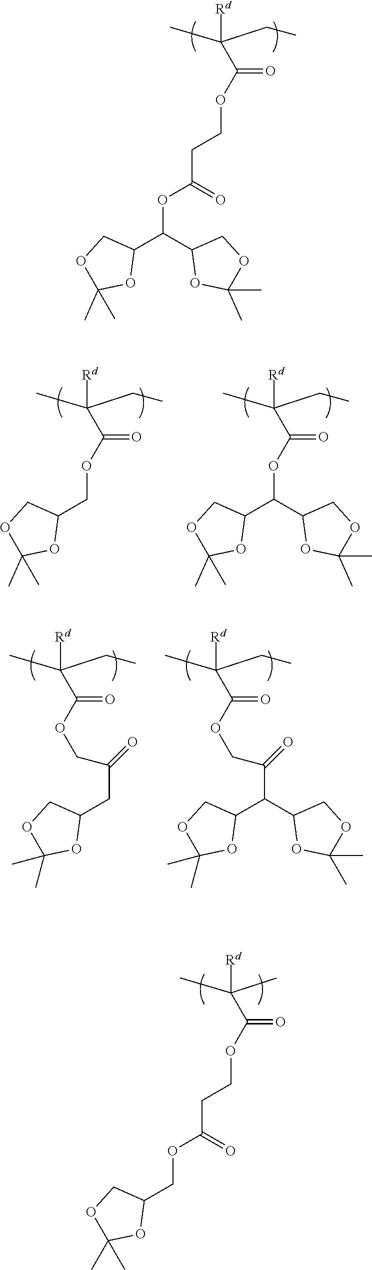

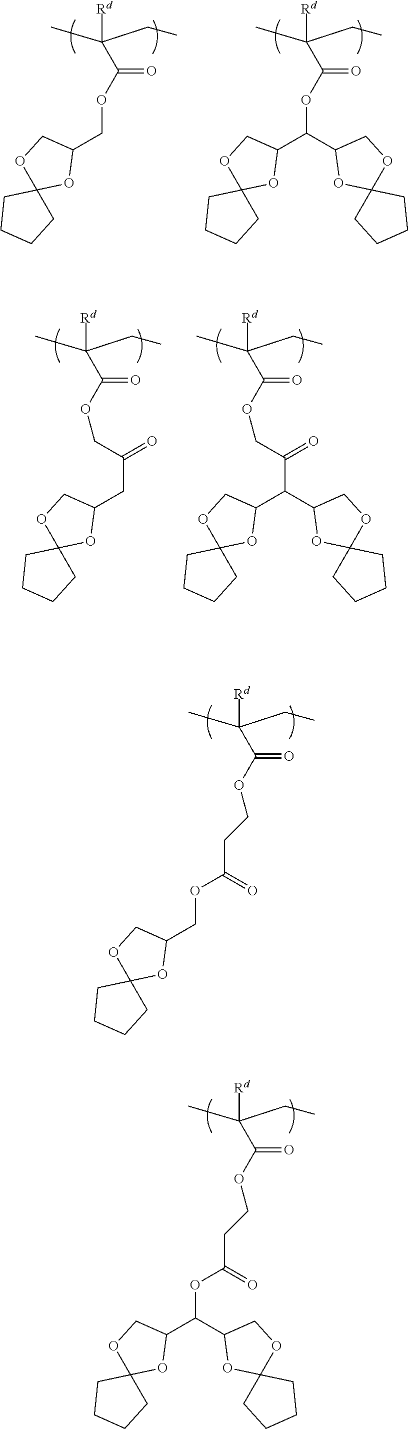

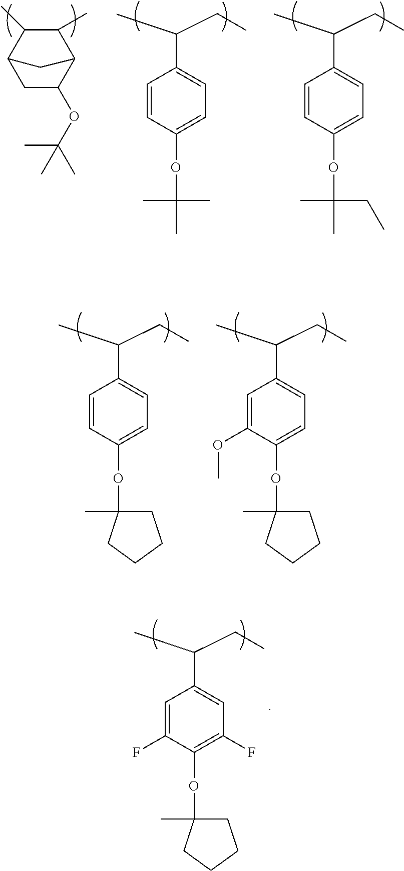

wherein R^{d} is hydrogen, deuterium, halogen, substituted or unsubstituted C₁₋₆ alkyl, or substituted or unsubstituted C₃₋₆ cycloalkyl.

A total content of all repeating units including acid-labile group in the second polymer is from 5 to 100 mol % based on total repeating units of the second polymer. For example, the total content of all repeating units including an acid-labile group in the second polymer is from 15 to 100 mol %, or from 15 to 80 mol %, or from 30 to 60 mol % based on total repeating units of the second polymer.

The second polymer may optionally further include one or more additional repeating units different from the first repeating unit. For example, the second polymer may optionally include one or more additional repeating units as described below. The one or more additional units if present in the second polymer may be used in an amount of up to 80 mol %, and typically from 3 to 60 mol %, based on total repeating units in the second polymer.

The second polymer is typically present in the photoresist composition in an amount from 0.1 to 80 weight percent (wt %) based on total solids of the photoresist composition. For example, the second polymer may be present in the photoresist composition in an amount from 0.1 to 75 wt %, or from 15 to 70 wt %, each based on total solids of the photoresist composition.

The second polymer typically has a M_{w} from 1,000 to 50,000 Da, preferably from 2,000 to 30,000 Da, more preferably 4,000 to 25,000 Da, and still more preferably from 5,000 to 25,000 Da. The PDI of the second polymer is typically from 1.1 to 3, and more typically from 1.1 to 2. Molecular weight values are determined by GPC using polystyrene standards.

The second polymer may be prepared using any suitable method(s) in the art. For example, one or more monomers corresponding to the repeating units described herein may be combined, or fed separately, using suitable solvent(s) and initiator, and polymerized in a reactor. For example, the second polymer may be obtained by polymerization of the respective monomers under any suitable conditions, such as by heating at an effective temperature, irradiation with actinic radiation at an effective wavelength, or a combination thereof.

The first polymer and/or the second polymer may further include one or more additional repeating units besides the first repeating unit as described above. The additional repeating units may be, for example, one or more units for purposes of adjusting properties of the composition, such as etch rate and solubility. Exemplary repeating units may include those derived from one or more of (meth)acrylate, vinyl aromatic, vinyl ether, vinyl ketone, and/or vinyl ester monomers. The first polymer and the second polymer of the composition may be a homopolymer or a copolymer that includes two or more structurally different repeating units.

In some aspects, the first polymer and/or the second polymer may further include a repeating unit comprising a polar group, where the polar group is pendant to the backbone of the respective polymer. For example, the polar group can be a lactone group, a sultone group, a hydroxy aryl group, a carboxylic acid group, or a combination thereof, but embodiments are not limited thereto.

In one or more embodiments, the first polymer and/or the second polymer may further include a repeating unit derived from one or more monomers of Formula (8):

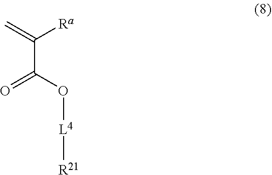

wherein R^{a} is hydrogen, deuterium, halogen, cyano, substituted or unsubstituted C₁₋₁₀ alkyl, or substituted or unsubstituted C₃₋₆ cycloalkyl.

In Formula (8), L⁴ is a single bond or one or more divalent linking groups. Exemplary divalent linking groups for L⁴ include one or more of substituted or unsubstituted C₁₋₃₀ alkylene, substituted or unsubstituted C₁₋₃₀ heteroalkylene, substituted or unsubstituted C₃₋₃₀ cycloalkylene, substituted or unsubstituted C₂₋₃₀ heterocycloalkylene, substituted or unsubstituted C₆₋₃₀ arylene, substituted or unsubstituted C₃₋₃₀ heteroarylene, —O—, —C(O)—, —C(O)O—, —S—, —S(O)₂—, —N(R^{8a})—, or —C(O)N(R^{8b})—, wherein R^{8a} and R^{8b} may be each independently hydrogen, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl.

It is to be understood that when L⁴ is a single bond, the moiety —R²¹ is directly connected to the oxygen atom adjacent to the carbonyl group (i.e., —C(O)O—R²¹).

In Formula (8), R²¹ is a substituted or unsubstituted C₄₋₂₀ lactone-containing group or a substituted or unsubstituted C₄₋₂₀ sultone-containing group. The C₄₋₂₀ lactone-containing group and the C₄₋₂₀ sultone-containing group may be monocyclic, polycyclic, or fused polycyclic.

Exemplary monomers of Formula (8) may include one or more of the following:

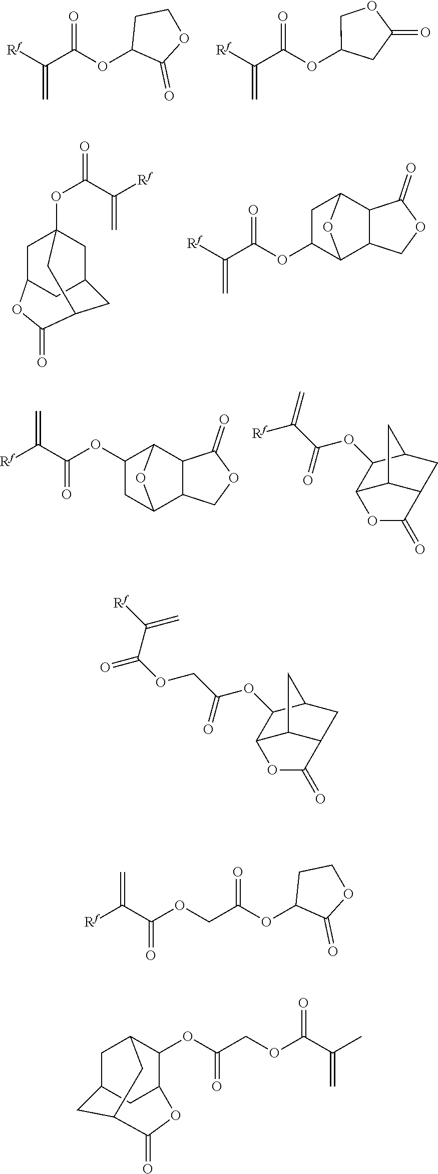

wherein R^{f} is as defined for R^{a} in Formula (8).

In some aspects, the repeating unit comprising a polar group may be a repeating unit that is base-soluble and/or that has a pKa of less than or equal to 12. For example, such repeating units may include one or more of Formulae (9), (10), or (11):

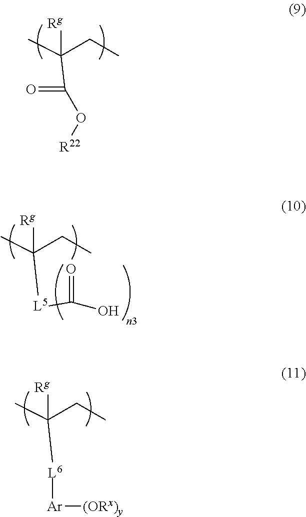

wherein each R^{g} may be hydrogen, deuterium, halogen, cyano, substituted or unsubstituted C₁₋₁₀ alkyl, or substituted or unsubstituted C₃₋₆ cycloalkyl. Preferably, R^{g} may be hydrogen, fluorine, or substituted or unsubstituted C₁₋₅ alkyl, typically methyl.

In Formula (9), R²² may be substituted or unsubstituted C₁₋₁₀₀ or C₁₋₂₀ alkyl, typically C₁₋₁₂ alkyl; substituted or unsubstituted C₃₋₃₀ or C₃₋₂₀ cycloalkyl; or substituted or unsubstituted poly(C₁₋₃ alkylene oxide). Preferably, the substituted C₁₋₁₀₀ or C₁₋₂₀ alkyl, the substituted C₃₋₃₀ or C₃₋₂₀ cycloalkyl, and the substituted poly(C₁₋₃ alkylene oxide) are substituted with one or more of halogen, a fluoroalkyl group such as a C₁₋₄ fluoroalkyl group, typically fluoromethyl, a sulfonamide group —NH—S(O)₂—Y¹ where Y¹ is F or C₁₋₄ perfluoroalkyl (e.g., —NHSO₂CF₃), or a fluoroalcohol group (e.g., —C(CF₃)₂OH).

In Formula (10), L⁵ represents a single bond or a multivalent linking group chosen, for example, from optionally substituted aliphatic, such as C₁₋₆ alkylene or C₃₋₂₀ cycloalkylene, and aromatic hydrocarbons, and combinations thereof, optionally with one or more linking moieties chosen from —O—, —C(O)—, —C(O)O—, —S—, —S(O)₂—, —NR^{10a}—, or —C(O)N(R^{10a})— wherein R^{10a} is chosen from hydrogen, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl. For example, the first and/or second polymer may further include a repeating unit derived from one or more monomers of Formula (10) wherein L⁵ is a single bond or a multivalent linking group selected from substituted or unsubstituted C₁₋₂₀ alkylene, typically C₁₋₆ alkylene; substituted or unsubstituted C₃₋₂₀ cycloalkylene; typically, C₃₋₁₀ cycloalkylene; and substituted or unsubstituted C₆₋₂₄ arylene.

In Formula (10), n3 is an integer from 1 to 5, typically 1. It is to be understood that when n3 is 1, the group L⁵ is a divalent linking group. It is to be understood that when n3 is 2, the group L⁵ is a trivalent linking group. Similarly, it is to be understood that when n3 is 3, the group L⁵ is a tetravalent linking group; when n3 is 4, the group L⁵ is a pentavalent linking group; and when n3 is 5, the group L⁵ is a hexavalent linking group. Accordingly, in the context of Formula (10), the term “multivalent linking group” refers to any of a divalent, trivalent, tetravalent, pentavalent, and/or hexavalent linking groups. In some aspects, when n3 is 2 or greater, the carboxylic acid groups (—C(O)OH) may be connected to the same atom of the linking group L⁵. In other aspects, when n3 is 2 or greater, the carboxylic acid groups (—C(O)OH) may be connected to different atoms of the linking group L⁵.

In Formula (11), L⁶ represents a single bond or one or more divalent linking groups. Preferably, L⁶ may be a single bond, substituted or unsubstituted C₆₋₃₀ arylene, or substituted or unsubstituted C₆₋₃₀ cycloalkylene.

In Formula (11), Ar is a substituted C₅₋₆₀ aromatic group that optionally includes one or more aromatic ring heteroatoms chosen from N, O, S, or a combination thereof, wherein the aromatic group may be monocyclic, non-fused polycyclic, or fused polycyclic. When the C₅₋₆₀ aromatic group is polycyclic, the ring or ring groups may be fused (such as naphthyl or the like), non-fused, or a combination thereof. When the polycyclic C₅₋₆₀ aromatic group is non-fused, the ring or ring groups may be directly linked (such as biaryls, biphenyl, or the like) or may be bridged by a heteroatom (such as triphenylamino or diphenylene ether). In some aspects, the polycyclic C₅₋₆₀ aromatic group may include a combination of fused rings and directly linked rings (such as binaphthyl or the like).

In Formula (11), y may be an integer from 1 to 12, preferably from 1 to 6, and typically from 1 to 3. Each RX may independently be hydrogen or methyl.

Non-limiting examples of repeating units of Formulae (9), (10), and/or (11) include one or more of the following:

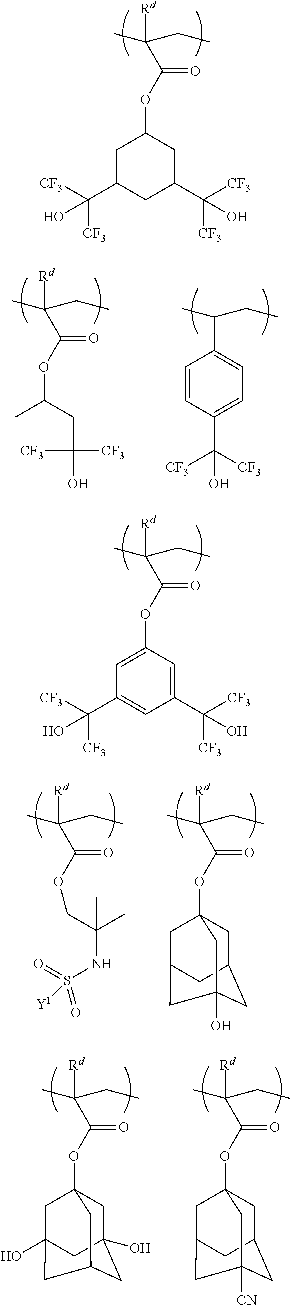

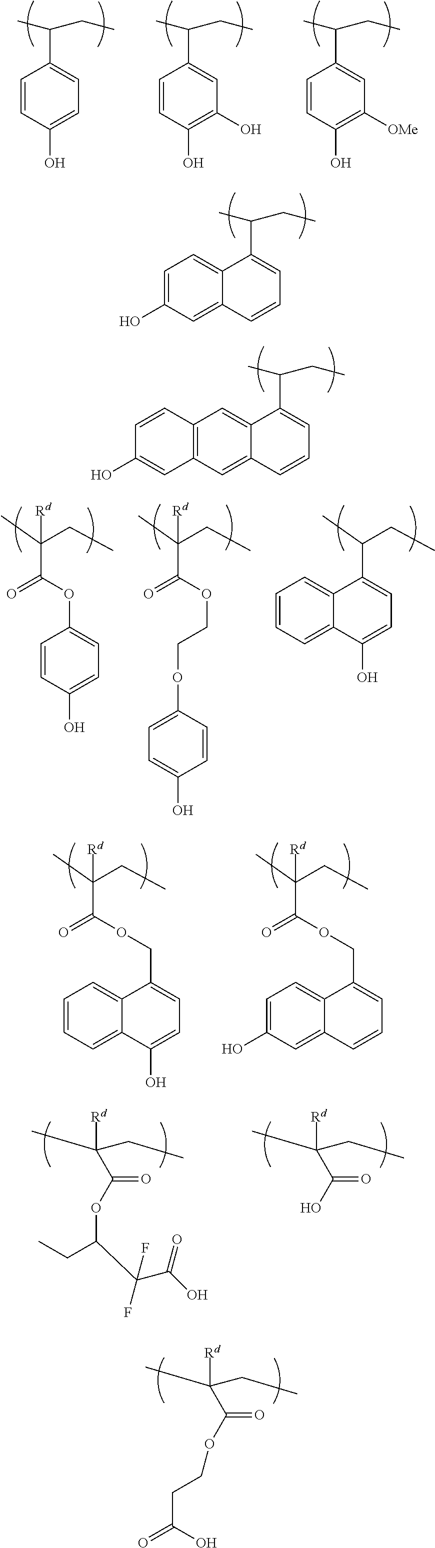

wherein R^{d} may be hydrogen, deuterium, halogen, cyano, substituted or unsubstituted C₁₋₁₀ alkyl, or substituted or unsubstituted C₃₋₆ cycloalkyl. Preferably, R^{d} may be hydrogen, fluorine, or substituted or unsubstituted C₁₋₅ alkyl, typically methyl.

When present, the first polymer and/or the second polymer typically includes a repeating unit comprising a polar group (pendant to a backbone of the polymer) in an amount from 1 to 60 mol %, typically from 5 to 50 mol %, more typically from 5 to 40 mol %, based on total repeating units in the polymer.

A repeating unit of the first polymer and/or the second polymer may include a salt group. As used herein, a “salt group” refers to a moiety having a positive charge and/or a negative charge, such as having a positively charged or negatively charged moiety that is bonded pendant to the backbone of the respective polymer. The repeating unit of the polymer including a salt group may comprise a photoacid generator (PAG) group or a photo-decomposable quencher (PDQ) group. For example, a repeating unit of the first polymer and/or the second polymer may include a salt group that is represented by Formula (12a) or (12b):

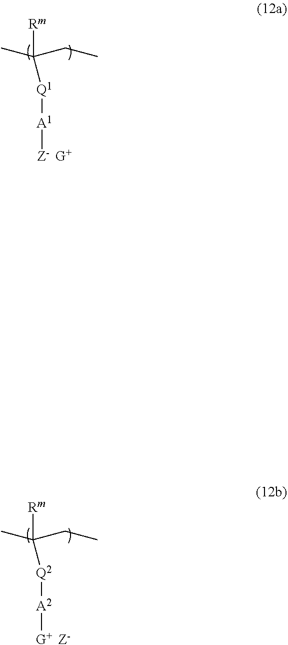

In Formulae (12a) and (12b), each R^{m} may be hydrogen, deuterium, halogen, cyano, substituted or unsubstituted C₁₋₁₀ alkyl, or substituted or unsubstituted C₃₋₆ cycloalkyl. Preferably, R^{m} is hydrogen, fluorine, or substituted or unsubstituted C₁₋₅ alkyl, typically methyl.

In Formulae (12a) and (12b), Q¹ and Q² may each be independently a single bond or a divalent linking group. Preferably, Q¹ and Q² may each independently include 1 to 10 carbon atoms and at least one heteroatom, more preferably —C(O)—O—.

In Formulae (12a) and (12b), A¹ and A² may be each independently one or more of substituted or unsubstituted C₁₋₃₀ alkylene, substituted or unsubstituted C₃₋₃₀ cycloalkylene, substituted or unsubstituted C₂₋₃₀ heterocycloalkylene, substituted or unsubstituted C₆₋₃₀ arylene, or substituted or unsubstituted C₃₋₃₀ heteroarylene. In some embodiments, A¹ and A² may be each independently a divalent C₁₋₃₀ perfluoroalkylene group that is optionally substituted.

In Formula (12a), Z⁻ is an anionic moiety that is bonded to A¹, the conjugated acid of which typically has a pKa from −15 to 10. Z may be a sulfonate, a carboxylate, an anion of a sulfonamide, an anion of a sulfonimide, or a methide anion.

In Formula (12a), G⁺ is an organic cation as defined herein. In some embodiments, G⁺ is an iodonium cation substituted with two alkyl groups, two aryl groups, or a combination of alkyl and aryl groups; or a sulfonium cation substituted with three alkyl groups, three aryl groups, or a combination of alkyl and aryl groups.

In Formula (12b), Z⁻ is an anion compound as defined herein, the conjugated acid of which typically has a pKa from −15 to 10. Z may be a sulfonate, a carboxylate, an anion of a sulfonamide, an anion of a sulfonimide, or a methide anion species.

In Formula (12b), G⁺ is an organic cation that is bonded to A². For example, G⁺ may include an iodonium cation substituted with two alkyl groups, two aryl groups, or a combination of alkyl and aryl groups; or a sulfonium cation substituted with three alkyl groups, three aryl groups, or a combination of alkyl and aryl groups.

In still other embodiments, when the first polymer and/or the second polymer includes a repeating unit having a salt group, the respective polymer may include a zwitterionic species. For example, the first polymer and/or the second polymer may include repeating units having a salt group of the Formula (12c):

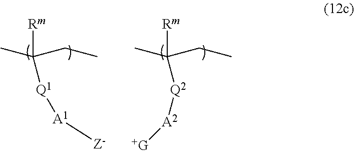

wherein, in Formula (12c), each R^{m} is independently as defined for Formulae (12a) and (12b).

In Formula (12c), Q¹, A¹, and Z are as defined in Formula (12a), and Q², A², and G⁺ are as defined in Formula (12b).

Exemplary repeating units of Formula (12a) include the following:

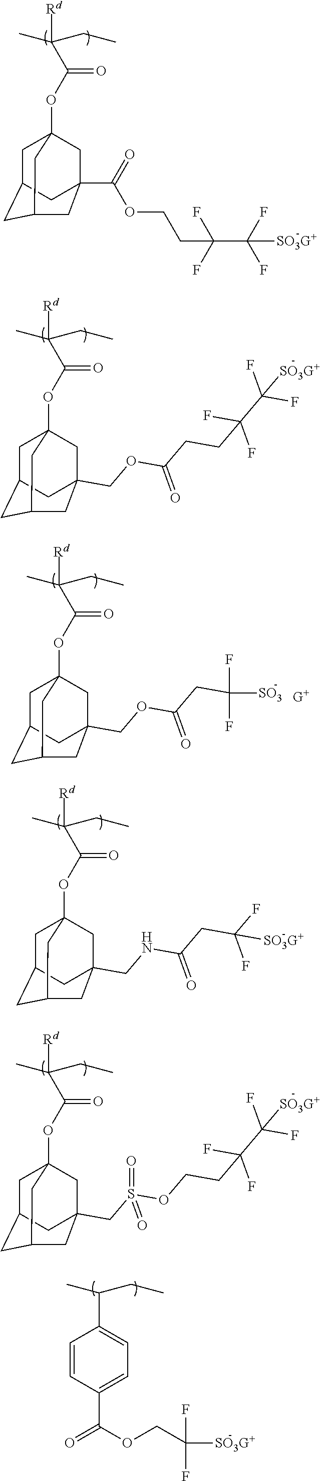

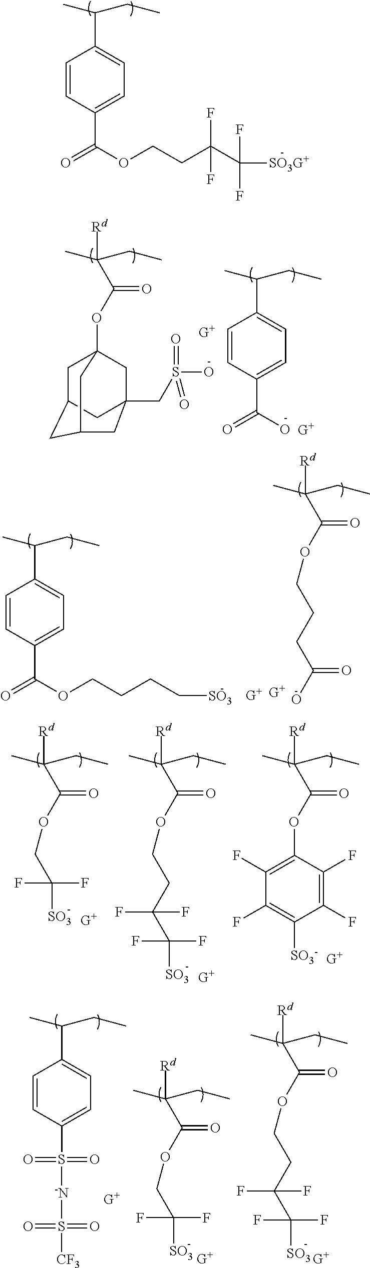

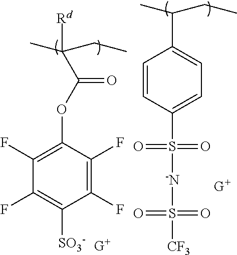

wherein G⁺ is the organic cation, and each R^{d} is independently as defined for R^{m} in Formulae (12a).

Exemplary repeating units of Formula (12b) include the following:

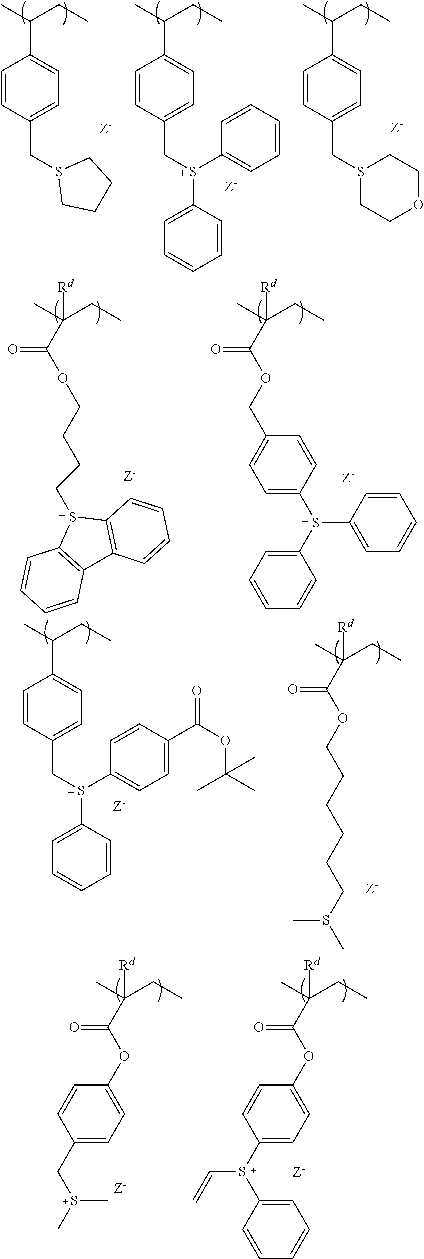

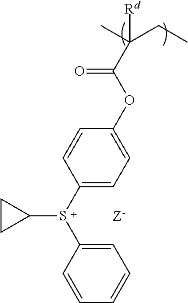

wherein Z⁻ is an anion group as defined herein, and each R^{d} is independently as defined for R^{m} in Formulae (12b).

The repeating unit of the first polymer and/or the second polymer including a salt typically may be present in an amount from 1 to 35 mol %, typically from 1 to 25 mol %, more typically from 2 to 15 mol %, based on total repeating units of the respective polymer.

The first polymer and/or the second polymer may include a repeating unit that includes a base-labile group. As referred to herein, base-labile groups are functional groups that can undergo cleavage reaction to provide polar groups such as hydroxyl, carboxylic acid, sulfonic acid, and the like, in the presence of an aqueous alkaline developer after exposure and post-exposure baking steps. The base-labile group will not react significantly (e.g., will not undergo a bond-breaking reaction) prior to a development step of the photoresist composition that comprises the base-labile group. Thus, for instance, a base-labile group will be substantially inert during pre-exposure soft-bake, exposure, and post-exposure bake steps. By “substantially inert” it is meant that ≤5%, typically ≤1%, of the base-labile groups (or moieties) will decompose, cleave, or react during the pre-exposure soft-bake, exposure, and post-exposure bake steps. The base-labile group is reactive under typical photoresist development conditions using, for example, an aqueous alkaline photoresist developer such as a 0.26 normal (N) aqueous solution of tetramethylammonium hydroxide (TMAH). For example, a 0.26 N aqueous solution of TMAH may be used for single puddle development or dynamic development, e.g., where the 0.26 N TMAH developer is dispensed onto an imaged photoresist layer for a suitable time such as 10 to 120 seconds (s). An exemplary base-labile group is an ester group, typically a fluorinated ester group. In some cases, the polymer including the base-labile group is substantially not miscible with and has a lower surface energy than the first polymer and other solid components of the composition. When coated on a substrate, the polymer including the base-labile group can thereby segregate from other solid components of the composition to a top surface of the formed photoresist layer.

The base-labile repeating unit may be derived from one or more monomers of formula (13):

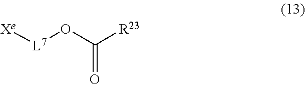

In Formula (13), X^{e} is a polymerizable group, typically selected from substituted or unsubstituted C₂₋₂₀ alkenyl or substituted or unsubstituted (meth)acryloyl.

In Formula (13), L⁷ is a divalent linking group that may include, for example, one or more of substituted or unsubstituted C₁₋₂₀ alkylene, substituted or unsubstituted C₃₋₂₀ cycloalkylene, —C(O)—, or —C(O)O—.

In Formula (13), R²³ is substituted or unsubstituted C₁₋₂₀ fluoroalkyl, provided that the carbon atom bonded to the carbonyl (C═O) in Formula (13) is substituted with at least one fluorine atom.

Exemplary repeating units derived from monomers of formula (13) may include the following:

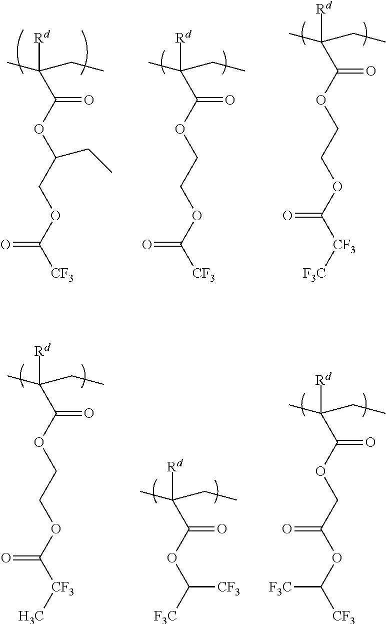

wherein R^{d} may be hydrogen, deuterium, halogen, cyano, substituted or unsubstituted C₁₋₁₀ alkyl, or substituted or unsubstituted C₃₋₆ cycloalkyl. Preferably, R^{d} may be hydrogen, fluorine, or substituted or unsubstituted C₁₋₅ alkyl, typically methyl.

The first polymer and/or the second polymer may include a repeating unit including two or more base-labile groups. For example, the respective polymer can include a repeating unit derived from one or more monomers of formula (14):

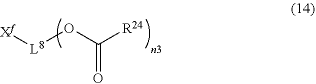

In Formula (14), X^{f} is a polymerizable group as defined for X^{e}.

In Formula (14), R²⁴ is substituted or unsubstituted C₁₋₂₀ fluoroalkyl, provided that the carbon atom bonded to the carbonyl (C═O) in Formula (14) is substituted with at least one fluorine atom.

In Formula (14), L⁸ is a polyvalent linking group including one or more of substituted or unsubstituted C₁₋₂₀ alkylene, substituted or unsubstituted C₃₋₂₀ cycloalkylene, —C(O)—, or —C(O)O—.

In Formula (14), n3 is an integer of 2 or greater, for example 2 or 3.

Exemplary repeating units derived from monomers of Formula (14) may include the following:

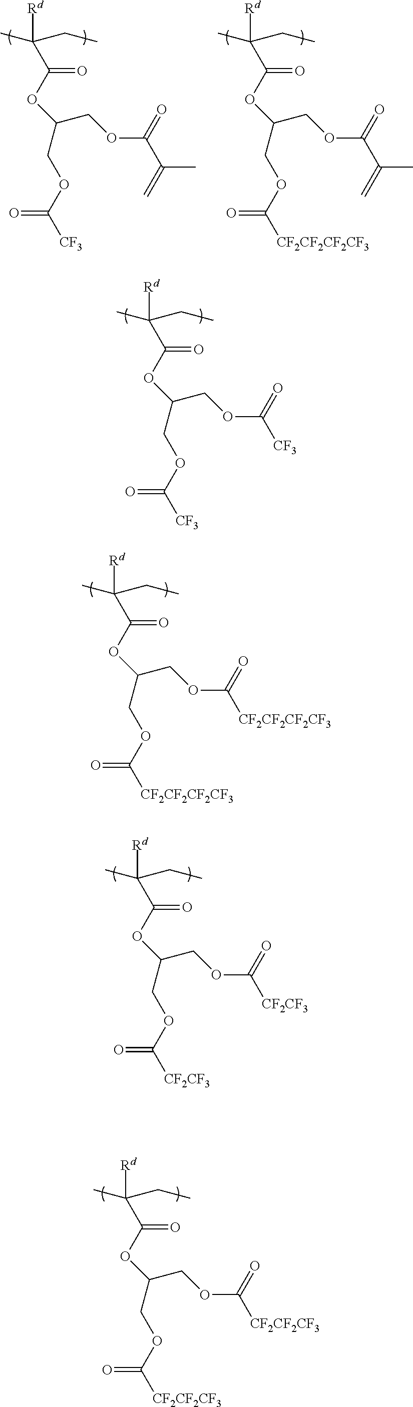

wherein R^{d} may be hydrogen, deuterium, halogen, cyano, substituted or unsubstituted C₁₋₁₀ alkyl, or substituted or unsubstituted C₃₋₆ cycloalkyl. Preferably, R^{d} may be hydrogen, fluorine, or substituted or unsubstituted C₁₋₅ alkyl, typically methyl.

For example, the first polymer and/or the second polymer can include a repeating unit derived from one or more monomers of Formula (15):

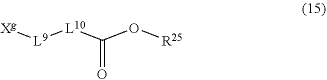

In Formula (15), X^{g} is a polymerizable group as defined for X^{e}.

In Formula (15), R²⁵ is substituted or unsubstituted C₁₋₂₀ alkyl.

In Formula (15), L⁹ is a divalent linking group.

In Formula (15), L¹⁰ is substituted or unsubstituted C₁₋₂₀ fluoroalkylene wherein the carbon atom bonded to the carbonyl (C═O) in Formula (15) is substituted with at least one fluorine atom.

Exemplary repeating units derived from monomers of Formula (15) may include the following:

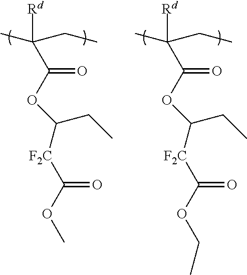

wherein R^{d} may be hydrogen, deuterium, halogen, cyano, substituted or unsubstituted C₁₋₁₀ alkyl, or substituted or unsubstituted C₃₋₆ cycloalkyl. Preferably, R^{d} may be hydrogen, fluorine, or substituted or unsubstituted C₁₋₅ alkyl, typically methyl.

When present, the repeating unit having a base-labile group may be present in the first polymer and/or the second polymer in an amount from 1 to 95 mol %, more typically from 1 to 80 mol %, still more typically from 10 to 50 mol %, based on total repeating units in the respective polymer.

Non-limiting exemplary second polymers of the present invention include one or more of the following:

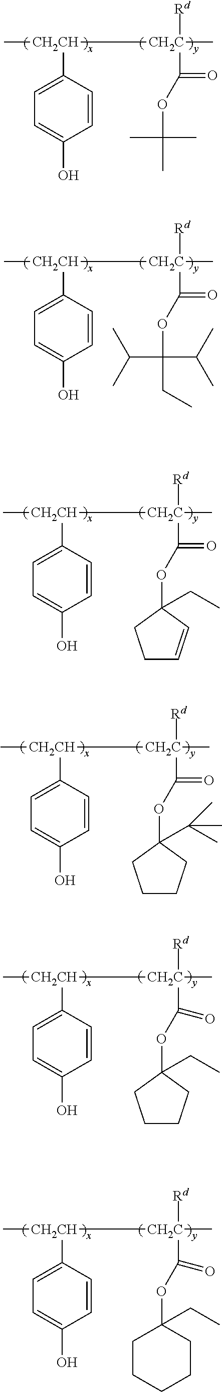

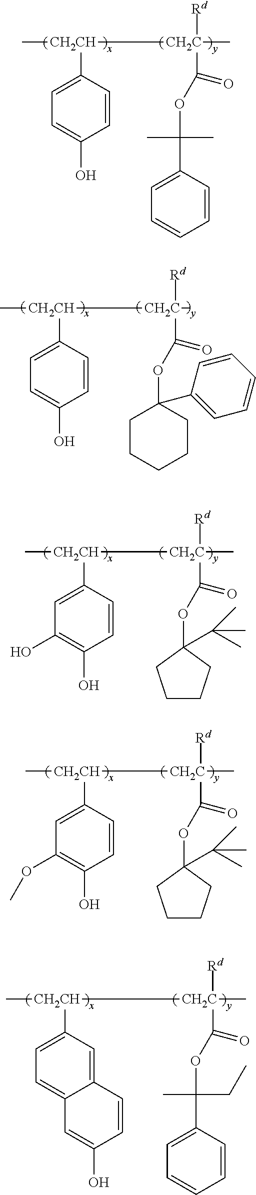

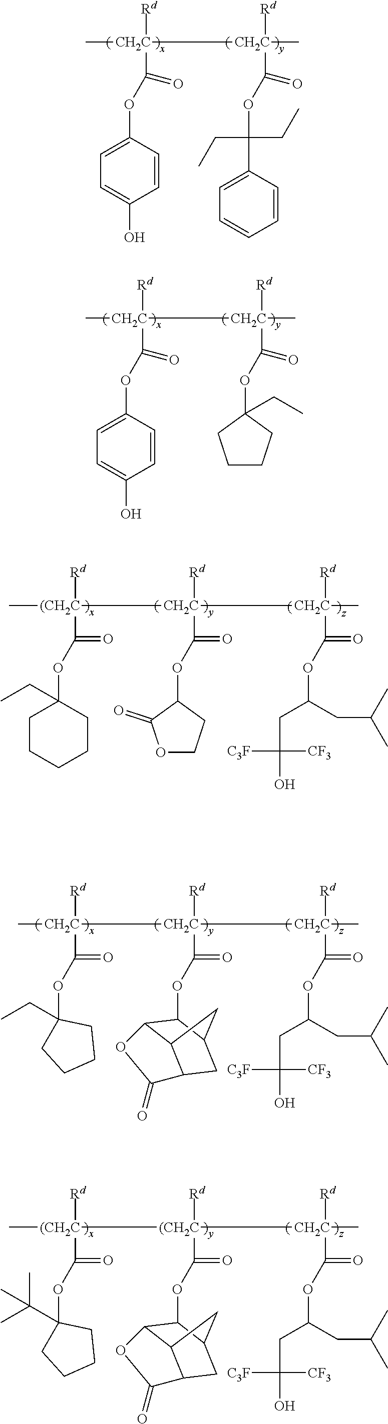

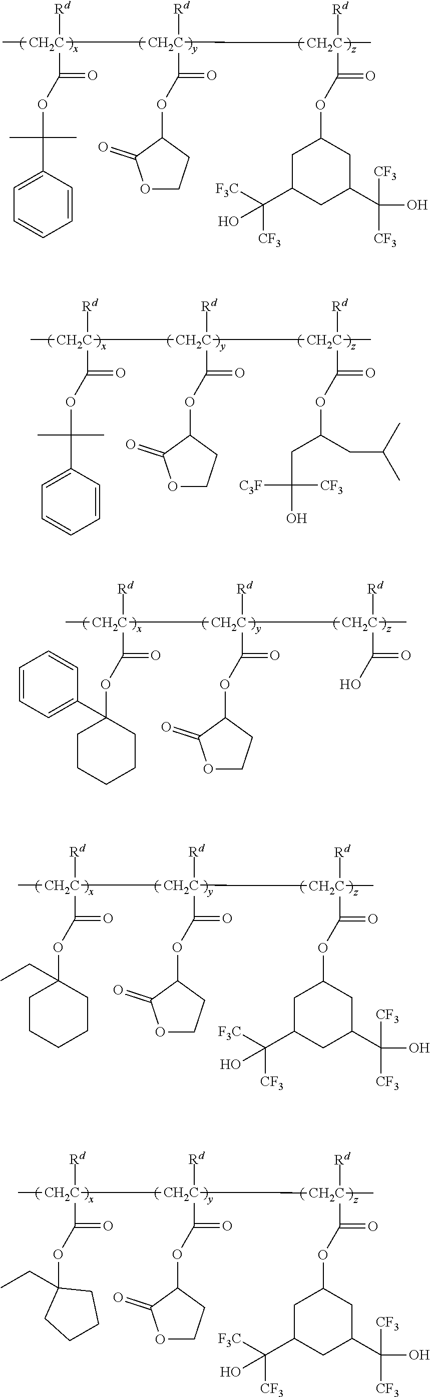

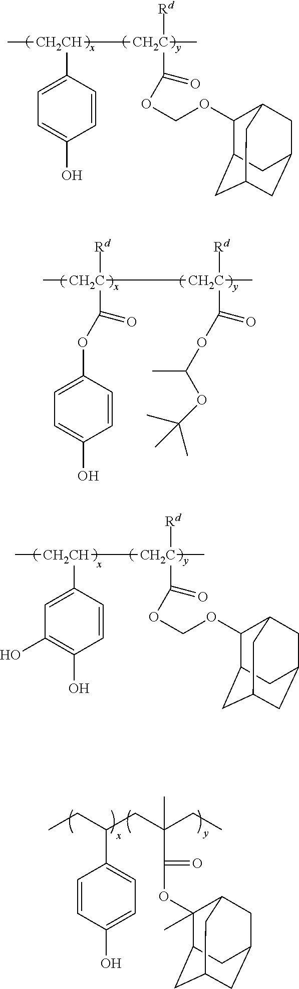

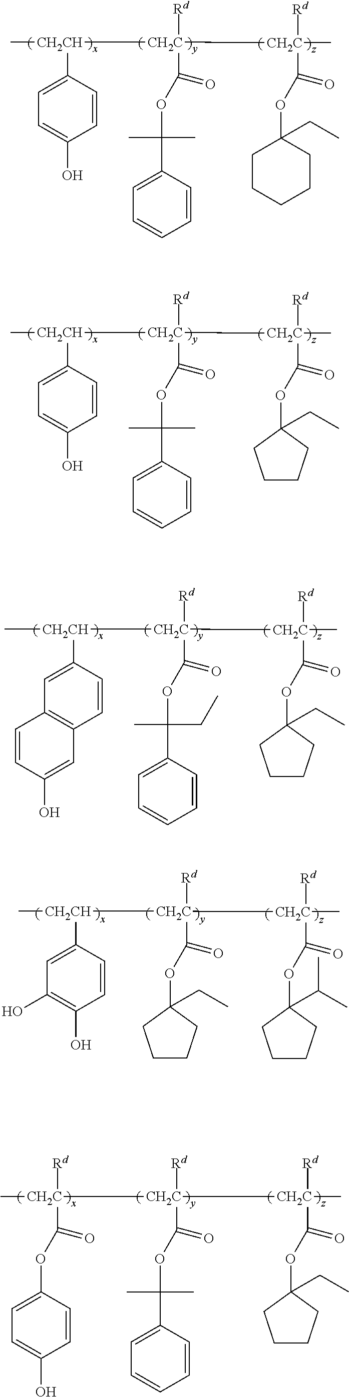

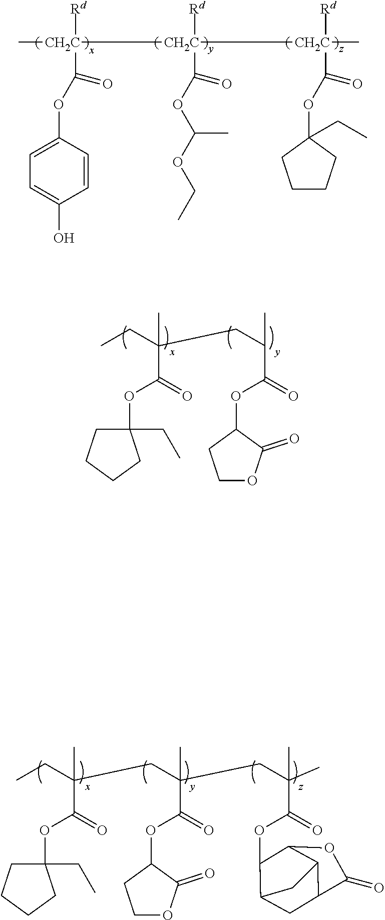

wherein each of x, y and z is a molar fraction of an associated repeating unit, wherein the sum of the molar fractions for each polymer adds up to 1, and wherein each R^{d} independently may be hydrogen, deuterium, halogen, cyano, substituted or unsubstituted C₁₋₁₀ alkyl, or substituted or unsubstituted C₃₋₆ cycloalkyl. Preferably, R^{d} may be hydrogen, fluorine, or substituted or unsubstituted C₁₋₅ alkyl, typically methyl.

The photoresist composition also includes a photoacid generator (PAG). The PAG may be in ionic or non-ionic form. The PAG may be in a polymeric or non-polymeric form. In polymeric form, the PAG may be present as a moiety in a repeating unit of a polymer that is derived from a polymerizable PAG monomer. In some embodiments, the PAG is not included in the structure of the first polymer.

In some embodiments, the photoresist composition may include two or more different photoacid generator compounds.

Suitable PAG compounds may be of the formula G⁺Z⁻, wherein G⁺ is an electroactive cation and Z⁻ is an anion that can generate a photoacid. The electroactive cation is preferably chosen from onium cations, preferably iodonium or sulfonium cations. Particularly suitable anions include those whose conjugated acids have a pKa of from −15 to 10. The anion is typically an organic anion having a sulfonate group or a non-sulfonate-type group, such as sulfonamidate, sulfonimidate, methide, or borate.

In some aspects, the anion of the PAG does not include and is free of —F, —CF₃, or —CF₂-groups. It should be understood that “free of —F, —CF₃, or —CF₂— groups” means that the anion of the PAG excludes groups such as —CH₂CF₃ and —CH₂CF₂CH₃. In still other aspects, the anion of the PAG is free of fluorine (i.e., does not contain a fluorine atom and is not substituted by a fluorine-containing group). In some aspects, the photoacid generator is free of fluorine (i.e., both the photoactive cation and the anion are free of fluorine).

The ionic PAG compound includes an organic cation, also referred to herein as G⁺. For example, the organic cation may be a sulfonium cation or an iodonium cation. In some embodiments, the organic cations may be a sulfonium cation of Formula (16) or an iodonium cation of Formula (17):

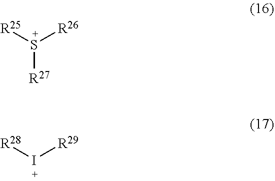

In Formulae (16) and (17), R²⁵ to R²⁹ are each independently substituted or unsubstituted C₁₋₂₀ alkyl, substituted or unsubstituted C₃₋₂₀ cycloalkyl, substituted or unsubstituted C₂₋₂₀ alkenyl, substituted or unsubstituted C₆₋₃₀ aryl, substituted or unsubstituted C₃₋₃₀ heteroaryl, substituted or unsubstituted C₇₋₂₀ arylalkyl, or substituted or unsubstituted C₄₋₂₀ heteroarylalkyl, or combinations thereof. Each of R²⁵ to R²⁷ may be either separate or connected to another group of R²⁵ to R²⁷ via a single bond or a divalent linking group to form a ring. R²⁸ and R²⁹ may be either separate or connected to each other via a single bond or a divalent linking group to form a ring. Each of R²⁵ to R²⁹ optionally may include as part of its structure a divalent linking group. Each of R²⁵ to R²⁹ independently may optionally comprise an acid-labile group chosen, for example, from tertiary alkyl ester groups, secondary or tertiary aryl ester groups, secondary or tertiary ester groups having a combination of alkyl and aryl groups, tertiary alkoxy groups, acetal groups, or ketal groups.

Exemplary sulfonium cations of Formula (16) include one or more of the following:

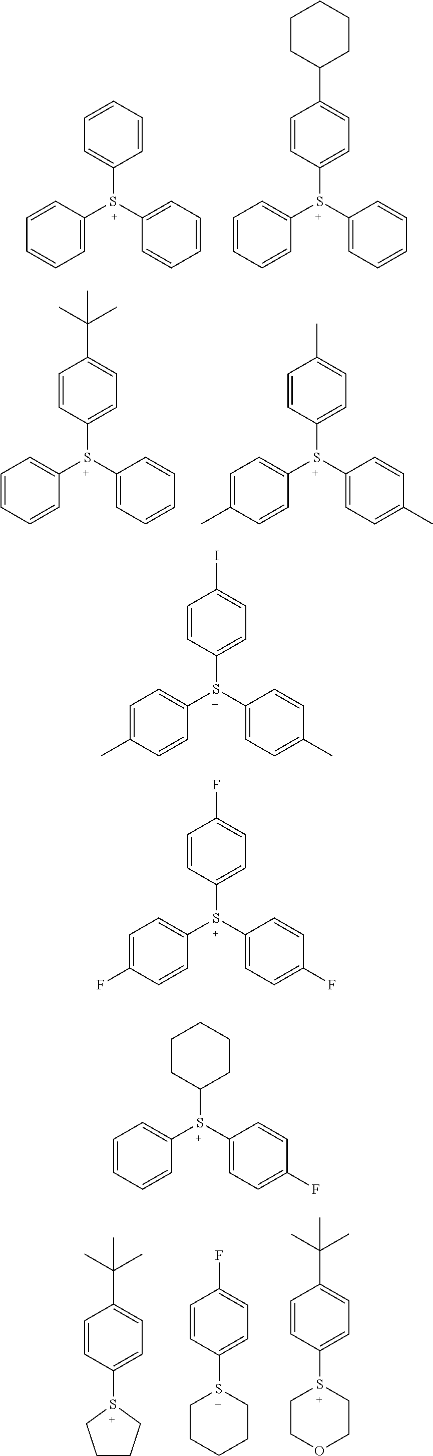

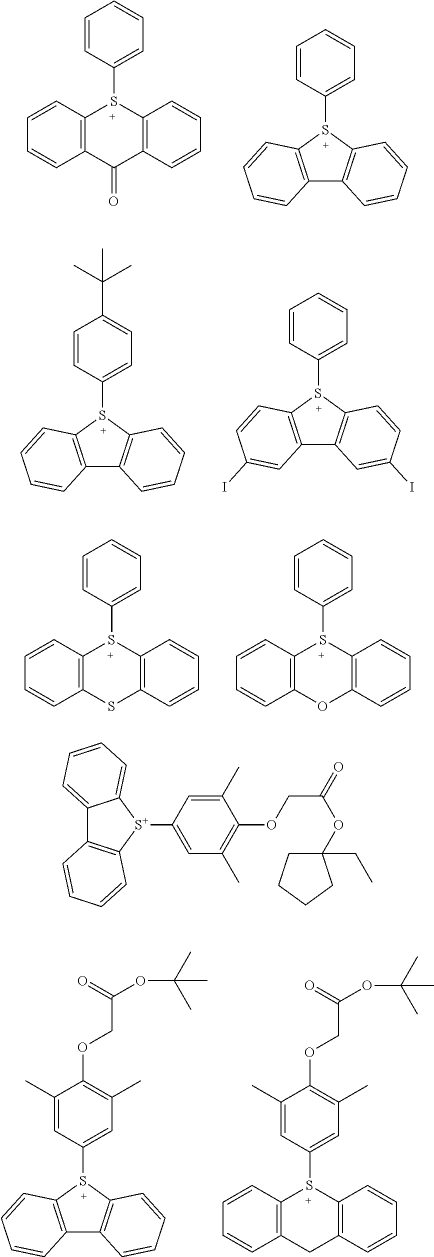

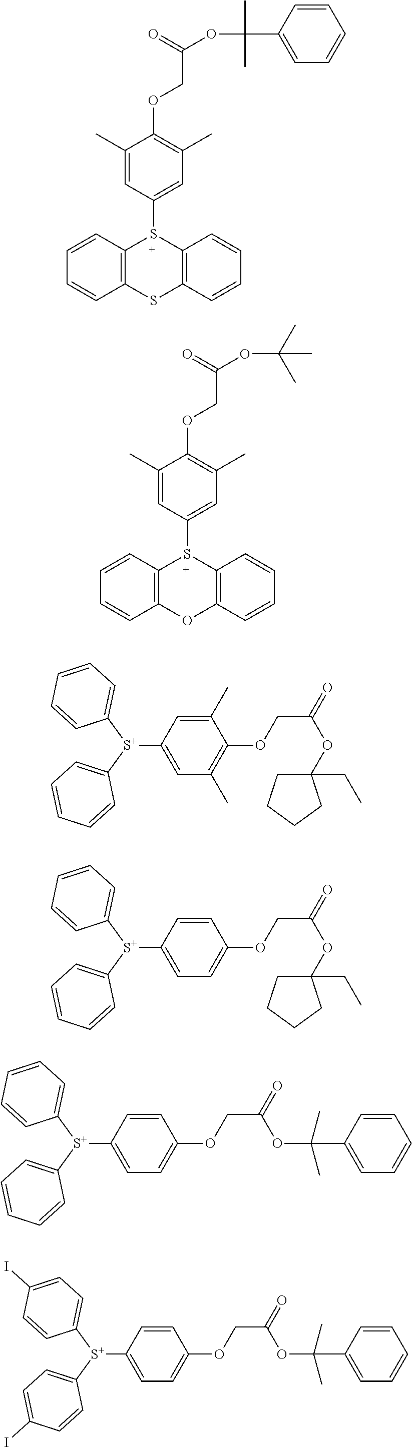

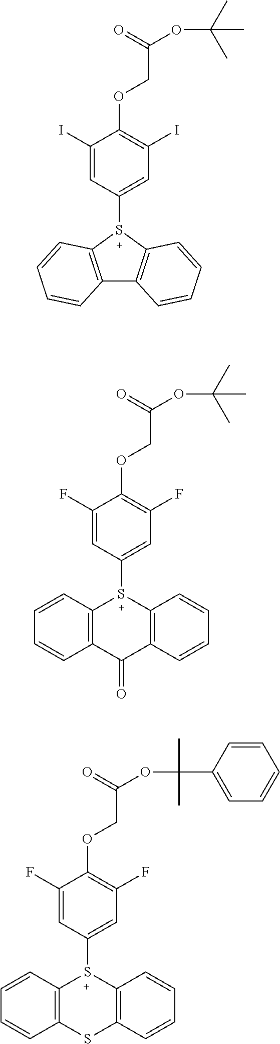

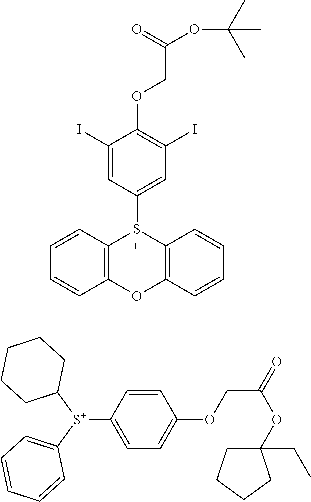

Exemplary iodonium cations of Formula (17) may include one or more of the following:

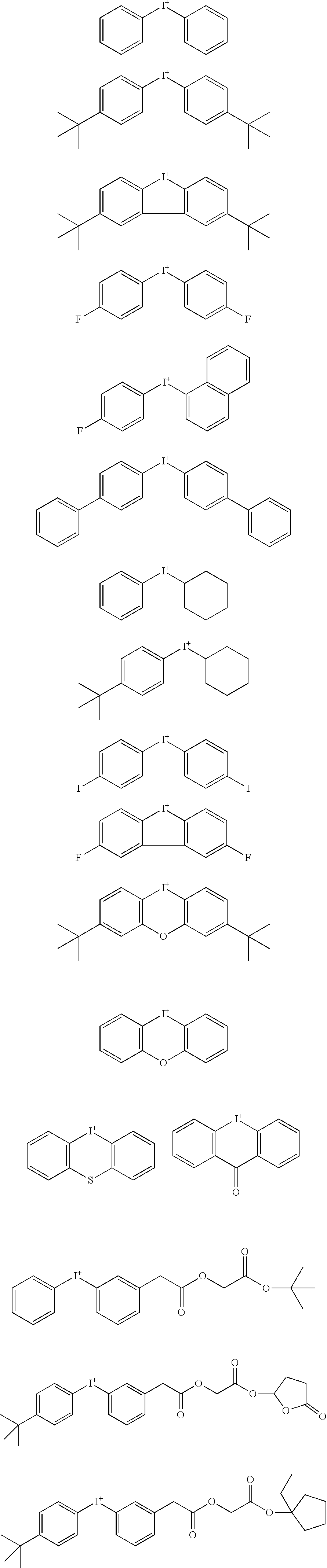

Exemplary organic anions, also referred to herein as Z⁻, having a sulfonate group include one or more of the following:

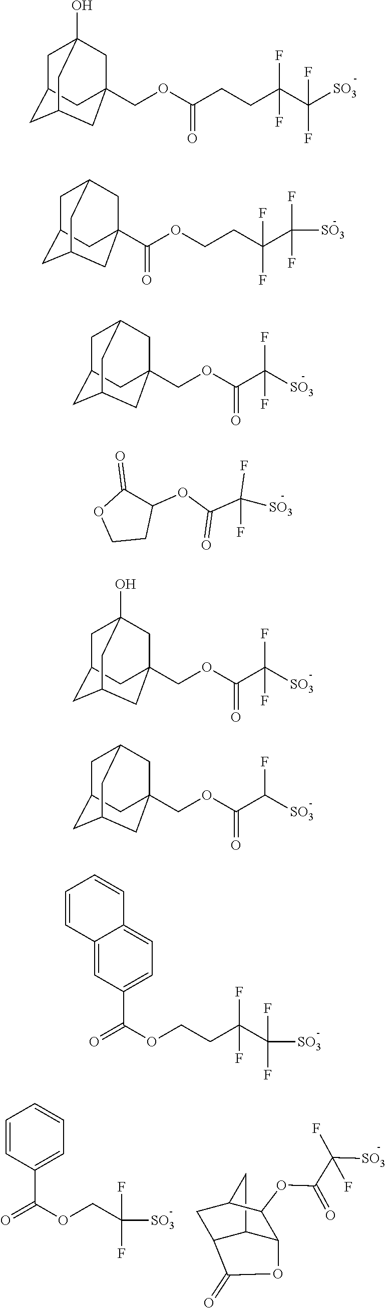

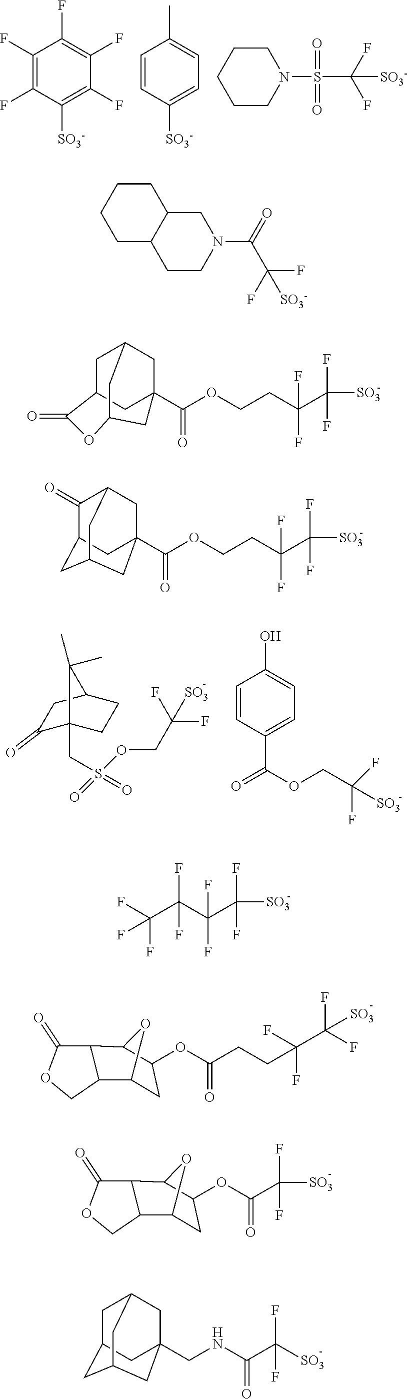

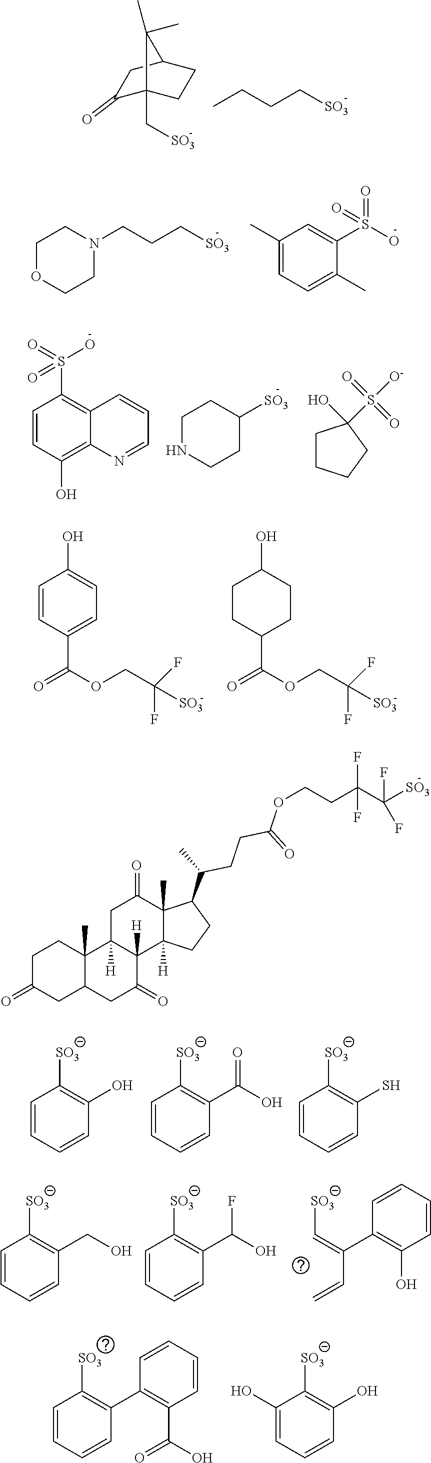

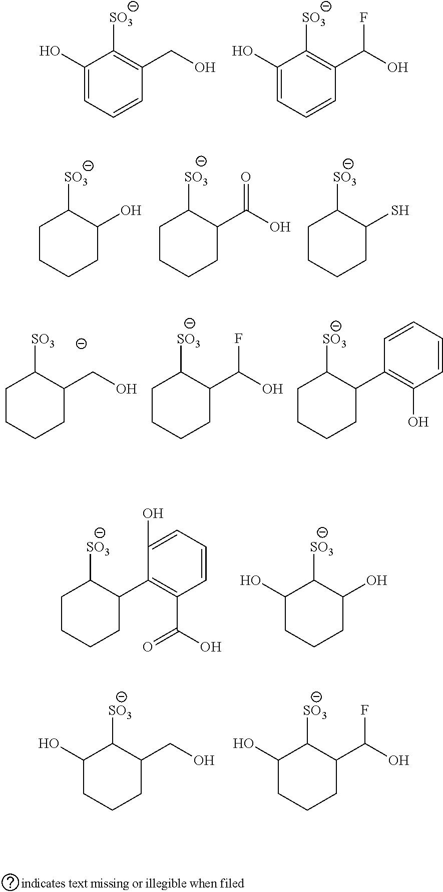

Exemplary non-sulfonated anions, also referred to herein as Z⁻, include one or more of the following:

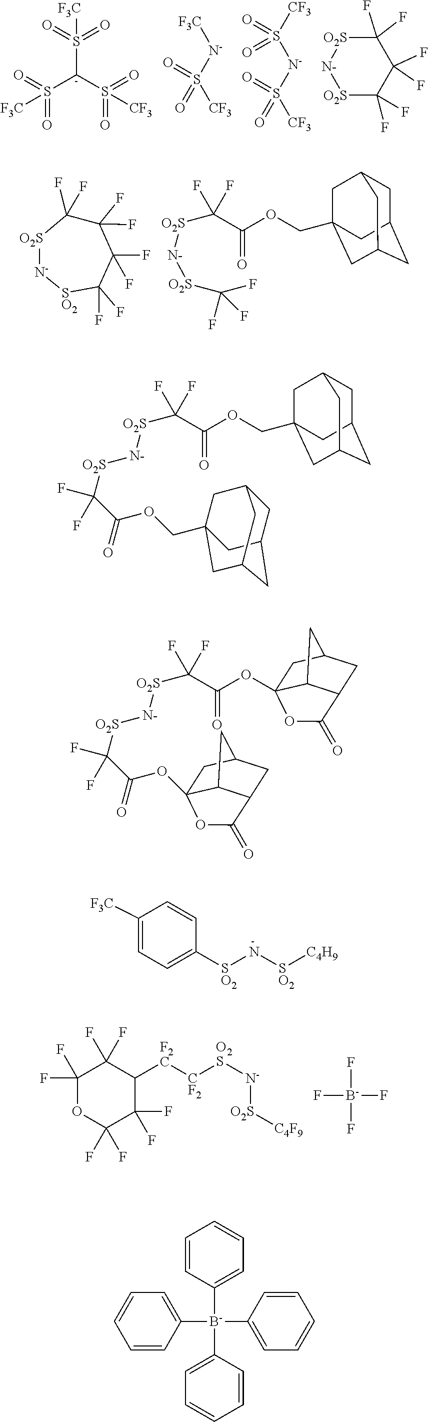

Commonly used onium salts may include, for example, triphenylsulfonium trifluoromethanesulfonate, (p-tert-butoxyphenyl)diphenylsulfonium trifluoromethanesulfonate, tris(p-tert-butoxyphenyl)sulfonium trifluoromethanesulfonate, triphenylsulfonium p-toluenesulfonate; di-t-butyphenyliodonium perfluorobutanesulfonate, and di-t-butyphenyliodonium camphorsulfonate. Other useful PAG compounds are known in the art of chemically amplified photoresists and include, for example: non-ionic sulfonyl compounds, for example, 2-nitrobenzyl-p-toluenesulfonate, 2,6-dinitrobenzyl-p-toluenesulfonate, and 2,4-dinitrobenzyl-p-toluenesulfonate; sulfonic acid esters, for example, 1,2,3-tris(methanesulfonyloxy)benzene, 1,2,3-tris(trifluoromethanesulfonyloxy)benzene, and 1,2,3-tris(p-toluenesulfonyloxy)benzene; diazomethane derivatives, for example, bis(benzenesulfonyl)diazomethane, bis(p-toluenesulfonyl)diazomethane; glyoxime derivatives, for example, bis-O-(p-toluenesulfonyl)-α-dimethylglyoxime, and bis-O-(n-butanesulfonyl)-α-dimethylglyoxime; sulfonic acid ester derivatives of an N-hydroxyimide compound, for example, N-hydroxysuccinimide methanesulfonic acid ester, N-hydroxysuccinimide trifluoromethanesulfonic acid ester; and halogen-containing triazine compounds, for example, 2-(4-methoxyphenyl)-4,6-bis(trichloromethyl)-1,3,5-triazine, and 2-(4-methoxynaphthyl)-4,6-bis(trichloromethyl)-1,3,5-triazine. Suitable photoacid generators are further described in U.S. Pat. Nos. 8,431,325 and 4,189,323.

In some embodiments, the PAG compound may be a non-ionic PAG compound. In some embodiments, the non-ionic PAG may include a sulfonyl-ester, an imino-ester, an imido-ester, or the like, or a combination thereof.

Exemplary non-ionic PAGs include one or more of the following:

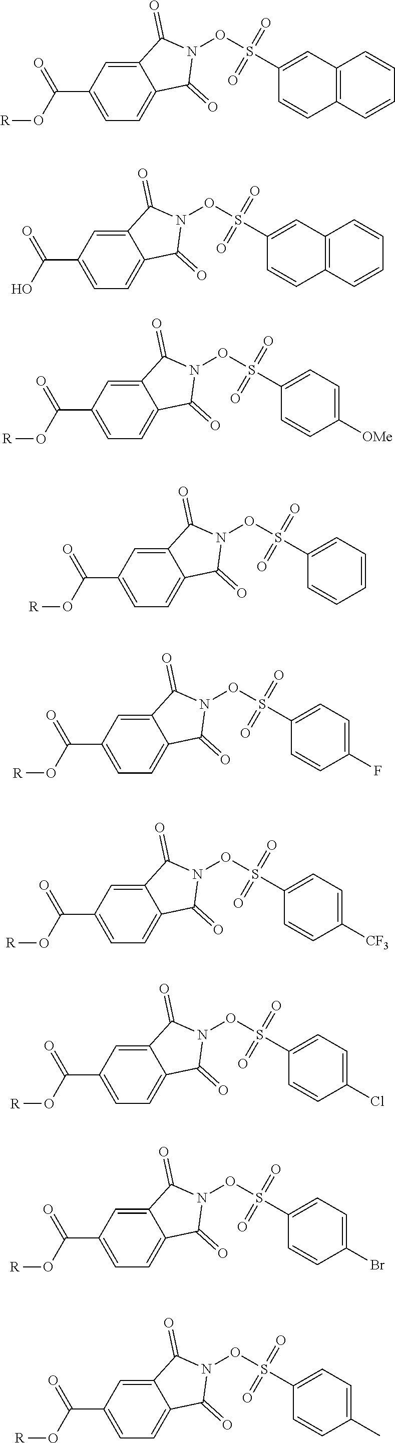

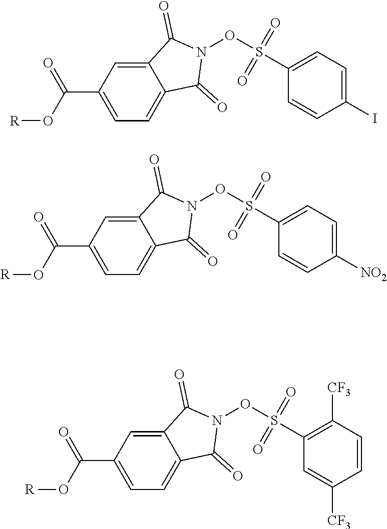

wherein R is a substituted or unsubstituted C₁₋₁₀ alkyl, or substituted or unsubstituted C₁₋₁₀ fluoroalkyl.

Typically, when the composition includes a non-polymeric PAG compound, the PAG compound is present in the composition in an amount of from 0.1 to 55 wt % o, more typically 1 to 25 wt %, based on total solids of the photoresist composition. When present in polymeric form, the PAG compound is typically included in a polymer in an amount from 1 to 25 mol %, more typically from 1 to 8 mol %, or from 2 to 6 mol %, based on total repeating units in the respective polymer.

The photoresist composition further includes a solvent for dissolving the components of the composition and to facilitate its coating on a substrate. Preferably, the solvent is an organic solvent conventionally used in the manufacture of electronic devices. Suitable solvents include, for example: aliphatic hydrocarbons such as hexane and heptane; aromatic hydrocarbons such as toluene and xylene; halogenated hydrocarbons such as dichloromethane (DCM), 1,2-dichloroethane and 1-chlorohexane; alcohols such as methanol, ethanol, 1-propanol, iso-propanol, tert-butanol, 2-methyl-2-butanol, 4-methyl-2-pentanol, and diacetone alcohol (4-hydroxy-4-methyl-2-pentanone) (DAA); propylene glycol monomethyl ether (PGME); ethers such as diethyl ether, tetrahydrofuran, 1,4-dioxane, and anisole; ketones such as acetone, methyl ethyl ketone, methyl iso-butyl ketone, 2-heptanone, and cyclohexanone (CHO); esters such as ethyl acetate, n-butyl acetate, propylene glycol monomethyl ether acetate (PGMEA), ethyl lactate (EL), hydroxyisobutyrate methyl ester (HBM), and ethyl acetoacetate; lactones such as gamma-butyrolactone (GBL) and epsilon-caprolactone; lactams such as N-methyl pyrrolidone; nitriles such as acetonitrile and propionitrile; cyclic or non-cyclic carbonate esters such as propylene carbonate, dimethyl carbonate, ethylene carbonate, and diphenyl carbonate; polar aprotic solvents such as dimethyl sulfoxide and dimethyl formamide; water; and combinations thereof. Of these, preferred solvents include one or more of PGME, PGMEA, EL, GBL, HBM, CHO, DAA, or a combination thereof.

The total solvent content (i.e., cumulative solvent content for all solvents) in the photoresist compositions is typically from 40 to 99 wt %, for example, from 60 to 99 wt %, or from 85 to 99 wt %, based on total solids of the composition. The desired solvent content will depend, for example, on the desired thickness of the coated (photoresist) layer and coating conditions.

In some embodiments, the photoresist composition in a coated dried state exhibits a receding contact angle of greater than 60° (before development). For example, the photoresist composition may have a receding contact angle of 60° to 90°, or 60° to 80° in a coated dried state and before development.

In some embodiments, the photoresist composition is free of fluorine.

Additionally, the compositions may further include one or more polymers in addition to and different from the polymer as described above. For example, the compositions may include an additional polymer as described above but different in composition. Additionally, or alternatively, the one or more additional polymers may include those well known in the photoresist art, for example, those chosen from polyacrylates, polyvinylethers, polyesters, polynorbornenes, polyacetals, polyethylene glycols, polyamides, polyacrylamides, polyphenols, novolacs, styrenic polymers, polyvinyl alcohols, or combinations thereof.

The composition may further include one or more additional, optional additives. For example, optional additives may include actinic and contrast dyes, anti-striation agents, plasticizers, speed enhancers, sensitizers, photo-decomposable quenchers (PDQ) (and, also known as photo-decomposable bases), basic quenchers, thermal acid generators, surfactants, and the like, or combinations thereof. If present, the optional additives are typically present in the compositions in an amount of from 0.01 to 20 wt %, based on total solids of the composition.

PDQs generate a relatively weak acid upon irradiation, such that the pKₐ of the acid generated by the PDQ is less than the pKₐ of the acid generated by the PAG. Exemplary photo-decomposable quenchers include, for example, photo-decomposable cations, and preferably those also useful for preparing strong acid generator compounds, paired with an anion of a weaker acid (pKₐ (PDQ)<pKₐ (PAG)) such as, for example, an anion of a C₁₋₂₀ carboxylic acid. Exemplary carboxylic acids include formic acid, acetic acid, propionic acid, tartaric acid, succinic acid, cyclohexanecarboxylic acid, benzoic acid, salicylic acid, and the like. In a preferred embodiment, the photo-decomposable quencher is a photo-decomposable organic zwitterion compound such as diphenyliodonium-2-carboxylate.

The PDQ may be in non-polymeric or polymer-bound form. The polymerized units containing the photo-decomposable quencher are typically present in an amount from 0.1 to 30 mol %, preferably from 1 to 10 mol % and more preferably from 1 to 2 mol %, based on total repeating units of the polymer. For example, the PDQ may be part of the second polymer, or may be part of an additional polymer. In some embodiments, the PDQ is not included in the first polymer.

Exemplary basic quenchers include, for example, linear aliphatic amines such as tributylamine, trioctylamine, triisopropanolamine, tetrakis(2-hydroxypropyl)ethylenediamine:n-tert-butyldiethanolamine, tris(2-acetoxy-ethyl) amine, 2,2′,2″,2′″-(ethane-1,2-diylbis(azanetriyl))tetraethanol, 2-(dibutylamino)ethanol, and 2,2′,2″-nitrilotriethanol; cyclic aliphatic amines such as 1-(tert-butoxycarbonyl)-4-hydroxypiperidine, tert-butyl 1-pyrrolidinecarboxylate, tert-butyl 2-ethyl-1H-imidazole-1-carboxylate, di-tert-butyl piperazine-1,4-dicarboxylate, and N-(2-acetoxy-ethyl)morpholine; aromatic amines such as pyridine, lutidine, di-tert-butyl pyridine, and pyridinium; linear and cyclic amides and derivatives thereof such as N,N-bis(2-hydroxyethyl)pivalamide, N,N-diethylacetamide, N¹,N¹,N³,N³-tetrabutylmalonamide, 1-methylazepan-2-one, 1-allylazepan-2-one, and tert-butyl 1,3-dihydroxy-2-(hydroxymethyl)propan-2-ylcarbamate; ammonium salts such as quaternary ammonium salts of sulfonates, sulfamates, carboxylates, and phosphonates; imines such as primary and secondary aldimines and ketimines; diazines such as optionally substituted pyrazine, piperazine, and phenazine; diazoles such as optionally substituted pyrazole, thiadiazole, and imidazole; and optionally substituted pyrrolidones such as 2-pyrrolidone and cyclohexyl pyrrolidine.

The basic quenchers may be in non-polymeric or polymer-bound form. When in polymeric form, the quencher may be present in repeating units of the polymer. The repeating units containing the quencher are typically present in an amount of from 0.1 to 30 mole %, preferably from 1 to 10 mole % and more preferably from 1 to 2 mole %, based on total repeating units of the polymer. For example, the basic quencher may be part of the second polymer, or may be part of an additional polymer. In some embodiments, the basic quencher is not included in the first polymer.

Exemplary surfactants include fluorinated and non-fluorinated surfactants and can be ionic or non-ionic, with non-ionic surfactants being preferable. Exemplary fluorinated non-ionic surfactants include perfluoro C₄ surfactants such as FC-4430 and FC-4432 surfactants, available from 3M Corporation; and fluorodiols such as POLYFOX PF-636, PF-6320, PF-656, and PF-6520 fluorosurfactants from Omnova. In an aspect, the composition further includes a surfactant polymer including a fluorine-containing repeating unit.

Another aspect provides a monomer including an ethylenically unsaturated double bond and a group of Formula (1A) or (2A) as defined herein, wherein * represents a point of bonding in the monomer.

Still another aspect provides a polymer including a repeating unit of Formula (1A) or (2A) as defined herein. For example, the polymer may include a repeating unit of Formula (1B) or (2B) as defined herein. Yet another aspect provides a composition that includes a polymer including a repeating unit of Formula (1A) or (2A), a photoacid generator, and a solvent.

Patterning methods using the photoresist compositions of the invention will now be described. Suitable substrates on which the photoresist compositions can be coated include electronic device substrates. A wide variety of electronic device substrates may be used in the present invention, such as: semiconductor wafers; polycrystalline silicon substrates; packaging substrates such as multichip modules; flat panel display substrates; substrates for light emitting diodes (LEDs) including organic light emitting diodes (OLEDs); and the like, with semiconductor wafers being typical. Such substrates are typically composed of one or more of silicon, polysilicon, silicon oxide, silicon nitride, silicon oxynitride, silicon germanium, gallium arsenide, aluminum, sapphire, tungsten, titanium, titanium-tungsten, nickel, copper, and gold. Suitable substrates may be in the form of wafers such as those used in the manufacture of integrated circuits, optical sensors, flat panel displays, integrated optical circuits, and LEDs. Such substrates may be any suitable size. Typical wafer substrate diameters are 200 to 300 millimeters (mm), although wafers having smaller and larger diameters may be suitably employed according to the present invention. The substrates may include one or more layers or structures which may optionally include active or operable portions of devices being formed.

Typically, one or more lithographic layers such as a hardmask layer, for example, a spin-on-carbon (SOC), amorphous carbon, or metal hardmask layer, a CVD layer such as a silicon nitride (SiN), a silicon oxide (SiO), or silicon oxynitride (SiON) layer, an organic or inorganic underlayer, or combinations thereof, are provided on an upper surface of the substrate prior to coating a photoresist composition of the present invention. Such layers, together with an overcoated photoresist layer, form a lithographic material stack.

Optionally, a layer of an adhesion promoter may be applied to the substrate surface prior to coating the photoresist compositions. If an adhesion promoter is desired, any suitable adhesion promoter for polymer films may be used, such as silanes, typically organosilanes such as trimethoxyvinylsilane, triethoxyvinylsilane, hexamethyldisilazane, or an aminosilane coupler such as gamma-aminopropyltriethoxysilane. Particularly suitable adhesion promoters include those sold under the AP 3000, AP 8000, and AP 9000S designations, available from Qnity Electronics, Inc. (Wilmington, Delaware).

The photoresist composition may be coated on the substrate by any suitable method, including spin coating, spray coating, dip coating, doctor blading, or the like. For example, applying the layer of photoresist may be accomplished by spin coating the photoresist in solvent using a coating track, in which the photoresist is dispensed on a spinning wafer. During dispensing, the wafer is typically spun at a speed of up to 4,000 rotations per minute (rpm), for example, from 200 to 3,000 rpm, for example, 1,000 to 2,500 rpm, for a period from 15 to 120 seconds to obtain a layer of the photoresist composition on the substrate. It will be appreciated by those skilled in the art that the thickness of the coated layer may be adjusted by changing the spin speed and/or the total solids of the composition. A photoresist composition layer formed from the compositions of the invention typically has a dried layer thickness from 3 to 30 micrometers (μm), preferably from greater than 5 to 30 μm, and more preferably from 6 to 25 μm.

The photoresist composition is typically next soft-baked to minimize the solvent content in the layer, thereby forming a tack-free coating and improving adhesion of the layer to the substrate. The soft bake is performed, for example, on a hotplate or in an oven, with a hotplate being typical. The soft bake temperature and time will depend, for example, on the photoresist composition and thickness. The soft bake temperature is typically from 80 to 170 °C, and more typically from 90 to 150 °C. The soft bake time is typically from 10 seconds to 20 minutes, more typically from 1 minute to 10 minutes, and still more typically from 1 minute to 2 minutes. The heating time can be readily determined by one of ordinary skill in the art based on the ingredients of the composition.

The photoresist layer is next pattern-wise exposed to activating radiation to create a difference in solubility between exposed and unexposed regions. Reference herein to exposing a photoresist composition to radiation that is activating for the composition indicates that the radiation can form a latent image in the photoresist composition. The exposure is typically conducted through a patterned photomask that has optically transparent and optically opaque regions corresponding to regions of the resist layer to be exposed and unexposed, respectively. Such exposure may, alternatively, be conducted without a photomask in a direct writing method, typically used for e-beam lithography. The activating radiation typically has a wavelength of sub-400 nm, sub-300 nm or sub-200 nm, with 248 nm (KrF), 193 nm (ArF), 13.5 nm (EUV) wavelengths or e-beam lithography being preferred. Preferably, the activating radiation is 248 nm radiation. The methods find use in immersion or dry (non-immersion) lithography techniques. The exposure energy is typically from 1 to 200 millijoules per square centimeter (mJ/cm²), preferably from 10 to 100 mJ/cm² and more preferably from 20 to 50 mJ/cm², dependent upon the exposure tool and components of the photoresist composition.

Following exposure of the photoresist layer, a post-exposure bake (PEB) of the exposed photoresist layer is performed. The PEB can be conducted, for example, on a hotplate or in an oven, with a hotplate being typical. Conditions for the PEB will depend, for example, on the photoresist composition and layer thickness. The PEB is typically conducted at a temperature from 70 to 150 °C, preferably from 75 to 120 °C, and a time from 30 to 120 seconds. A latent image defined by the polarity-switched (exposed regions) and unswitched regions (unexposed regions) is formed in the photoresist.

The exposed photoresist layer is then developed with a suitable developer to selectively remove those regions of the layer that are soluble in the developer while the remaining insoluble regions form the resulting photoresist pattern relief image. In the case of a positive-tone development (PTD) process, the exposed regions of the photoresist layer are removed during development and unexposed regions remain. Conversely, in a negative-tone development (NTD) process, the exposed regions of the photoresist layer remain, and unexposed regions are removed during development. Application of the developer may be accomplished by any suitable method such as described above with respect to application of the photoresist composition, with spin coating being typical. The development time is for a period effective to remove the soluble regions of the photoresist, with a time of from 5 to 60 seconds being typical. Development is typically conducted at room temperature.

Suitable developers for a PTD process include aqueous base developers, for example, quaternary ammonium hydroxide solutions such as tetramethylammonium hydroxide (TMAH), preferably 0.26 normal (N) TMAH, tetraethylammonium hydroxide, tetrabutylammonium hydroxide, sodium hydroxide, potassium hydroxide, sodium carbonate, potassium carbonate, and the like. Suitable developers for an NTD process are organic solvent-based, meaning the cumulative content of organic solvents in the developer is 50 wt % or more, typically 95 wt % or more, 98 wt % or more, or 100 wt %, based on total weight of the developer. Suitable organic solvents for the NTD developer include, for example, those chosen from ketones, esters, ethers, hydrocarbons, and mixtures thereof. The developer is typically 2-heptanone or n-butyl acetate.

A coated substrate may be formed from the photoresist compositions of the invention. Such a coated substrate includes: (a) a substrate having one or more layers to be patterned on a surface thereof; and (b) a layer of the photoresist composition over the one or more layers to be patterned.

The photoresist pattern may be used, for example, as an etch mask, thereby allowing the pattern to be transferred to one or more sequentially underlying layers by known etching techniques, typically by dry-etching such as reactive ion etching. The photoresist pattern may, for example, be used for pattern transfer to an underlying hardmask layer which, in turn, is used as an etch mask for pattern transfer to one or more layers below the hardmask layer. If the photoresist pattern is not consumed during pattern transfer, it may be removed from the substrate by known techniques, for example, oxygen plasma ashing. The photoresist compositions may, when used in one or more such patterning processes, be used to fabricate semiconductor devices such as memory devices, processor chips (CPUs), graphics chips, optoelectronic chips, LEDs, OLEDs, as well as other electronic devices.

The subject matter is further illustrated by the following non-limiting examples.

### Examples

### List of Monomers in Synthesis Examples

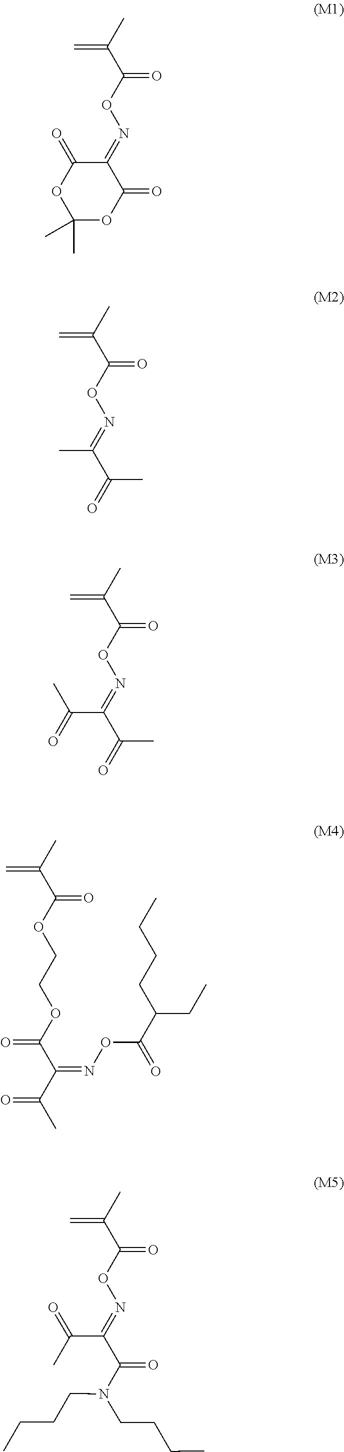

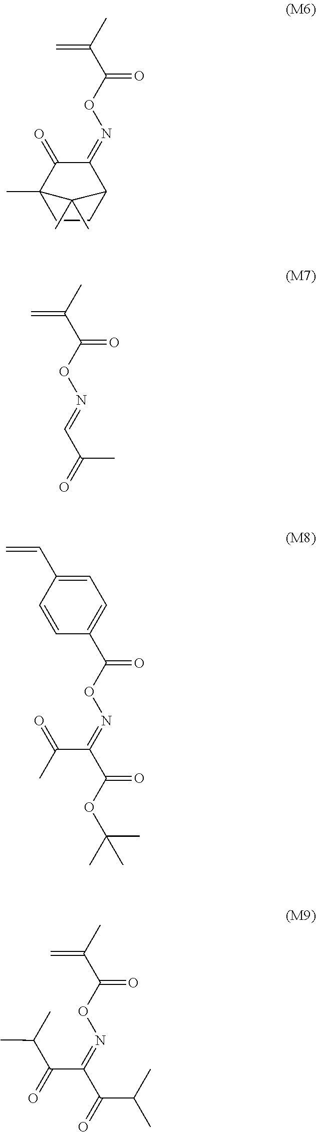 [SMILES: C=C(C)C(=O)O/N=C1\C(=O)C2(C)CCC1C2(C)C ; C=C(C)C(=O)O/N=C/C(C)=O ; C=Cc1ccc(C(=O)O/N=C(\C(C)=O)C(=O)OC(C)(C)C)cc1 ; C=C(C)C(=O)ON=C(C(=O)C(C)C)C(=O)C(C)C]

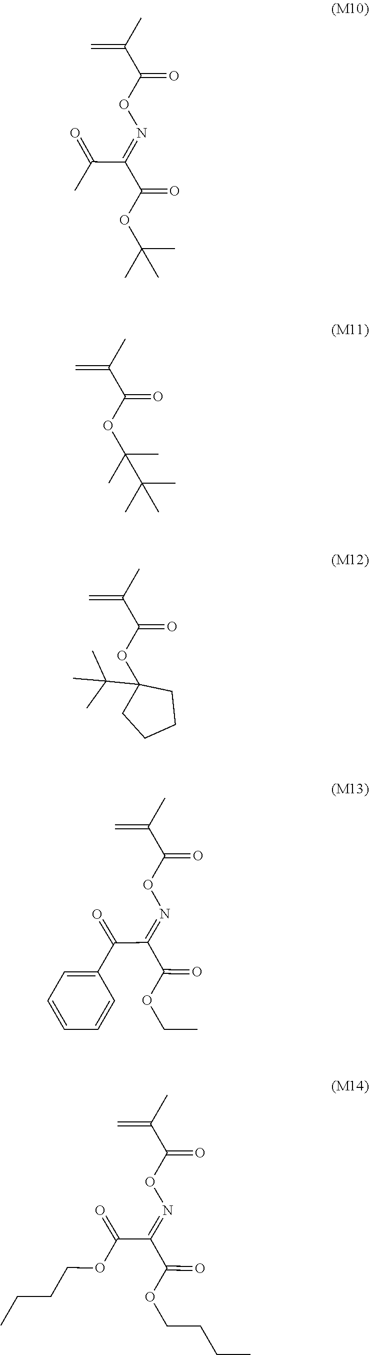 [SMILES: C=C(C)C(=O)O/N=C(\C(C)=O)C(=O)OC(C)(C)C ; C=C(C)C(=O)OC(C)(C)C(C)(C)C ; C=C(C)C(=O)OC1(C(C)(C)C)CCCC1 ; C=C(C)C(=O)O/N=C(/C(=O)OCC)C(=O)c1ccccc1 ; C=C(C)C(=O)ON=C(C(=O)OCCCC)C(=O)OCCCC]

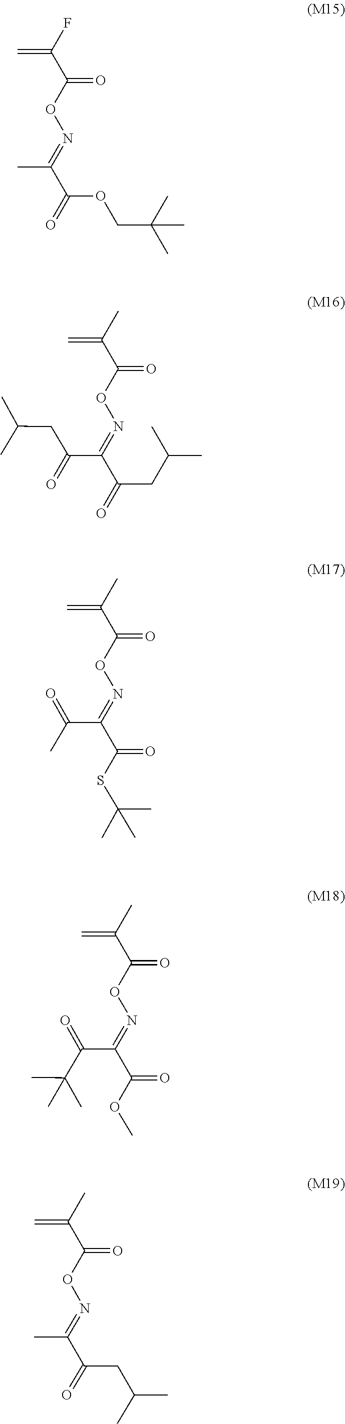

### Synthesis of 2,2-dimethyl-1,3-dioxane-4,5,6-trione 5-(O-methacryloyloxime) (M1)

The monomer designated M1 was prepared as shown below.

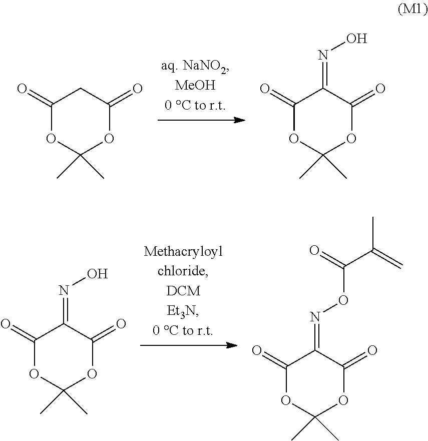

Under a nitrogen atmosphere, to a solution of 30.0 g (208 mmol) of Meldrum's acid in 210 mL methanol (MeOH) cooled at 0 °C was added a solution of 17.25 g (250 mmol, 1.2 eq.) of sodium nitrite in 40 mL of water dropwise over a 14 minute period with stirring. The reaction was allowed to warm to room temperature slowly while stirring. After stirring for an additional 4.5 hours at room temperature, the reaction was concentrated under reduced pressure. Next, 150 mL of 10% aqueous (aq.) HCl and ~200 mL of dichloromethane (DCM) were added thereto. After stirring at room temperature for 6 minutes, 10 mL of 12.1 M aqueous HCl was added thereto. The contents were extracted with dichloromethane, and the combined extracts were dried with sodium sulfate, filtered, and concentrated under reduced pressure to give 28.7 g (79.4%) of 2,2-dimethyl-1,3-dioxane-4,5,6-trione-5-oxime as a slightly tan solid.

To a solution of 11.7 g (67.6 mmol) of 2,2-dimethyl-1,3-dioxane-4,5,6-trione-5-oxime in 350 mL of anhydrous dichloromethane cooled at 0 °C was added 9.4 mL of triethylamine (67.6 mmol, Et₃N), and then 6.6 mL (67.6 mmol) of methacryloyl chloride in 36 mL dichloromethane was added thereto over a 3.5 minute period with stirring. Upon completion, the reaction was concentrated under reduced pressure to yield an orange solid. Ethyl acetate was added to dissolve the crude product, which was then filtered and concentrated under a reduced pressure. The crude product was dissolved in dichloromethane and purified via silica gel chromatography in two portions on a Teledyne ISCO CombiFlash system using a 330 g RediSep Silver silica gel column eluting with dichloromethane at 150 mL/minute to produce 4.98 g (30.6%) of the product M1 as an off-white solid (7.3 mg of 4-methoxyphenol (MEHQ) was added for stability).

### Synthesis of 2,3-butanedione-O-(methacryloyl)oxime (M2)

The synthetic scheme for the monomer designated M2 is shown below.

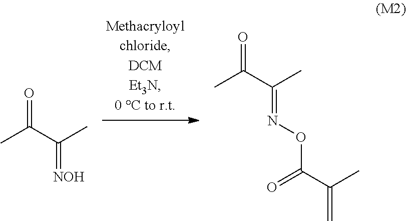

Under a nitrogen atmosphere, a solution of 25.0 g (247 millimoles (mmol)) of 2,3-butanedione monoxime in 500 mL of dichloromethane and 38 mL (272 mmol, 1.1 eq.) of triethylamine was prepared. The solution was cooled to 0 °C. Then, 26.5 mL (272 mmol, 1.1 eq.) of methacryloyl chloride was added dropwise over a 12.5 minute period with stirring. The reaction was then brought to room temperature. After 19 hours of stirring at room temperature, the reaction was filtered, and the filtrate was concentrated under reduced pressure. Ethyl acetate was added to the solid and the mixture was filtered. The filtrate was concentrated under reduced pressure. The crude material was dissolved in dichloromethane and purified via silica gel column chromatography on a Teledyne ISCO CombiFlash system using 330 g RediSep Silver silica gel columns eluted with dichloromethane to yield 14.9 g (35.5%) of the product M2 as a slightly pale yellow colored solid.

### Synthesis of 2,4-pentanedione-O-methacryloyloxime (M3)

The synthetic scheme for the monomer designated M3 is shown below.

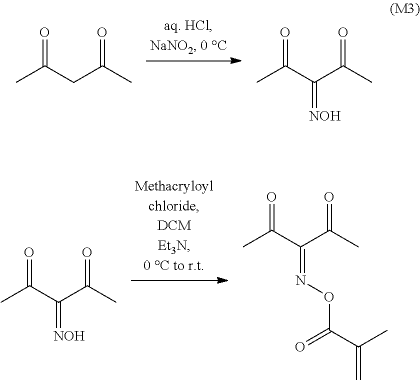

Under a nitrogen atmosphere, to a solution of 30.0 g (60 mmol) of acetylacetone was added 158.6 g of ice. Then, 36 mL (87 mmol, 1.45 eq.) of concentrated aqueous HCl was added, and the mixture was cooled to 0 °C. A solution of 21.0 g (60.3 mmol, 1.0 eq.) of sodium nitrite in 45 mL of water was added over 13 minutes with stirring. The reaction was then brought to room temperature with continued stirring. After 1 hour, 50 mL of water was added to ensure efficient stirring. After an additional 2.5 hours of stirring, brine was added to the reaction. The contents were extracted with ethyl acetate, and the combined extracts were dried with sodium sulfate, filtered, and concentrated under reduced pressure to give 34.97 g (90%) of 3-(hydroxyimino)pentane-2,4-dione as a white solid.

Under a nitrogen atmosphere, a solution of 18.677 g (145 mmol) of 3-(hydroxyimino)pentane-2,4-dione in 200 mL of dichloromethane and 20.16 mL (145 mmol, 1.0 eq.) of triethylamine was prepared. The solution was cooled to 0 °C. Then, a solution of 14.13 mL (145 mmol, 1.0 eq.) of methacryloyl chloride in 60 mL of dichloromethane was added over a 37 minute period with stirring. The reaction was allowed to warm to room temperature. After 10.5 hours of stirring at room temperature, the reaction was filtered, and the filtrate was concentrated under reduced pressure. Ethyl acetate (300 mL) was added thereto, and the mixture was filtered. The filtrate was concentrated under reduced pressure. The crude material was dissolved in 3:1 dichloromethane:hexanes and purified on a Teledyne ISCO CombiFlash system using 330 g RediSep Silver silica gel columns eluting with 2:1 dichloromethane:hexanes at 165 mL/minute for 13.5 minutes, and then using 5:1 dichloromethane:hexanes. The product M3 crystallized upon standing and was refrigerated for 10 minutes to ensure complete crystallization. Yield: 16.85 g as a white solid (64.8 mg of MEHQ was added to preserve stability).

### Synthesis of 2-methacryloyloxyethyl-2-[O-(2-ethylhexanoyl)oxime]-3-oxobutanoate (M4)

The monomer designated M4 was prepared as shown below.

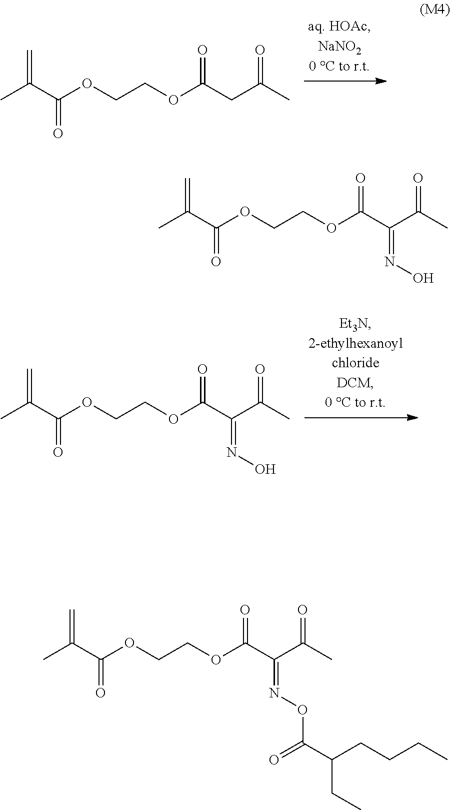

To a solution of 20.0 g (93.4 mmol) of 2-(methacryloyloxy)ethyl acetate in 50 mL of glacial acetic acid (HOAc) at 0 °C was added a solution of 6.44 g (93.4 mmol, 1.0 eq.) of sodium nitrite in 50 mL of water dropwise over 3 minutes under a nitrogen atmosphere and with stirring. The reaction was allowed to warm to room temperature slowly overnight with stirring, whereupon water was added to the reaction and the contents were extracted with dichloromethane. The combined dichloromethane layers were washed once with saturated aqueous sodium bicarbonate, and the dichloromethane extracts were dried with sodium sulfate, filtered, and the filtrate was concentrated under reduced pressure to give a deep gold-colored, moderately viscous liquid. The crude product was purified on a 326 g, 3-inch diameter silica gel column eluting with 95:5 dichloromethane:ethyl acetate to give 9.5 g of (2-methacryloyloxy)-2-(hydroxyimino)-3-oxobutanoate as an oil (17.2 mg of MEHQ was added to preserve stability).

Under an atmosphere of nitrogen, to a stirred solution of 9.5 g (39.1 mmol) of (2-methacryloyloxy)-2-(hydroxyimino)-3-oxobutanoate in 200 mL of dichloromethane at 0 °C were added 40 mg (0.31 mmol, 0.008 eq.) of MEHQ, 5.5 mL (40.2 mmol, 1.03 eq.) of triethylamine, and 6.8 mL (39.7 mmol, 1.02 eq.) of 2-ethylhexanoyl chloride in 14 mL of dichloromethane over three minutes. After 25 minutes of stirring at 0 °C, the reaction was then stirred at room temperature. After 3 hours of stirring at room temperature, the reaction was concentrated under reduced pressure to a 20 mL volume. Ethyl acetate was added, and the mixture was filtered. The filtrate was concentrated under reduced pressure to give a slightly viscous orange/gold colored liquid. The crude material was dissolved in 5:1 dichloromethane:hexanes and purified on a Teledyne ISCO CombiFlash system using a 330 g RediSep Silver silica gel column eluting with 5:1 dichloromethane:hexanes at 170 mL/minute for 8.8 minutes then at 190 mL/minute for 2.2 minutes, then with dichloromethane at 190 mL/minute. The product M4 was obtained as a colorless oil (19.6 mg of MEHQ was added to preserve stability). Yield: 7.65 g (53%).

### Synthesis of N,N-dibutyl-2-((methacryloyloxy)imino)-3-oxobutanamide (M5)

In a round bottom flask, 14.6 g of dibutyl amine, 8.4 g of 2,2,6-trimethyl-4H-1,3-dioxin-4-one, and 50 mL of toluene were combined. The mixture was stirred and heated at reflux for 16 hours, before being cooled to room temperature and washed with a dilute aqueous HCl solution. The organic layer was collected and dried to yield N,N-dibutyl-3-oxobutanamide.

At about 10 °C, the amide was dissolved in 50 mL of acetic acid; and then 5.0 g of NaNO₂ in 12 mL of water was slowly added over the course of 30 minutes. After reacting for another hour at room temperature, the product was isolated by extraction with methyl t-butyl ether from water and washing with aqueous NaHCO₃ until the aqueous layer became neutral. 18.3 g of a slightly brownish solid oxime precursor was obtained.

At 0 °C, the oxime precursor was dissolved in 100 mL of dichloromethane with 5.5 g of methacrylic acid and 50 mg of 4-pyrrolidinopyridine. Then, 12.0 g of N-(3-dimethylaminopropyl)-N′-ethylcarbodiimide hydrochloride (EDC) was slowly added over 1 hour. After reacting at room temperature for another hour with stirring, the product was isolated by extraction with methyl t-butyl ether from water and washing with aqueous HCl and aqueous NaHCO₃ solutions. A brownish honey-like product was obtained after concentration (with 30 mg of BHT) under reduced pressure. The material was redissolved in 30 mL methanol, and dropped into 70 g of ice-water mixture. The solid was collected and dried to yield 6.1 g of reddish yellow solid as monomer M5.

### Synthesis of 1,7,7-trimethylbicyclo[2.2.1]heptane-2,3-dione-3-(O-methacryloxime) (M6)

The monomer designated M6 was prepared as shown below.

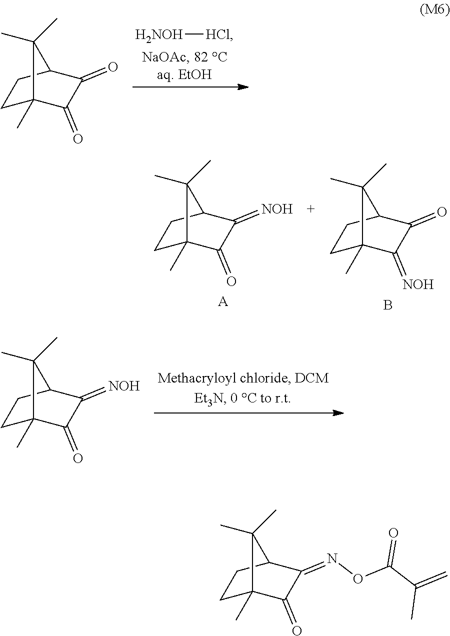

To a solution of 20 g (120 mmol) of (+)-camphorquinone in 100 mL of ethanol was added a solution of 9.87 g (120 mmol, 1.0 eq.) of sodium acetate in 21.5 mL of water. Next, a solution of 8.36 g (31.2 mmol, 1.0 eq.) of hydroxylamine hydrochloride in 23.4 mL of water at room temperature was added thereto. The reaction was heated at 82 °C for 2.25 hours and then stirred at room temperature. After stirring for 17 additional hours at room temperature, the reaction was filtered and then concentrated under reduced pressure to remove the ethanol. The contents were extracted with ethyl acetate, and the combined extracts were dried with magnesium sulfate, filtered, and concentrated under reduced pressure. The crude material, a combination of 1,7,7-trimethylbicyclo[2.2.1]heptane-2,3-dione 2-oxime (B) and 1,7,7-trimethylbicyclo[2.2.1]heptane-2,3-dione 3-oxime (A), was purified on a Teledyne ISCO CombiFlash system using a 330 g RediSep Silver silica gel column, eluting with 95:5 dichloromethane:ethyl acetate at 150 mL/minute. The second major peak was collected to give 15.0 g (69%) of 1,7,7-trimethylbicyclo[2.2.1]heptane-2,3-dione 3-oxime as a white solid.

Under a nitrogen atmosphere, to a solution of 9.16 g (50.5 mmol) of 1,7,7-trimethylbicyclo[2.2.1]heptane-2,3-dione 3-oxime in 150 mL dichloromethane and 7.1 mL (50.5 mmol, 1.0 eq.) of triethylamine cooled at 0 °C was added 5.0 mL (50.5 mmol, 1.0 eq.) of methacryloyl chloride in 50 mL of dichloromethane over a 23 minute period with stirring. The reaction was allowed to warm slowly to room temperature. After 18 hours of stirring at room temperature, the reaction was concentrated under reduced pressure. Ethyl acetate was added, and the resulting mixture was filtered. The filtrate was concentrated under reduced pressure. The crude material was purified on a Teledyne ISCO CombiFlash system using a 330 g RediSep Silver silica gel column eluting with a gradient of 100% dichloromethane to 95:5 dichloromethane:ethyl acetate over 40 minutes. The product M6 was obtained as a yellow colored solid (6.6 mg of MEHQ was added to preserve stability). Yield: 4.7 g (37%).

### Synthesis of 2-oxopropanal-O-methacryloyloxime (M7)

The synthetic scheme for the monomer designated M7 is shown below.

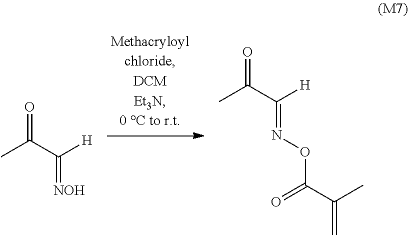

A solution of 40 grams (g) of isonitrosoacetone in 60 milliliters (mL) of dichloromethane was purified via silica gel column chromatography using 252 g of silica gel and eluted with dichloromethane:ethyl acetate (95:5) to provide 31.7 g of isonitrosoacetone as an off-white solid.

To a solution of the purified isonitrosoacetone in 500 mL of dichloromethane under a nitrogen atmosphere at 0 °C was added 35.0 mL of triethylamine. Next, a solution of 24.6 mL of methacryloyl chloride in 25 mL of dichloromethane was added dropwise over a 30 minute period with stirring. The reaction was allowed to warm slowly to room temperature with stirring. After completion, the reaction was concentrated under reduced pressure. Ethyl acetate was added to the crude material and the mixture was filtered. The filtrate was concentrated under reduced pressure. The crude material was dissolved in dichloromethane and purified via silica gel column chromatography using a Teledyne ISCO CombiFlash system using 330 g RediSep Silver silica gel columns eluted with dichloromethane at 150 mL/minute to produce 3.60 g (10%) of the product M7 as a slightly gold colored liquid.

### Synthesis of tert-butyl (E)-3-oxo-2-(((4-vinylbenzoyl)oxy)imino)butanoate (M8)

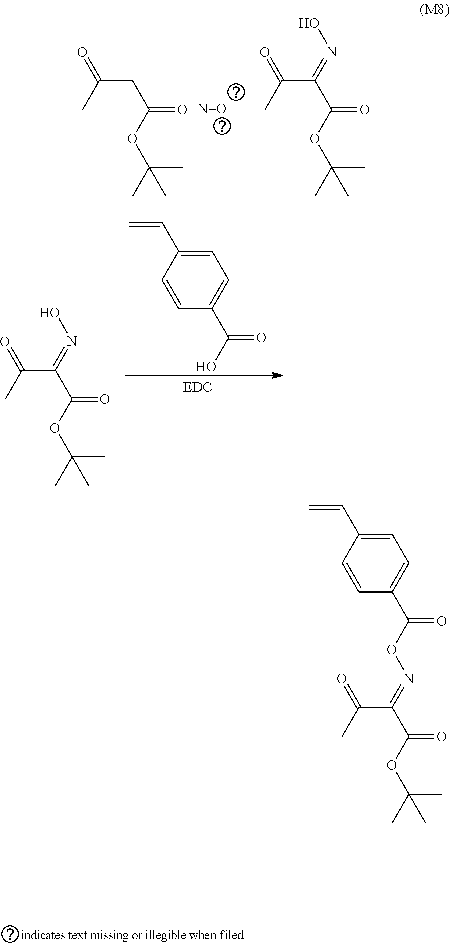

The monomer designated M8 was prepared as follows. At 0 °C, 15.3 mL of t-butyl acetoacetate was dissolved in 100 mL of acetic acid; and then 7.5 g of NaNO₂ (to generate O═N⁺ in-situ) in 25 mL of water was slowly added over the course of 30 minutes; after reacting for another hour at room temperature, the product was isolated by extraction with methyl t-butyl ether from water and washing with aqueous NaHCO₃. 17.2 g of slightly yellow solid precursor was obtained. At 0 °C, 7.9 g of the solid precursor was dissolved in 100 mL of dichloromethane with 7.5 g of vinylbenzoic acid; then, 9.1 g of EDC was slowly added over 30 minutes; after reacting at room temperature for another hour, the product was isolated by extraction with methyl t-butyl ether from water and washing with HCl and NaHCO₃. A solid product was obtained after concentration (with 0.1 g of BHT). The solid was redissolved in 70 mL of warm heptane (~60 °C), the solution was filtered through a 10-micron PE filter with a layer of Na₂SO₄, and cooled to −78 °C, and the solid was collected. The recrystallization process was repeated again to yield 5.8 g of a slightly yellow crystalline solid product M8.

### Synthesis of 2,6-dimethylheptane-3,4,5-trione-4-(O-methacryoyloxime) (M9)

The monomer designated M9 was prepared as shown below.

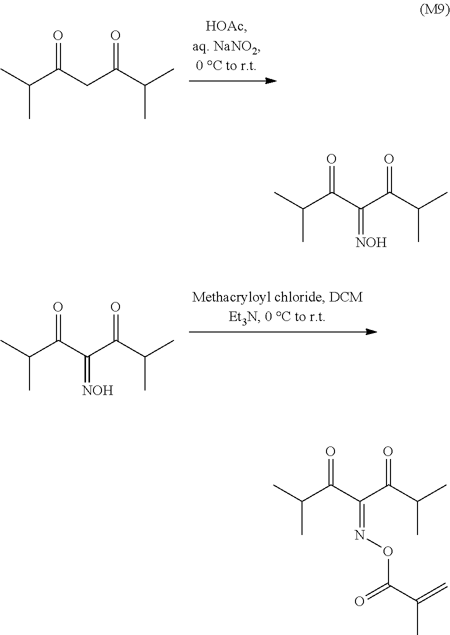

Under a nitrogen atmosphere, a solution of 50.0 g (0.32 mol) of 2,6-dimethyl-3,5-heptanedione in 200 mL of glacial acetic acid was cooled to 0 °C. A solution of 88.3 g (1.28 mol, 4.0 eq.) of sodium nitrite in 130 mL of water was added portion-wise over ~13.5 minutes with stirring, maintaining a temperature between 0 °C and room temperature. After the addition was completed, the reaction was allowed to warm slowly to room temperature with stirring. After 16 hours of stirring at room temperature, brine was added to the reaction and the contents were extracted with ethyl acetate. The combined extracts were dried with sodium sulfate, filtered, and concentrated under reduced pressure to provide a gold-orange colored viscous liquid. The crude material was purified via silica gel column chromatography using 636 g of silica gel and eluted with dichloromethane. A slightly gold-colored oil of 4-(hydroxyimino)-2,6-dimethylheptane-3,5-dione was obtained. Yield: 6.1 g.

Under a nitrogen atmosphere, to a solution of 5.78 g (31.2 mmol) of 4-(hydroxyimino)-2,6-dimethylheptane-3,5-dione in 50 mL of dichloromethane at 0 °C was added 4.4 mL (31.2 mmol, 1.0 eq.) of triethylamine, and then 3.1 mL (31.2 mmol, 1.0 eq.) of methacryloyl chloride in 22 mL of dichloromethane was added over a 13 minute period with stirring. The reaction was allowed to warm to room temperature. An additional 28 mL of dichloromethane was added to ensure vigorous stirring. After 2.5 hours of stirring at room temperature, the reaction was concentrated under reduced pressure. Ethyl acetate (100 mL) was added and the mixture was filtered. The filtrate was concentrated under reduced pressure to give an orange/gold colored liquid. The crude material was dissolved in 1:1 dichloromethane:hexanes and purified on a Teledyne ISCO CombiFlash system using a 330 g RediSep Silver silica gel column eluting with 2:1 dichloromethane:hexanes at 165 mL/minute. The product M9 was obtained as a very light yellow colored liquid (4.2 mg of MEHQ was added to preserve stability). Yield: 6.1 g (51%).

### Synthesis of tert-butyl (E)-2-((methacryloyloxy)imino)-3-oxobutanoate (M10)

The monomer designated M10 was prepared as shown below.

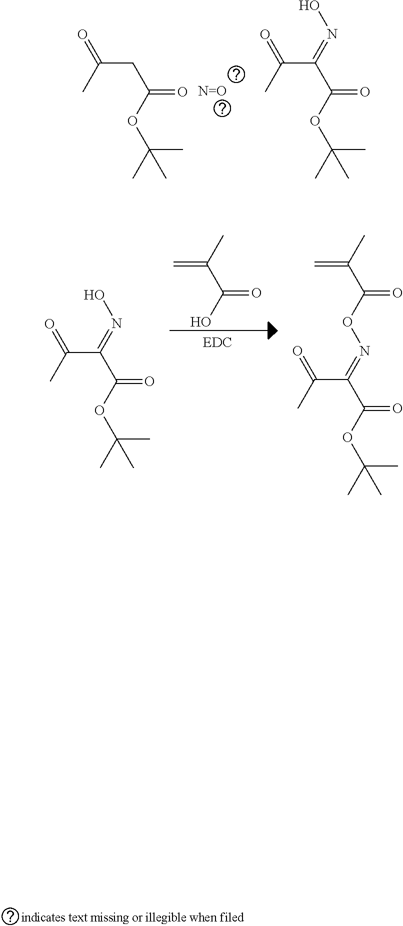

At 0 °C, 15.3 mL of t-butyl acetoacetate was dissolved in 100 mL of acetic acid; and then 7.5 g of NaNO₂ (to generate O═N⁺ in-situ) in 25 mL of water was slowly added over the course of 30 minutes. After reacting for another hour at room temperature, the product was isolated by extraction with methyl t-butyl ether from water and washing with aqueous NaHCO₃. 17.2 g of a slightly yellow solid precursor was obtained after concentration under vacuum. At 0 °C, the solid precursor was dissolved in 100 mL of dichloromethane with 11.0 g of methacrylic acid and 30 mg of 4-pyrrolidinopyridine. Then, 19.1 g of N-(3-dimethylaminopropyl)-N′-ethylcarbodiimide hydrochloride (EDC) was slowly added over 1 hour. After reacting at room temperature for another hour with stirring, the product was isolated by extraction with methyl t-butyl ether from water and washing with aqueous HCl and then aqueous NaHCO₃ solutions. A solid product was obtained after concentration (with 200 mg of BHT) under reduced pressure. The solid was redissolved in 40 mL of warm heptane (~60 °C), the solution was filtered through a 10-micron PE filter with a layer of Na₂SO₄ and cooled to −78 °C, and the solid was collected. The recrystallization process was repeated again to yield 10.3 g of slightly yellow crystalline solid M10.

### Synthesis of ethyl 2-((methacryloyloxy)imino)-3-oxo-3-phenylpropanoate (M13)

In a round bottom flask, 25.0 g of ethyl benzoylacetate was dissolved in 60 mL of acetic acid and stirred at 0 °C. Then, 11.6 g of NaNO₂ in 23 mL of water was slowly added over the course of 60 minutes. After reacting for another hour at room temperature, the product was isolated by extraction with methyl t-butyl ether from water and washing with aqueous NaHCO₃ until the aqueous layer became neutral. 25.3 g of a slightly yellowish solid oxime precursor was obtained.

At 0 °C, 9.0 g of the oxime precursor was dissolved in 70 mL of dichloromethane with 5.0 g of methacrylic acid and 50 mg of 4-pyrrolidinopyridine. Then, 9.0 g of EDC was slowly added over 1 hour. After reacting at room temperature for another hour with stirring, the product was isolated by extraction with methyl t-butyl ether from water and washing with aqueous HCl and NaHCO₃. A waxy product was obtained after concentration (with 20 mg of BHT) under reduced pressure. The material was recrystallized from 30 mL of heptane. The solid was collected and dried to yield 5.5 g of a slightly yellow solid as monomer M13.

### Synthesis of dibutyl 2-((methacryloyloxy)imino)malonate (M14)

In a round bottom flask, 25.0 mL of butyl malonate was dissolved in 100 mL of acetic acid and stirred at 0 °C. Then, 9 g of NaNO₂ in 20 mL of water was slowly added over the course of 2 hours. After reacting for another hour at room temperature, the product was isolated by extraction with methyl t-butyl ether from water and washing with aqueous NaHCO₃ until the aqueous layer became neutral. The mixture was then redissolved in 100 mL of acetic acid and stirred at 0 °C. Then, 18 g of NaNO₂ in 50 mL of water was slowly added over the course of 3 hours. After reacting for another hour at room temperature, the product was isolated by extraction with methyl t-butyl ether from water and washing with aqueous NaHCO₃ until the aqueous layer became neutral. The organic layer was concentrated and found to be a mixture of 60 mol % oxime and 40 mol % starting material. The mixture was used directly without further purification.

At 0 °C, the mixture from the last step was dissolved in 150 mL of dichloromethane with 7.9 g of methacrylic acid and 130 mg of 4-pyrrolidinopyridine. Then, 13.7 g of EDC was slowly added over 45 minutes. After reacting at room temperature for another hour with stirring, the product was isolated by extraction with methyl t-butyl ether from water and washing with aqueous HCl and aqueous NaHCO₃ solutions. The organic layer was concentrated under reduced pressure and purified further using column chromatography (heptane:ethyl acetate=10:1, v v) to yield 16.2 g of a slightly yellowish oil (stabilized with 20 mg of BHT) as a mixture of monomer M14 and dibutyl malonate (about 67:33=mol:mol). The mixture was used directly without further purification.

### Synthesis of neopentyl 2-(((2-fluoroacryloyl)oxy)imino)propanoate (M15)

In a round bottom flask, 53 g of neopentyl alcohol was mixed with 61 g of pyruvic acid, 2.1 g of para-toluenesulfonic acid, and 400 mL of toluene. The reaction was conducted by heating at reflux for 6 hours with a Dean-Stark trap to remove water. The mixture was then washed with water and aqueous NaHCO₃ solution to obtain the crude product, which was further purified by distillation.

In a beaker was added 25 g hydroxylamine hydrochloride and 60 mL of water; 13 g of NaOH pellets were added in portions to prevent overheating. The as-formed hydroxylamine was transferred to a solution containing 30.0 g of neopentyl pyruvate and 100 mL of methanol, and then stirred in a cooling bath at room temperature. The solution was further stirred overnight and the product was isolated by extraction with ethyl acetate from a dilute aqueous solution of HCl. After drying, 32.3 g crystalline solid was obtained as the oxime precursor.

At 0 °C, 14.3 g of the oxime precursor was dissolved in 100 mL of dichloromethane with 8.1 g of 2-fluoroacrylic acid and 60 mg of 4-pyrrolidinopyridine. Then, 16.3 g of EDC was slowly added over 60 minutes. After reacting at room temperature for another hour with stirring, the product was isolated by extraction with methyl t-butyl ether from water and washing with aqueous HCl and NaHCO₃ solutions. The organic layer was concentrated and purified further by recrystallization from heptane (stabilized with ~60 mg of BHT, from 60 °C to 0 °C) to yield 18.5 g of a white crystalline solid as monomer M15.

### Synthesis of 2,8-dimethylnonane-4,5,6-trione-5-(O-methacryoyloxime) (M16)

Into a solution of 24.9 g of 2,8-dimethylnonane-4,6-dione in 80 mL of acetic acid cooled in an ice bath was added a solution of 9.32 g of sodium nitrite in 20 mL of water over 3 minutes. After stirring for 20 hours under nitrogen, 100 mL of water, 100 mL of a brine solution, and 100 mL of a saturated NaHCO₃ solution were added, and the contents were extracted with ethyl acetate. The organic extract was dried with sodium sulfate, filtered, and concentrated under reduced pressure to give 33.2 g of crude material. The crude material was purified first on a silica gel column (hexanes:ethyl acetate=9:1, v:v), and then on a silica gel column eluting with dichloromethane. After concentrating under reduced pressure, 16.7 g of a golden oil was obtained as the oxime precursor.

To a solution of 16.7 g of the oxime precursor in 250 mL of dichloromethane cooled in an ice bath was added 11.0 mL of triethylamine, then 7.7 mL of methacryloyl chloride in 70 mL of dichloromethane were added over 27 minutes under nitrogen. After 2 hours, the reaction was concentrated under reduced pressure. Ethyl acetate (250 mL) was added to dissolve the crude product. The triethylamine hydrochloride salt was filtered and the filtrate was concentrated under reduced pressure to provide 22.2 g of the crude product as a yellow liquid. The crude product was purified on a silica gel column, eluting with hexanes:dichloromethane (1:1=v:v) to afford 16.15 g of monomer M16 as a colorless liquid (stabilized with 35.8 mg of MEHQ). The liquid slowly formed needle-like crystals.

### Synthesis of S-tert-butyl 2-(O-methacryloylimino)-3-oxobutanethioate (M17)

A solution of 23.73 g of 2,2,6-trimethyl-1,3-dioxin-4-one and 15.02 g of tert-butylmercaptan was heated at reflux in 200 mL of toluene under nitrogen. After 20 hours, the reaction was concentrated under reduced pressure. The crude material was loaded onto a silica gel column eluting with 2.5 L of hexanes and then 2 L of hexanes:ethyl acetate (95:5=v:v). The isolated fraction was purified again on a silica gel column. To further purify the compound, 3.10 g of activated carbon was added and stirred at room temperature for 15 minutes. After filtering the solid and concentrating the filtrate under reduced pressure, 16.9 g of a colorless oil was obtained.

To a solution of 16.77 g of the thioester in 50 mL of glacial acetic acid cooled in an ice bath was added a solution of 9.96 g of sodium nitrite in 25 mL of water dropwise over 4 minutes. After stirring for 18 hours, water and brine were added into the reaction, and the mixture was extracted with ethyl acetate. The organic extract was dried with sodium sulfate, filtered, and concentrated under reduced pressure to yield the crude product as an orange liquid. The crude product was purified on a silica gel column eluting with hexanes:ethyl acetate (5:1=v:v). The product was used directly for the next step with a slight impurity. The yield of the oxime precursor was 13.21 g.

To a solution of 13.20 g of the oxime precursor in 315 mL of dichloromethane cooled in an ice bath, was added 9.3 mL of triethylamine and then 6.5 mL of methacryloyl chloride in 15 mL of dichloromethane over 8 minutes. After 1 hour, the reaction was concentrated, and ethyl acetate was added. The insoluble materials were filtered and the filtrate was concentrated under reduced pressure, and the crude product (31.5 mL of a dark amber liquid) was purified using a silica gel column eluting with dichloromethane. After drying, 1.50 g of pure product (stabilized with 9.9 mg of MEHQ) was obtained as a colorless solid. The impure fraction obtained from the column was combined and repurified using a second silica gel column eluting with dichloromethane to afford an additional 3.03 g of colorless solid as the product (stabilized with 12.9 mg of MEHQ).

### Synthesis of methyl 2-((methacryloyloxy)imino)-4,4-dimethyl-3-oxopentanoate (M18)

To a solution of 20.0 g of methyl 4,4-dimethyl-3-oxovalerate in 60 mL of glacial acetic acid in an ice bath was added a solution of 13.08 g of sodium nitrite in 30 mL of water dropwise over 15 minutes. After 2 hours, water and brine were added to the reaction, and the reaction was extracted with ethyl acetate. The organic extract was dried with sodium sulfate, filtered, and concentrated under reduced pressure to provide a cloudy, orange liquid. The crude material was purified on a silica gel column eluting with 5:1 and then 4:1 hexanes:ethyl acetate (v:v). The remaining impure product was repurified on a silica gel column. Combining the fractions yielded 8.3 g of a white solid as the oxime precursor.

To a solution of 8.3 g of the oxime precursor in 100 mL of dichloromethane cooled in an ice bath was added 6.8 mL of triethylamine and then 4.76 mL of methacryloyl chloride in 25 mL of dichloromethane over 15 minutes. After 1.25 hours, the reaction was filtered, and the filtrate was concentrated under reduced pressure. Ethyl acetate was added, and the insoluble materials were filtered. The filtrate was concentrated to give the crude product as a medium-amber liquid. The crude product was purified on a silica gel column eluting with 5:1 hexanes:ethyl acetate (v:v). After drying, 7.5 g of a colorless liquid (stabilized with 7.5 mg of MEHQ) was obtained as the product M18.

### Synthesis of 5-methyl-2,3-hexanedione-2-(methacryloyl)oxime (M19)

To a solution 17.84 g of 5-methyl-2,3-hexanedione in 180 mL of ethanol at room temperature was added a solution of 9.67 g of hydroxylamine hydrochloride in 30 mL of water. Then, a solution of 11.42 g of sodium acetate in 25 mL of water was added thereto. Another 20 mL of water was added, and the reaction was heated at an external temperature of 60 °C for 1 hour and 20 minutes under nitrogen, whereupon the reaction was cooled to room temperature and then concentrated under reduced pressure.

Then, 280 mL of a brine solution was added, and the contents were extracted with ethyl acetate. The combined extracts were dried with sodium sulfate, and concentrated to give an off-white solid. The crude product was purified via silica gel chromatography (6:1 hexanes:ethyl acetate) to give 9.1 g (46%) of the oxime precursor.

To a solution of 11.3 g of the oxime precursor in 150 mL of dichloromethane cooled in an ice bath was added 11.0 mL of triethylamine, and then 7.72 mL of methacryloyl chloride in 50 mL of dichloromethane was added dropwise over 16 minutes. After 1 hour and 50 minutes of stirring, the reaction was filtered, and the filtrate was concentrated under reduced pressure. Ethyl acetate was added to the residue and the solid was filtered. The filtrate was concentrated to give 17.5 g of an amber liquid. The crude product was purified via silica gel chromatography (6:1 hexanes:ethyl acetate) to give 13.2 g (79%) of the product M19 as a colorless liquid (stabilized with 8.5 mg of MEHQ).

### EBL Polymer Synthesis

The EBL polymer examples were synthesized using the general protocol below. The CTA (chain transfer agent, 2-cyanopropyl dodecyl trithiocarbonate), initiator (AIBN is azobisisobutyronitrile, V601 is dimethyl 2,2′-azobis(2-methylpropionate)), monomer 1, optional monomer 2, and optional monomer 3 were dissolved in a solvent. The specific amounts are summarized in Table 1, where the amounts of components are in milligrams and the amount of solvent is in grams. The mixture was degassed by sparging with nitrogen gas for 10-20 minutes. The reaction was then heated to 70-72 °C for 20 hours. Some of the samples were isolated by precipitation into a solvent; the others were used directly as the solution. The polymer molecular weight information (Mₙ and M_{w}/Mₙ) is summarized in Table 2 as determined by gel permeation chromatography (GPC) using polystyrene standards and is reported in Daltons (Da).

TABLE 1

| Poly- | Mono- | Mono- | Mono- | | | |
|---|---|---|---|---|---|---|
| mer | mer 1 | mer 2 | mer 3 | CTA | Initiator | Solvent |
| P1 | M3 | M12 | | [114] | AIBN | PGMEA |
| | [1609] | [2000] | | | [11] | [6] |
| P2 | M3 | M12 | | [126] | AIBN | PGMEA |
| | [2252] | [1200] | | | [12] | [6] |
| P3 | M3 | M11 | | [92] | AIBN | PGMEA |
| | [1284] | [1200] | | | [9] | [5.5] |
| P4 | M2 | M12 | | [126] | AIBN | PGMEA |
| | [1072] | [2000] | | | [12] | [6] |
| P5 | M3 | M6 | M11 | [100] | V601 | Dioxane |
| | [743] | [700] | [522] | | [13] | [2.09] |
| P6 | M3 | M6 | M11 | [99] | V601 | Dioxane |
| | [354] | [869] | [643] | | [14] | [1.99] |
| P7 | M7 | M11 | | [130] | V601 | Dioxane |
| | [760] | [907] | | | [18] | [1.61] |
| P8 | M7 | M11 | | [137] | V601 | Dioxane |
| | [567] | [1011] | | | [33] | [1.62] |
| P9 | M10 | M11 | | [123] | V601 | PGMEA |
| | [1250] | [911] | | | [26] | [2.30] |
| P10 | M10 | M11 | | [126] | V601 | PGMEA |
| | [1510] | [733] | | | [30] | [2.41] |
| P11 | M9 | M11 | | [131] | V601 | PGMEA |
| | [1508] | [478] | | | [22] | [2.29] |
| P12 | M9 | M11 | | [123] | V601 | PGMEA |
| | [1265] | [615] | | | [26] | [2.02] |
| P13 | M4 | | | [84] | V601 | PGMEA |
| | [2330] | | | | [22] | [2.44] |
| P14 | M4 | M11 | | [106] | V601 | PGMEA |
| | [1845] | [236] | | | [28] | [2.10] |
| P15 | M8 | | | [87] | V601 | PGMEA |
| | [1510] | | | | [34] | [3.25] |
| P16 | M8 | M11 | | [88] | V601 | PGMEA |
| | [1240] | [180] | | | [27] | [2.82] |
| P17 | M10 | | | [101] | AIBN | PGMEA |
| | [2850] | | | | [10] | [6.91] |
| P18 | M10 | M11 | | [109] | AIBN | PGMEA |
| | [2747] | [350] | | | [10]) | [7.51] |
| P19 | M10 | M11 | | [107] | AIBN | PGMEA |
| | [2262] | [700] | | | [10] | [7.18] |
| P20 | M5 | | | [90] | V601 | PGMEA |
| | [1790] | | | | [30] | [3.75] |
| P21 | M5 | M11 | | [98] | V601 | PGMEA |
| | [1540] | [232] | | | [41] | [3.79] |
| P22 | M17 | | | [102] | V601 | PGMEA |
| | [1425] | | | | [31] | [1.52] |
| P23 | M17 | M11 | | [126] | V601 | PGMEA |
| | [1500] | [439] | | | [37] | [2.06] |
| P24 | M16 | | | [96] | V601 | PGMEA |
| | [1600] | | | | [24] | [1.53] |
| P25 | M16 | M11 | | [107] | V601 | PGMEA |
| | [1672] | [469] | | | [30] | [2.57] |
| P26 | M14 | | | [110] | V601 | PGMEA |
| | [3000] | | | | [17] | [4.69] |
| P27 | M14 | M11 | | [120] | V601 | PGMEA |
| | [2720] | [400] | | | [12] | [4.88] |
| P28 | M15 | M11 | | [135] | V601 | PGMEA |
| | [2268] | [430] | | | [35] | [2.83] |
| P29 | M18 | M11 | | [93] | V601 | PGMEA |
| | [1129] | [349] | | | [21] | [1.56] |
| P30 | M10 | M11 | M13 | [87] | V601 | PGMEA |
| | [402] | [435] | [1132] | | [30] | [2.06] |
| P31 | M19 | M11 | | [156] | V601 | PGMEA |
| | [2848] | [442] | | | [46] | [3.48] |

TABLE 2

| Polymer | Mₙ (Da) | M_{w}/Mₙ |
|---|---|---|
| P1 | 7031 | 1.24 |
| P2 | 7984 | 1.26 |
| P3 | 8933 | 1.30 |
| P4 | 6848 | 1.30 |
| P5 | 7894 | 1.38 |
| P6 | 7019 | 1.32 |
| P7 | 5398 | 2.29 |
| P8 | 5026 | 1.74 |
| P9 | 4824 | 1.46 |
| P10 | 5458 | 1.52 |
| P11 | 5457 | 1.66 |
| P12 | 5238 | 1.63 |
| P13 | 7227 | 1.31 |
| P14 | 5123 | 1.35 |
| P15 | 4063 | 1.40 |
| P16 | 3847 | 1.37 |
| P17 | 6932 | 1.87 |
| P18 | 6627 | 1.60 |
| P19 | 6358 | 1.54 |
| P20 | 7467 | 1.41 |
| P21 | 6048 | 1.40 |
| P22 | 6063 | 1.58 |
| P23 | 4431 | 1.50 |
| P24 | 7774 | 1.81 |
| P25 | 5959 | 1.48 |
| P26 | 7092 | 1.56 |
| P27 | 6878 | 1.41 |
| P28 | 5398 | 2.29 |
| P29 | 4317 | 1.46 |
| P30 | 6247 | 1.48 |
| P31 | 8845 | 1.82 |

### Comparative Polymer Synthesis (CP1)

A monomer solution was prepared by combining 192.00 g of PGMEA, 133.2 g of (methacryloyloxy)methylene bis(2,2-difluoro-3,3-dimethylbutanoate), and 8.51 g of ethylcyclopentyl methacrylate in a flask and agitating the resulting mixture to dissolve the components. Separately, an initiator solution was prepared by combining 10.72 g of PGMEA and 6.2 g of V601 initiator in a flask. 20.05 g of PGMEA was introduced into a separate reaction vessel and the vessel was purged with nitrogen for 30 minutes. The reaction vessel was next heated to 95 °C with agitation. The monomer solution and initiator solution were then introduced as separate feeds into the reaction vessel over a period of 2.5 hours. Upon completion of the 2.5 hours, the reaction vessel was maintained at 95 °C for an additional 3 hours with agitation, and was then allowed to cool to room temperature. The product was obtained with a M_{w}/Mₙ (kDa) of 9.7/6.2 as determined by GPC using polystyrene standards.

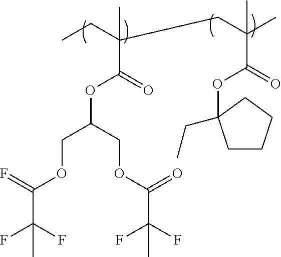

### Preparation of Second Polymer (P32)

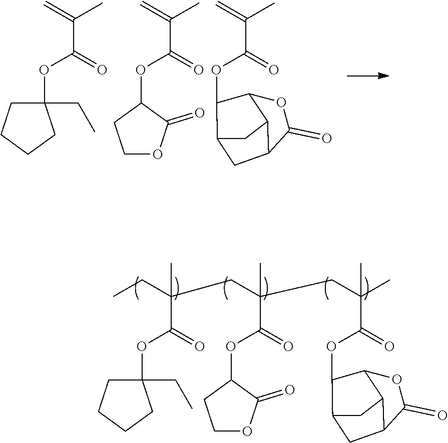

The second polymer (ECPMA/aGBLMA/NLMA at a 40/40/20 molar ratio) was synthesized using the following protocol. The reactor was charged with 11.200 g of PGMEA, which was heated to 80 °C. A monomer feed mixture was prepared as a mixture of 10.245 g of ECPMA, 9.556 g of GBLMA, and 6.244 g of NLMA, and degassed before use. An initiator feed mixture was prepared with 1.541 g of V601 initiator and 13.902 g of PGMEA, and degassed before use. Feeding of the monomer solution and the initiator solution were started at the same time. 5 hours after the feeding started, the reactor was cooled at 1 °C/min to ambient temperature. The polymer was isolated by precipitation into 10 times volume of methanol and dried before use. Molecular weight was determined as M_{w}=8.5 kDa and PDI=1.54 by GPC using polystyrene standards.

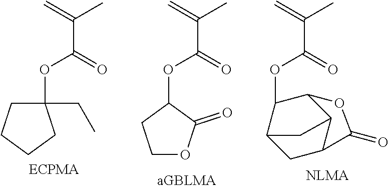

### Coating Compositions

Positive tone photoresist compositions were prepared using the materials and in the amounts indicated in Table 3, where the amounts are expressed in wt % based on 100 wt % of total weight of the solids. The total solids content for the photoresist compositions was 3.10 wt %. The solvent system contained propylene glycol methyl ether acetate (S1) (40 wt %) and 2-hydroxyisobutyric acid methyl ester (S2) (60 wt %). Each mixture was shaken in a 20 mL glass container on a mechanical shaker and filtered through a PTFE disk-shaped filter having a pore size of 0.20 micrometers.

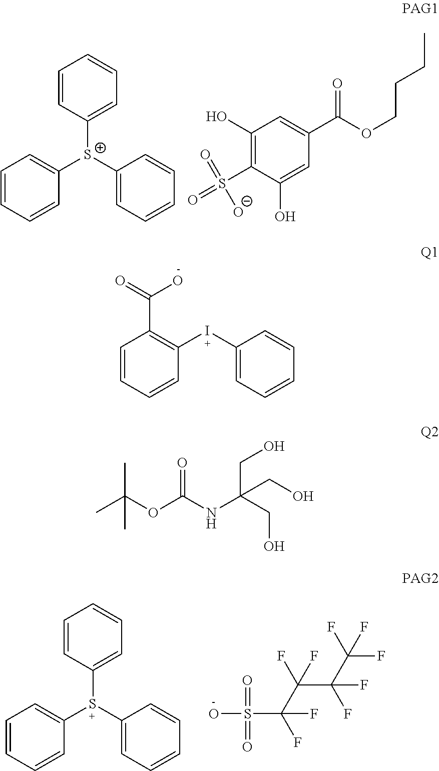 [SMILES: c1ccc([S+](c2ccccc2)c2ccccc2)cc1 ; CCCCOC(=O)c1cc(O)c(S(=O)(=O)[O-])c(O)c1 ; O=C([O-])c1ccccc1[I+]c1ccccc1 ; CC(C)(C)OC(=O)NC(CO)(CO)CO ; c1ccc([S+](c2ccccc2)c2ccccc2)cc1 ; O=S(=O)([O-])C(F)(F)C(F)(F)C(F)(F)C(F)(F)F]

TABLE 3

| Photo- | | | | | | |
|---|---|---|---|---|---|---|
| resist | | | | | | |
| Composi- | First | Second | | | | |
| tion | Polymer | Polymer | PAG | Q1 | Q2 | Solvent |
| CPR1* | CP1 | P32 | PAG2 | [1.56] | [0.97] | S1/S2 |
| | [3.00] | [85.38] | [9.09] | | | [40:60 w/w] |
| CPR2* | CP1 | P32 | PAG1 | [1.56] | [0.97] | S1/S2 |
| | [3.00] | [85.53] | [8.93] | | | [40:60 w/w] |
| PR1 | P1 | P32 | PAG1 | [1.56] | [0.97] | S1/S2 |
| | [3.02] | [85.52] | [8.93] | | | [40:60 w/w] |
| PR2 | P2 | P32 | PAG1 | [1.56] | [0.97] | S1/S2 |
| | [3.03] | [85.51] | [8.93] | | | [40:60 w/w] |
| PR3 | P3 | P32 | PAG1 | [1.56] | [0.97] | S1/S2 |
| | [3.03] | [85.51] | [8.93] | | | [40:60 w/w] |
| PR4 | P4 | P32 | PAG1 | [1.56] | [0.97] | S1/S2 |
| | [3.00] | [85.53] | [8.93] | | | [40:60 w/w] |
| PR5 | P5 | P32 | PAG1 | [1.56] | [0.97] | S1/S2 |
| | [3.00] | [85.53] | [8.93] | | | [40:60 w/w] |
| PR6 | P6 | P32 | PAG1 | [1.56] | [0.97] | S1/S2 |
| | [2.98] | [85.55] | [8.93] | | | [40:60 w/w] |
| PR7 | P7 | P32 | PAG1 | [1.56] | [0.97] | S1/S2 |
| | [3.02] | [85.52] | [8.93] | | | [40:60 w/w] |
| PR8 | P8 | P32 | PAG1 | [1.56] | [0.97] | S1/S2 |
| | [3.00] | [85.53] | [8.93] | | | [40:60 w/w] |
| PR9 | P9 | P32 | PAG1 | [1.56] | [0.97] | S1/S2 |
| | [3.00] | [85.53] | [8.93] | | | [40:60 w/w] |
| PR10 | P10 | P32 | PAG1 | [1.56] | [0.97] | S1/S2 |
| | [3.00] | [85.53] | [8.93] | | | [40:60 w/w] |
| PR11 | P11 | P32 | PAG1 | [1.56] | [0.97] | S1/S2 |
| | [2.98] | [85.55] | [8.93] | | | [40:60 w/w] |
| PR12 | P12 | P32 | PAG1 | [1.56] | [0.97] | S1/S2 |
| | [3.00] | [85.53] | [8.93] | | | [40:60 w/w] |
| PR13 | P13 | P32 | PAG1 | [1.56] | [0.97] | S1/S2 |
| | [3.03] | [85.51] | [8.93] | | | [40:60 w/w] |
| PR14 | P14 | P32 | PAG1 | [1.56] | [0.97] | S1/S2 |
| | [3.00] | [85.53] | [8.93] | | | [40:60 w/w] |
| PR15 | P15 | P32 | PAG1 | [1.56] | [0.97] | S1/S2 |
| | [3.00] | [85.53] | [8.93] | | | [40:60 w/w] |
| PR16 | P16 | P32 | PAG1 | [1.56] | [0.97] | S1/S2 |
| | [3.00] | [85.53] | [8.93] | | | [40:60 w/w] |
| PR17 | P17 | P32 | PAG1 | [1.56] | [0.97] | S1/S2 |
| | [3.00] | [85.53] | [8.93] | | | [40:60 w/w] |
| PR18 | P18 | P32 | PAG1 | [1.56] | [0.97] | S1/S2 |
| | [3.00] | [85.53] | [8.93] | | | [40:60 w/w] |
| PR19 | P19 | P32 | PAG1 | [1.56] | [0.97] | S1/S2 |
| | [3.00] | [85.53] | [8.93] | | | [40:60 w/w] |
| PR20 | P20 | P32 | PAG1 | [1.56] | [0.97] | S1/S2 |
| | [3.00] | [85.53] | [8.93] | | | [40:60 w/w] |
| PR21 | P21 | P32 | PAG1 | [1.56] | [0.97] | S1/S2 |
| | [3.00] | [85.53] | [8.93] | | | [40:60 w/w] |
| PR22 | P22 | P32 | PAG1 | [1.56] | [0.97] | S1/S2 |
| | [3.00] | [85.53] | [8.93] | | | [40:60 w/w] |
| PR23 | P23 | P32 | PAG1 | [1.56] | [0.97] | S1/S2 |
| | [3.00] | [85.53] | [8.93] | | | [40:60 w/w] |
| PR24 | P24 | P32 | PAG1 | [1.56] | [0.97] | S1/S2 |
| | [3.00] | [85.53] | [8.93] | | | [40:60 w/w] |
| PR25 | P25 | P32 | PAG1 | [1.56] | [0.97] | S1/S2 |
| | [3.00] | [85.53] | [8.93] | | | [40:60 w/w] |
| PR26 | P26 | P32 | PAG1 | [1.56] | [0.97] | S1/S2 |
| | [3.00] | [85.53] | [8.93] | | | [40:60 w/w] |
| PR27 | P27 | P32 | PAG1 | [1.56] | [0.97] | S1/S2 |
| | [3.00] | [85.53] | [8.93] | | | [40:60 w/w] |
| PR28 | P28 | P32 | PAG1 | [1.56] | [0.97] | S1/S2 |
| | [3.00] | [85.53] | [8.93] | | | [40:60 w/w] |
| PR29 | P29 | P32 | PAG1 | [1.56] | [0.97] | S1/S2 |
| | [3.00] | [85.53] | [8.93] | | | [40:60 w/w] |
| PR30 | P30 | P32 | PAG1 | [1.56] | [0.97] | S1/S2 |
| | [3.00] | [85.53] | [8.93] | | | [40:60 w/w] |
| PR31 | P31 | P32 | PAG1 | [1.56] | [0.97] | S1/S2 |
| | [3.00] | [85.53] | [8.93] | | | [40:60 w/w] |

*indicates comparative photoresist composition.

### Contact Angle Evaluation

On a TEL ACT-8 wafer track, 8-inch silicon wafers were primed with hexamethyldisilazane (HMDS) at 120 °C for 30 seconds, coated with a photoresist composition listed in Table 3 and soft baked at 90 °C for 60 seconds to give a film layer having a thickness of about 80 nm. The contact angle for each of the polymer solutions was measured on a Kruss contact angle goniometer using deionized Millipore filtered water. Receding angle as coated with soft bake was then measured at the beginning of lateral drop motion before rapid acceleration. Receding angle after develop/DI water rinse was measured with wafers treated with MF™ CD26 TMAH developer (Qnity Electronics, Inc.) for 60 seconds, rinsed with DI water, and dried. For each formulation, two wafers were coated, one without TMAH develop and the other was developed by TMAH with 60 second single puddle. The contact angle as measured before and after development is shown in Table 4.

TABLE 4

| Photoresist | Receding Angle as coated | Receding Angle after |
|---|---|---|
| Composition | with soft bake (°) | develop/DI water rinse (°) |
| CPR1* | 65.30 | 18.1 |
| CPR2* | 68.10 | 17.4 |
| PR2 | 72.27 | 39.7 |
| PR3 | 68.50 | 31 |
| PR4 | 67.10 | 41.4 |
| PR6 | 60.09 | 32.43 |
| PR8 | 62.67 | 41.5 |
| PR9 | 74.63 | 32.16 |
| PR10 | 74.68 | 31.76 |
| PR11 | 77.94 | 30.95 |
| PR12 | 77.76 | 32.81 |
| PR13 | 66.85 | 17.5 |
| PR14 | 65.80 | 22.5 |
| PR15 | 63.25 | 20.6 |
| PR16 | 62.40 | 18.5 |
| PR17 | 67.57 | 17.4 |
| PR18 | 69.66 | 18.1 |
| PR19 | 71.31 | 17.7 |
| PR20 | 67.90 | 17.1 |
| PR21 | 67.60 | 17.3 |
| PR22 | 65.9 | 17.1 |
| PR23 | 68.6 | 19.1 |
| PR24 | 80.8 | 17.5 |
| PR25 | 83.1 | 22.8 |
| PR26 | 72.6 | 17.1 |
| PR27 | 73.6 | 17.8 |
| PR28 | 66.3 | 24.3 |
| PR29 | 61.2 | 36.5 |
| PR30 | 61.7 | 27.7 |
| PR31 | 76.5 | 19.7 |

*indicates comparative photoresist composition.

As seen in Table 4, the exemplary photoresist compositions in a coated dried state exhibited a receding contact angle of greater than 60°.

### Photoresist Formulations and Evaluation

### Photoresist Polymer Preparation

Polymer P33 was synthesized using free radical polymerization as follows. A one-neck 100 mL reactor flask was charged with PGMEA (10.5 g) and purged under nitrogen for 30 minutes with stirring. The reactor flask was heated to 80 °C, followed by charging of monomer M10 (18.33 g, 71.8 mmol), monomer M11 (5.67 g, 30.7 mmol), and PGMEA (40% wt solution) over the course of 4 hours. At the same time, V601 initiator (0.49 g, 2.13 mmol) in PGMEA (20 wt % solution) was charged to the reaction mixture over the course of 3.5 hours. The reaction mixture was then allowed to cool to room temperature after 5 hours. The reaction mixture was used directly. Molecular weight was determined as Mw=8.9 kDa, PDI=1.80, by gel permeation chromatography (GPC) using polystyrene standards.

### Photoresist Formulations

Positive tone photoresist compositions were prepared using the materials and in the amounts indicated in Table 5, where the amounts are expressed in wt % based on 100 wt % of total weight of the solids. The total solids content for the photoresist compositions was 3.10 wt %. The solvent system contained propylene glycol methyl ether acetate (S1) (40 wt %) and 2-hydroxyisobutyric acid methyl ester (S2) (60 wt %). Each mixture was shaken in a 20 mL glass container on a mechanical shaker and filtered through a PTFE disk-shaped filter having a pore size of 0.20 micrometers.

TABLE 5

| Photo- | | | | | | |
|---|---|---|---|---|---|---|
| resist | | | | | | |
| Composi- | First | Second | | | | |
| tion | Polymer | Polymer | PAG | Q1 | Q2 | Solvents |
| CPR3* | CP1 | P32 | PAG2 | [1.56] | [0.97] | S1/S2 |
| | [4.0] | [84.38] | [9.09] | | | [40:60 w/w] |
| PR32 | P33 | P32 | PAG1 | [1.56] | [0.97] | S1/S2 |
| | [4.5] | [84.33] | [8.93] | | | [40:60 w/w] |
| PR33 | P33 | P32 | PAG1 | [1.56] | [0.97] | S1/S2 |
| | [6.0] | [82.92] | [8.93] | | | [40:60 w/w] |

*indicates comparative photoresist composition.

### Photoresist Patterning and Processing

The photoresist compositions were evaluated by lithography. A 300 mm silicon wafer was first spin-coated with a bottom anti-reflective coating (BARC) AR™40 (Qnity Electronics, Inc.) and baked at 205 °C for 60 seconds to form an 80 nm film. A silicon-containing antireflective coating (SiARC) material was then spin-coated on over the BARC layer and baked at 240 °C for 60 seconds to form a 30 nm film. The photoresist composition was then spin-coated on top to form a 90 nm film and soft-baked at 90 °C for 60 seconds. The coated wafer was then exposed with ArF excimer laser at 193 nm using ASML 1900i immersion tool (NA 1.35, a 0.988/0.9). Thereafter, the wafer was subject to post-exposure bake at 95 °C for 60 seconds followed by development with 0.26 N TMAH solution and subsequent water rinse.

The photoresist pattern was observed under CD-SEM (CG4000, Hitachi High-Technologies Corp.). The exposure dose that provides a 1:1 line pattern having a size of 45 nm and 38 nm was reported as sensitivity (mJ/cm²). Exposure latitude (%) was the extent to which a light-sensitive material can be overexposed or underexposed and still achieve an acceptable result. The value was defined as the percentage of exposure dose range of ±10% of the critical dimension (CD) to the exposure dose at CD. 36 of the LS pattern having a target size of 45 nm and 38 nm were determined, where 3a is a scale indicating LWR. The results are shown in Table 6 as “LWR (nm)”. “3σ” indicates a triple value (unit: nm) of the standard deviation (a) determined from measurement results obtained by measuring 20 line positions in the longitudinal direction of the line with a CD-SEM.

TABLE 6

| | 45 nm/90 p | | | 38 nm/76 p | | |
|---|---|---|---|---|---|---|
| | | Exposure | | | Exposure | |
| | Sensitivity | Latitude | LWR | Sensitivity | Latitude | LWR |
| | (mJ/cm²) | (%) | (nm) | (mJ/cm²) | (%) | (nm) |
| CPR3* | 17.6 | 17.1 | 3.6 | 19.4 | 13.0 | 3.5 |
| PR32 | 19.3 | 18.1 | 3.6 | 21.6 | 13.0 | 3.6 |
| PR33 | 18.4 | 18.7 | 3.3 | 20.7 | 13.3 | 3.6 |

*indicates comparative photoresist composition.

As shown in Table 6, the inventive photoresist compositions achieved comparable performance to the comparative photoresist composition in terms of sensitivity, exposure latitude, and LWR.

While this disclosure has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims.

1. A photoresist composition, comprising:
a first polymer comprising a first repeating unit comprising a group of Formula (1) or (2);
a second polymer comprising a first repeating unit comprising an acid-labile group;
a photoacid generator; and
a solvent, 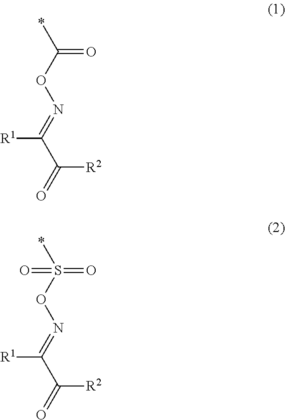
wherein in Formula (1) and Formula (2),
R¹ and R² are each independently hydrogen or an organic group;
R¹ and R² together optionally form a ring;
each of R¹ and R² optionally further comprises one or more divalent linking groups as part of its structure;
one of R¹ or R² is optionally bonded to a backbone of the first polymer; and
* represents a point of bonding in the first repeating unit of the first polymer.

2. The photoresist composition of claim 1, wherein the first repeating unit of the first polymer comprises a group of Formula (1).

3. The photoresist composition of claim 1, wherein the first repeating unit of the first polymer comprises a group of Formula (2).

4. The photoresist composition of claim 1, wherein the first repeating unit of the first polymer is formed from a monomer comprising an ethylenically unsaturated double bond.

5. The photoresist composition of claim 1, wherein a total content of all repeating units comprising a group of Formula (1) or Formula (2) in the first polymer is from 30 to 100 mol % based on total repeating units of the first polymer.

6. The photoresist composition of claim 1, wherein the first polymer is present in the photoresist composition in an amount from 0.1 to 20 wt % based on total solids of the photoresist composition.

7. The photoresist composition of claim 1, wherein the photoresist composition in a coated dried state exhibits a receding contact angle of greater than 60°.

8. The photoresist composition of claim 1, wherein the first polymer is free of —CF₂— and —CF₃ groups that are bonded to a carbon atom.

9. The photoresist composition of claim 8, wherein the first polymer is free of fluorine.

10. The photoresist composition of claim 1, wherein the photoresist composition is free of fluorine.

11. The photoresist composition of claim 1, wherein the first repeating unit of the first polymer comprises a group of Formula (1A) or (2A): 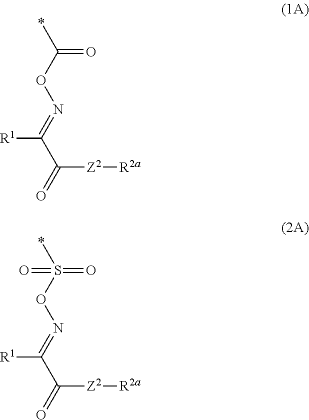
wherein, in Formulae (1A) and (2A),
R^{1a} is hydrogen, substituted or unsubstituted C₁₋₃₀ alkyl, substituted or unsubstituted C₁₋₃₀ heteroalkyl, substituted or unsubstituted C₃₋₃₀ cycloalkyl, substituted or unsubstituted C₂₋₃₀ heterocycloalkyl, substituted or unsubstituted C₂₋₃₀ alkenyl, or substituted or unsubstituted C₂₋₃₀ heteroalkenyl;
R^{2a} is an organic group;
Z² is NR′, O, or S;
R′ is hydrogen, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl;
R^{1a} and R^{2a} together optionally form a ring;
R^{1a} and R^{2a} each optionally further comprises one or more divalent linking groups as part of its structure;
one of R^{1a} or R^{2a} is optionally bonded to a backbone of the first polymer; and
* represents a point of bonding in the first repeating unit of the first polymer.

12. A pattern formation method, comprising:
(a) applying a layer of the photoresist composition of claim 1 on a substrate;
(b) pattern-wise exposing the photoresist composition layer to activating radiation; and
(c) developing the exposed photoresist composition layer to provide a resist relief image.

13. A monomer comprising an ethylenically unsaturated double bond and a group of Formula (1A) or (2A): 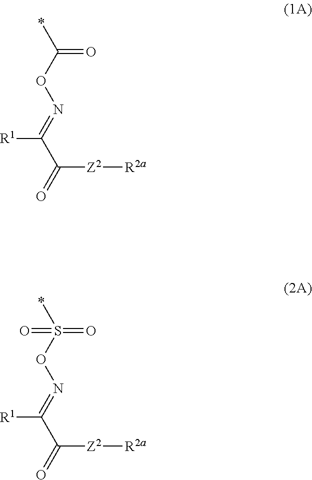
wherein, in Formulae (1A) and (2A),
R¹ is hydrogen, substituted or unsubstituted C₁₋₃₀ alkyl, substituted or unsubstituted C₁₋₃₀ heteroalkyl, substituted or unsubstituted C₃₋₃₀ cycloalkyl, substituted or unsubstituted C₂₋₃₀ heterocycloalkyl, substituted or unsubstituted C₂₋₃₀ alkenyl, or substituted or unsubstituted C₂₋₃₀ heteroalkenyl;
R^{2a} is an organic group;
Z² is NR′, O, or S;
R′ is hydrogen, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl;
R¹ and R^{2a} together optionally form a ring;
R¹ and R^{2a} each optionally further comprises one or more divalent linking groups as part of its structure; and
* represents a point of bonding in the monomer.

14. A polymer comprising a repeating unit comprising a group of Formula (1A) or (2A): 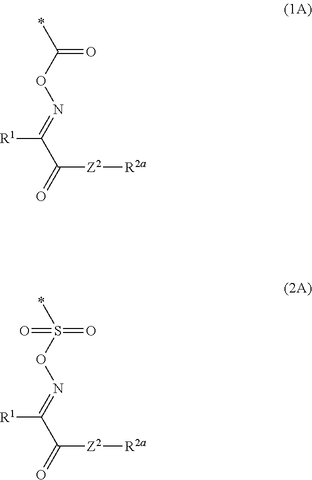
wherein, in Formulae (1A) and (2A),
R¹ is hydrogen, substituted or unsubstituted C₁₋₃₀ alkyl, substituted or unsubstituted C₁₋₃₀ heteroalkyl, substituted or unsubstituted C₃₋₃₀ cycloalkyl, substituted or unsubstituted C₂₋₃₀ heterocycloalkyl, substituted or unsubstituted C₂₋₃₀ alkenyl, or substituted or unsubstituted C₂₋₃₀ heteroalkenyl;
R^{2a} is an organic group;
Z² is NR′, O, or S;
R′ is hydrogen, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl;
R¹ and R^{2a} together optionally form a ring;
R¹ and R^{2a} each optionally further comprises one or more divalent linking groups as part of its structure;
one of R¹ or R^{2a} is optionally bonded to a backbone of the polymer; and
* represents a point of bonding in the repeating unit of the polymer.

15. The method of claim 12, wherein the first repeating unit of the first polymer comprises a group of Formula (1).

16. The method of claim 12, wherein the first repeating unit of the first polymer comprises a group of Formula (2).

17. The method of claim 12, wherein the first repeating unit of the first polymer is formed from a monomer comprising an ethylenically unsaturated double bond.

18. The method of claim 12, wherein a total content of all repeating units comprising a group of Formula (1) or Formula (2) in the first polymer is from 30 to 100 mol % based on total repeating units of the first polymer.

19. The method of claim 12, wherein the photoresist composition in a coated dried state exhibits a receding contact angle of greater than 60°.

20. The method of claim 12, wherein the first polymer is free of —CF₂— and —CF₃ groups that are bonded to a carbon atom.
